SEQUENCE LISTING (11 sequences)

SEQ ID NO: 1 - protein, 200 aa, synthetic construct
  REGION 1..200: note = PhlF repressor protein
MARTPSRSSIGSLRSPHTHKAILTSTIEILKECGYSGLSIESVARRAGASKPTIYRWWTNKAALIAEVYENESEQVRKFPDLGSFKADLDFLLRNLWKVWRETICGEAFRCVIAEAQLDPATLTQLKDQFMERRREMPKKLVENAISNGELPKDTNRELLLDMIFGFCWYRLLTEQLTVEQDIEEFTFLLINGVCPGTQR

SEQ ID NO: 2 - DNA, 600 bp, synthetic construct
  misc_feature 1..600: note = DNA sequence encoding PhlF repressor protein
  source 1..600: mol_type = other DNA
atggcacgtaccccgtcacgtagtagcattggtagcctgcgtagtccgcatacccataaagcaattctgaccagtaccatcgagatcctgaaagaatgtggttatagcggactgagcattgaaagcgttgcacgtcgtgccggagcaagcaaaccgaccatttatcgttggtggacgaataaagcagcactgattgccgaagtgtatgaaaatgaaagcgaacaggtgcgtaaatttccggatctgggtagctttaaagcagatctggattttttactgcgtaatttatggaaagtttggcgtgaaactatttgcggtgaagcatttcgttgtgttattgcagaagctcagctggatcctgcaaccctgacccagttaaaggatcaatttatggaacgtcgtcgtgagatgccgaaaaaactggttgaaaatgccattagcaatggtgaactgccgaaagataccaatcgtgaacttcttctggatatgatttttggtttttgttggtatcgcctgttaaccgaacagctgaccgttgaacaggatattgaagaatttaccttccttctgattaatggtgtttgtccgggtactcagcgt

SEQ ID NO: 3 - DNA, 51 bp, synthetic construct
  misc_feature 1..51: note = PhlF promoter-operator sequence
  source 1..51: mol_type = other DNA
tctgattcgttaccaattgacatgatacgaaacgtaccgtatcgttaaggt

SEQ ID NO: 4 - DNA, 6504 bp, synthetic construct
  misc_feature 1..6504: note = Plasmid containing Cas9 under the control of a PphlF promoter and a constitutive sgRNA guide targeting lacZ
  source 1..6504: mol_type = other DNA
ttttgccgttacgcactactttagtcagttccgcagtaccgtcagtagctgaacaggagggacagtgttgataaagcgccaactttttgcgaaaatgttgcacgtaagacaactttcaccataatgaaataagatcactactattttttgagttatcgagatttcgcaagctaaggaagctaaaatggagaaaaaaatcactggatatactacagttgatataagtcaatggcatcgtaaagaacattttgaggcattccagtcagttgctcagtgcacctataaccaaaccgttcagctggatattacggcctttttaaaaaccgtaaagaaaaataagcacaagttttatccggcctttattcacattcttgcccgcctgatgaatgcacatccggagtttcgtatggcaatgaaagacggtgagctggtgatatgggatagtgttcacccttgttacaccgttttccatgagcaaactgaaacgttttcatcgctctggtcagaataccacgacgatttccggcagtttctacacatatatagtcaagatgtggcgtgttacggtgaaaacttagcatatttccctaaagggtttatcgaaaatatgtttttcgtcagtgccaatccgtgggtgagtttcaccagttttgatttaaacgtagcaaatatggacaacttcttcgcccccgttttcactatgggaaaatattatacgcaaggcgacaaggtgttaatgccactggcgatccaagttcatcatgccgtttgtgatggcttccatgtcggcagaatgcttaatgaattacaacaatactgcgatgagtggcagggcggggcgtaatccggcaaggaaacactgaaaaaagcccgcacctgacagtgcgggctttttttttcgaccaaaggtatgcacatgctatagacttctggtgctacccgactgacagctagctcagtcctaggtataatgctagccgtcgtgactgggaaaacccgttttagagctagaaatagcaagttaaaataaggctagtccgttatcaacttgaaaaagtggcaccgagtcggtgctttttttgagatcaaacatccactagtccctttacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggggcgttcggctaaacccaaaagtaaaaacccgccgaagcgggttttaacgtaaaacaggtgaaactgactttaacgttattagtcaccgcccaactgactcagatcaatacgagtttcatacaggccagtgatgctctggtgaataagagttgcatctaagacttctttagtggaggtgtaacgtttacgatcgatcgtggtgtcgaagtatttgaaggctgccggagcaccaagattagtcagcgtgaacaggtgaatgatgttttctgcctgttcacggatcggtttatcacggtgtttattgtaggcgctcaggactttatccagattcgcgtccgccagaattacgcgtttgctaaactcagaaatctgttcaataatctcgtccagatagtgtttgtgctgttccacgaacagctgtttctgctcattatcttccggactacctttcagtttctcgtagtgcgaggccagatacaggaagttcacgtacttggacggaagggccaattcgttccctttttgcagttcacccgcagacgctaacatacgtttacgaccattttccagttcgaacagggaatacttcggcagtttgataatcagatctttctttacttccttgtaccccttcgcttcgaggaagtcaatcgggtttttttcgaaagagctacgttccataatggtgatgcctaaaagttctttcactgatttcagtttttttgatttacccttttcgactttggcgactaccagaacgctgtatgccacggttggagaatcgaagccaccgtatttcttcggatcccagtctttcttacgcgcgatcagtttgtcggagttgcgtttcggcagaatggattccttgctgaaaccgccagtctgtacctcagtctttttaacgatgtttacttgaggcatggacagaactttgcggacagtagcgaaatcacgacctttgtcccacacgatctcacccgtttcgccgttggtttcgatcagagggcgtttacgaatttcgccattcgctaaggtgatttcggttttgaagaaattcataatgtttgagtagaaaaagtatttagcggttgctttgccaatttcctgttcagatttggcgatcattttgcgcacgtcgtaaactttataatcgccgtacacaaattcgctttccagtttcggatattttttgatcagtgccgtgccaacaactgcattcagatatgcgtcgtgcgcgtggtgatagttgttaatctcgcgcactttatagaattggaaatctttacggaagtcggaaaccagcttagatttcagggtaataaccttaacttcgcggatcagtttgtcattttcatcgtatttagtgttcatacgagagtccagaatctgagcaacatgttttgtaatctggcgagtttctaccagttgacgtttaataaaacccgccttatccagctcagacaaaccgccacgttcagctttggtcaggttatcaaatttacgttgagtaatcagtttcgcgttaagcagttgacgccaatagttcttcattttcttaactacttcttccgatggaacgttgtcactcttcccacggtttttatcgctacgggtgagtactttgttatcaatggaatcatctttcagaaaagactgcggaacaatgtggtcaacgtcgtaatcggagaggcggttgatgtccagctcctgatctacatacatgtcacgaccattttgcaggtagtacaggtacagtttttcattttgcagttgggtattttcaacagggtgttctttcagaatctgagagccaagttctttaataccctcctcgatacgtttcatgcgttcgcgggagtttttctgtcccttctgggtcgtttggttttcacgagccatctcgataacaatgttttccggtttgtgacgacccataactttcacaagctcatccaccactttcacggtttgcaggataccttttttgatcgccggactacctgctaaattggcgatatgctcgtgcagagaatcaccttgaccagaaacctgagctttctggatgtcctctttgaaggtcagggaatcgtcgtgaatcagttgcatgaagttacggtttgcgaagccgtcagatttcaggaagtccagaatagttttaccggactgtttgtcacggataccgttaatcagtttacggctcagacggccccagccagtataacggcgacgtttcagttgtttcattactttatcgtcaaacaggtgagcatacgtcttcaagcgttcttcaatcatttcacgatcttcgaacagagtcagggtcaggacgatgtcctctaagatgtcctcattttcttcgttgtccaagaaatctttgtctttaatgatctttaataaatcgtgatacgtgcctaaacttgcattaaaacgatcttcaactccactaatctcgacggagtcgaagcattcgattttcttgaagtagtcctctttcagctgtttaacggttactttacggttggttttaaacagtaaatcaacaattgcttttttctgttcaccgctcaggaatgcaggtttacgcatgccctcagtcacatactttactttagtcagctcgttgtacacggtgaagtattcgtacagaagggagtgtttcggcagtaccttttcgttcggcaggtttttatcaaagttagtcatgcgctcgatgaaggattgggcgcttgcgcctttgtcaaccacttcctcgaagttccacggggtaatagtttcttcggatttgcgagtcatccacgcgaaacgggagttgccacgcgccagcgggccgacataatacgggatacgaaaggtcaggattttttcgattttttcacgattatctttcaggaacgggtaaaaatcctcttggcgacgcaggatagcgtgcagttcacctaagtggatctggtgcggaatgctaccgttgtcgaaggtacgctgtttacgaagcagatcctcacgattcagtttaacaagtaattcttccgtgccatccattttttctaagattggcttgatgaatttgtagaactcctcttggcttgcacccccgtcaatataacccgcgtagccgtttttggattggtcaaagaagatctccttgtacttctccggcagttgttgccgtaccagcgctttcagaagagtcagatcttggtgatgttcgtcatagcgtttaatcatggatgcagaaagcggtgctttggtgatttcggtattcacacgaagaatatcagacaggagaatagcgtctgataagttctttgccgccagaaacagatcagcgtactgatctccgatctgggccagtaagttatccaagtcatcgtcatacgtatctttgctaagttgaagttttgcgtcctctgccagatcgaagttgcttttgaagttcggggtcagtcccaagctaagagcgatcagattgccgaacagtccgtttttcttctcacccggcagttgcgcaattaagttttccagacgacgggatttggacaggcgtgcggacaagatcgctttcgcgtcaacaccgctagcattaattgggttttcttcaaacagctggttataggtttgcaccagctggatgaacagtttgtctacatcgctgttgtctggattcaggtcgccttcgatcaggaaatgaccacggaacttaatcatatgcgctaaggccagatagatcagacgcagatcggccttatccgtcgaatcgaccagctttttgcgcaggtgatagatagtcgggtatttttcgtgatacgctacttcgtccacaatgttgccgaagattggatgacgttcgtgctttttatcttcttccaccagaaatgattcttccagacggtgaaaaaagctgtcatctactttagccatttcgttagagaagatttcttgcaggtagcagatgcggtttttacgacgggtgtaacggcgacgagcggtgcgtttcagacgtgttgcttctgcggtttcgccggaatcgaacagaagcgcaccgattaagttctttttgatactatggcgatctgtattacccagtaccttgaactttttagacggaactttatattcatcagtgatcaccgcccatccgacgctatttgtgccgatgtctaagcctatgctgtatttcttatccatagaaactttctcctttttaagctccagtaaccttaacgatacggtacgtttcgtatcatgtcaattggtaacgaatcagattcagtacgtcgcgtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcgccaaaacttgtcctttcagtttagccttatccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcaggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcacttacaccctcatcagtgccaacatagtaagccagtatacactccgctagcgctgatgtccggcggtgc

SEQ ID NO: 5 - DNA, 4024 bp, synthetic construct
  misc_feature 1..4024: note = plasmid encoding the PhlF repressor protein
  source 1..4024: mol_type = other DNA
agcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaatcagaattggttaattggttgtggaaacgtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagctgaccaaaacgaaaaaagacgctcgaaagcgtctgttttctggaatttggtagcgattatggcctagttaacgctgagtacccggacaaacaccattaatcagaaggaaggtaaattcttcaatatcctgttcaacggtcagctgttcggttaacaggcgataccaacaaaaaccaaaaatcatatccagaagaagttcacgattggtatctttcggcagttcaccattgctaatggcattttcaaccagttttttcggcatctcacgacgacgttccataaattgatcctttaactgggtcagggttgcaggatccagctgagcttctgcaataacacaacgaaatgcttcaccgcaaatagtttcacgccaaactttccataaattacgcagtaaaaaatccagatctgctttaaagctacccagatccggaaatttacgcacctgttcgctttcattttcatacacttcggcaatcagtgctgctttattcgtccaccaacgataaatggtcggtttgcttgctccggcacgacgtgcaacgctttcaatgctcagtccgctataaccacattctttcaggatctcgatggtactggtcagaattgctttatgggtatgcggactacgcaggctaccaatgctactacgtgacggggtacgtgccatagtacctttctcctctttaatgattcgctagcattgtacctaggactgagctagccgtaaaatcgtgctacatttgaagagataaattgcactgaaatctagaaatattttatctgattaataagatgatcttcttgagatcgttttggtctgcggtcatctgcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaagggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcacccctaccgcatggagataagcatggccacgcagtccagagaaatcggcattcaagccaagaacaagcccggtcactgggtgcaaacggaacgcaaagcgcatgaggcgtgggccgggcttattgcgaggaagcccacggcggcaatgctgctgcatcacctcgtggcgcagatgggccaccagaacgccgtggtggtcagccagaaaacactttccaagctcatcggacgttctttgcggacggtccaatacgcagtcaaggacttggtggccgagcgctggatctccgtcgtgaagctcaacggccccggcaccgtgttggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgttactgcacggcgttcggctgcggcgagcggtatcagctcactcaaaggcggtaatacggttatccacagaatcaggggataacgcaggcctccgaaatcctgcagggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaac

SEQ ID NO: 6 - protein, 213 aa, synthetic construct
  REGION 1..213: note = SrpR repressor protein
MARKTAAEAEETRQRIIDAALEVFVAQGVSDATLDQIARKAGVTRGAVYWHFNGKLEVLQAVLASRQHPLELDFTPDLGIERSWEAVVVAMLDAVHSPQSKQFSEILIYQGLDESGLIHNRMVQASDRFLQYIHQVLRHAVTQGELPINLDLQTSIGVFKGLITGLLYEGLRSKDQQAQIIKVALGSFWALLREPPRFLLCEEAQIKQVKSFE

SEQ ID NO: 7 - DNA, 639 bp, synthetic construct
  misc_feature 1..639: note = DNA sequence encoding SrpR repressor protein
  source 1..639: mol_type = other DNA
atggcacgtaaaaccgcagcagaagcagaagaaacccgtcagcgtattattgatgcagcactggaagtttttgttgcacagggtgttagtgatgcaaccctggatcagattgcacgtaaagccggtgttacccgtggtgcagtttattggcattttaatggtaaactggaagttctgcaggcagttctggcaagccgtcagcatccgctggaactggattttacaccggatctgggtattgaacgtagctgggaagcagttgttgttgcaatgctggatgcagttcatagtccgcagagcaaacagtttagcgaaattctgatttatcagggtctggatgaaagcggtctgattcataatcgtatggttcaggcaagcgatcgttttctgcagtatattcatcaggttctgcgtcatgcagttacccagggtgaactgccgattaatctggatctgcagaccagcattggtgtttttaaaggtctgattaccggtctgctgtatgaaggtctgcgtagcaaagatcagcaggcacagattatcaaagttgcactgggtagcttttgggcactgctgcgtgaaccgcctcgttttctgctgtgtgaagaagcacagattaaacaggtgaaatccttcgaa

SEQ ID NO: 8 - DNA, 67 bp, synthetic construct
  misc_feature 1..67: note = SrpR promoter-operator sequence
  source 1..67: mol_type = other DNA
gattcgttaccaattgacagctagctcagtcctaggtatatacatacatgcttgtttgtttgtaaac

SEQ ID NO: 9 - DNA, 7393 bp, synthetic construct
  misc_feature 1..7393: note = Plasmid p455 containing the Cpf1 nuclease under the control of the PsrpR promoter, a crRNA targeting LacZ and a Lambda phage cos signal
  source 1..7393: mol_type = other DNA
gacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcggcaaaagcaccgccggacatcagcgctagcggagtgtatactggcttactatgttggcactgatgagggtgtaagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacgcgacgtacggtggaagattcgttaccaattgacagctagctcagtcctaggtatatacatacatgcttgtttgtttgtaaactactttcgagtactaacaaagctggtttgaatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagatgccagctggcgtaatagcgaagagtcgagacgaacaataaggcctccctaacggggggccttttttattgataacaaaagtaaagccgaacgccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggactagtggattgcatacctttggtcgaaaaaaaaagcccgcactgtcaggtgcgggcttttttcagtgtttccttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtccctcctgttcagctactgacggtactgcggaactgactaaagtagtgcgtaac

SEQ ID NO: 10 - DNA, 5119 bp, synthetic construct
  misc_feature 1..5119: note = plasmid encoding the SrpR repressor protein
  source 1..5119: mol_type = other DNA
gtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatctagtaggtttcctgtgtgactctagtattattcgaaggatttcacctgtttaatctgtgcttcttcacacagcagaaaacgaggcggttcacgcagcagtgcccaaaagctacccagtgcaactttgataatctgtgcctgctgatctttgctacgcagaccttcatacagcagaccggtaatcagacctttaaaaacaccaatgctggtctgcagatccagattaatcggcagttcaccctgggtaactgcatgacgcagaacctgatgaatatactgcagaaaacgatcgcttgcctgaaccatacgattatgaatcagaccgctttcatccagaccctgataaatcagaatttcgctaaactgtttgctctgcggactatgaactgcatccagcattgcaacaacaactgcttcccagctacgttcaatacccagatccggtgtaaaatccagttccagcggatgctgacggcttgccagaactgcctgcagaacttccagtttaccattaaaatgccaataaactgcaccacgggtaacaccggctttacgtgcaatctgatccagggttgcatcactaacaccctgtgcaacaaaaacttccagtgctgcatcaataatacgctgacgggtttcttctgcttctgctgcggttttacgtgccatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcactcttcctttttcaatattattgaacctgaggtctgtttatcgaattaattgcagatataaaaaaaccaaccgtaagggttggttttttcttgggatttttggtcggcacgagaggatttgaacctccgacccccgacaccccatgacggtgcgctaccaggctgcgctacgtgccgactcgtggctgctaatactaccgttttccacaccgattgcaagtaagatatttcgctaactgatttataattaatcagttagcgataaaacgcttctcgtacaacgctttctggtgaatggtgcgggaggcgagacttgaactcgcacaccttgcggcgccagaacctaaatctggtgcgtctaccaatttcgccactcccgcaaaaaaagatggtggctacgacgggattcgaacctgtgaccccatcattatgagtgatgtgctctaaccaactgagctacgtagccatcttttttttcgcgataccttatcggcgttgcggggcgcattatgcgtatagagccttgcagcgtcaacctctttttcaaggaaaattgctcgaaagtgactgtttggttaggttgcgaacagcgaaccatgacgaactgtaaatctacggaatgcttgatattcaggggatttttgcggactggtacggatgggagcgaactgataaatggtgtcccctgcaggaatcgaacctgcaattagcccttaggaggggctcgttatatccatttaactaagaggacaatgcggcatgagtatacccgctaatggagtgcggggtaagtacgttgccgctcgattgcttaaaccctcgccatttatgccgggtttttataatttttcttaatgttttccgcacgttctgctttttggacgtcatcgattgtccctctaagacacggataaatcggtgatatcaccacatcaaccaggcaacatgcccgacttgttgaatgcaataaacagaaggaaaaaacagggaggagaaaagaagtggtgctgataggcagattcgaactgccgacctcacccttaccaagggtgcgctctaccaactgagctatatcagcacatcttggagcgggcagcgggaatcgaacccgcatcatcagcttggaaggctgaggtaatagccattatacgatgcccgcatcctggaactcggctacctgattttcattctgcactgaatatcgagagaagctctctttattcgagccggtaagcgaacttatcgtctcgggctacgccatcgcgtggccgaaattggtggtgggggaaggattcgaaccttcgaagtctgtgacggcagatttacagtctgctccctttggccgctcgggaaccccaccggacttgatggtgccgactaccggaatcgaactggtgacctactgattacaagtcagttgctctacctactgagccaagtcggcatcaagtagcgcgcactctatggagacatgcgagttcatgcaactaaaaaattgcataatttgttttattggtcacattttatgcgacacgatgaagaaacagcctaggtacctcatgagcccgaagtggcgagcccgatcttccccatcggtgatgtcggcgatataggcgccagcaaccgcacctgtggcgccggtgatgccggccacgatgcgtccggcgtagaggatccgtcacataccaaggcggctaagcgagcagatggaacatcaacgcctgcggtcaggaagatgcgcatcgacagcaagacatcatggctatcagcttgtcgtcgtgcaggaattgaagatttccgtttccatgacctcagacacacctgggcaagctggctgattcagtcaggcgtcccattatcagtgcttcaggaaatgggcggatgggagtccatagaaatggttcgtaggtatgctcaccttgcgcctaatcatttgacagagcatgcgaggaaaatagacgacatttttggtgataatgtcccaaatatgtcccactctgaaattatggaggatataaagaaggcgtaactgattgaattgtaatggcgcgccctgcaggattcgaacctgcggcccacgacttagaaggtcgttgctctatccaactgagctaagggcgcgttgataccgcaatgcggtgtaatcgcgtgaattatacggtcaacccttgctgagtcaatggcttttgatctggttgctgaacaagtgaacgaccgcgtctgattttctgatttatttcgctatagcggcaaacaaacgcacaccgctgcgcgtctgaatcaagaaaacccgtattttcatgtatcaaagtgacctgcagccaagcttggattgcgacacggagttactttataatccaatcgattggccccttagctcagtggttagagcaggcgactcataatcgcttggtcgctgcttcaagtccagcaggggccaccagcggccgcaaaggctgacgagaaatcgtcagcctttttaagctttaagccgaattccagcacactggcggccgttactagtggatccgagctcggtaccaagcttatcgatgataagctgtcaaacatgagaattacaacttatatcgtatggggctgacttcagctgaaacctcaggcatttgagaagcacacggtcacactgcttccggtagtcaataaaccg

SEQ ID NO: 11 - DNA, 12335 bp, synthetic construct
  misc_feature 1..12335: note = Plasmid comprising the PsrpR-Cpf1-LacZ circuit
  source 1..12335: mol_type = other DNA
ctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagatcccgatgtacgcgcgcgtggatgagtctaagaactttaaataatttgtctgtatattatttcgagacgaacaataaggcctccctaacggggggccttttttattgataacaaaagtaacttcgagcttgtctacctcctagcaccattattgcaattaataaacaactaacggacaattctacctaacagttttcatatatgacgagcagttaagtgatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagtt

(19) United States
(12) Patent Application Publication
(10) Pub. No.: US 2024/0218335 A1
(43) Pub. Date: Jul. 4, 2024

(54) TRANSCRIPTIONAL CONTROL IN PROKARYOTIC CELLS USING DNA-BINDING REPRESSORS

(71) Applicant: Eligo Biosciecne, Paris (FR)

(72) Inventors: Jesus Fernandez Rodriguez, Paris (FR); Antonina Krawczyk, Paris (FR); Xavier Duportet, Paris (FR)

(21) Appl. No.: 18/598,135

(22) Filed: Mar. 7, 2024

Related U.S. Application Data
(63) Continuation of application No. 18/154,299, filed on Jan. 13, 2023, now Pat. No. 11,970,716.
Application No. 18/154,299 is a division of application No. 16/819,935, filed on Mar. 16, 2020, now Pat. No. 11,584,918.
(60) Provisional application No. 62/818,903, filed on Mar. 15, 2019.

(51) Int. Cl. C12N 7/00; C12N 9/22; C12N 15/10; C12N 15/87

(52) U.S. Cl. CPC C12N 7/00; C12N 9/22; C12N 15/102; C12N 15/87; C12N 2310/20

(57) ABSTRACT

The present disclosure relates generally to methods and compositions for transferring a genetic circuit from one prokaryotic cell (“donor cell”) to another prokaryotic cell (“recipient cell” or “target cell” which are used interchangeably herein). More specifically, the present disclosure relates to prokaryotic donor cells comprising (i) a genetic circuit of interest and (ii) one or more expressed transcriptional repressor proteins and the use of said donor cells in the efficient transfer of the genetic circuit into a prokaryotic recipient cell. The genetic circuit includes nucleic acid sequences encoding a RNA molecule or protein of interest.

### CROSS-REFERENCE TO RELATED APPLICATION

This application is a continuation of application Ser. No. 18/154,299 filed Jan. 13, 2023, which is a divisional application of Ser. No. 16/819,935 filed Mar. 16, 2020 which claims benefit and priority to U.S. Provisional Application No. 62/818,903, filed Mar. 15, 2019 which is incorporated herein by reference in its entirety.

### REFERENCE TO SEQUENCE LISTING SUBMITTED VIA EFS-WEB

The application contains a Sequence Listing which has been submitted electronically in .XML format and is hereby incorporated by reference in its entirety. Said .XML copy, created on Jan. 12, 2023, is named “2643-5 DIV TK1.xlm”. The sequence listing contained in this .XML file is part of the specification and is hereby incorporated by reference herein in its entirety.

### TECHNICAL FIELD

The present disclosure relates generally to methods and compositions for transferring a genetic circuit from one prokaryotic cell (“donor cell”) to another prokaryotic cell (“recipient cell” or “target cell” which are used interchangeably herein).

### BACKGROUND

Inducible systems that control the transcription from a given promoter are useful tools in molecular biology [1]-[3]. In general, these systems are composed of a protein repressor whose binding to its cognate DNA operator is dependent on the presence of an inducer. In the absence of inducer, the repressor is able to recognize its operator, preventing transcription from a promoter; when the inducer is present, the repressor is no longer able to recognize its operator, either by binding of the inducer to a domain of the repressor or by changing its 3D conformation, hence activating transcription. Inducers can be chemical (IPTG, sugars, small molecules, proteins, etc.) or physical (heat, light, pressure, pH etc.). There are many other ways to prevent transcription or to reduce the amount of protein being made in a cell: conditional degradation of proteins, reduction of plasmid copy numbers in the production strain, etc. These do not repress the promoter per se but act upon other components of the genetic circuit [22].

It may, in some instances, be desirable to control the transcription/translation of a specific gene in a host while being able to activate it in a target bacterium in the absence of inducer. For example, in the case of phagemid transduction of proteins and/or nucleic acids that are toxic to the target bacterium, such as for example CRISPR-Cas9-containing circuits [14], it is imperative that the strain being used to produce the particles does not express the toxic protein and/or nucleic acid encoded by the circuit: otherwise, the production strain would be killed or critically damaged. However, it is necessary that the toxic protein and/or nucleic acids are expressed/transcribed in the target strain to induce either cell death such as sequence-specific cell death or any other type of desired function in the target strain. This would also be true for any other toxic component that will be injected in a target strain, which could also render it toxic for the production strain. Additionally, it has been shown that the constitutive expression of components in genetic circuits, especially if they impose a burden (such as toxic components, or any component having unwanted function in the production strain) is disadvantageous for the cell and they tend to be deleted or mutated, causing undesired breakage of designs and components [15]. In that sense, having a conditional repressor that acts only in the production strain is advantageous from the engineering/manufacturing point of view.

A proposed solution for the specific case of genetic circuit transduction has been offered and involves positive regulators (a phage polymerase and its cognate promoter). The target cells may contain either the promoter or the polymerase only, with the other component being transduced by a phagemid [12][13]. However, this is not a practical approach when working with wild-type strains, because of difficulties to transform one of the components, or in environments where it is not possible to pre-load the target cells with one of the circuit constituents (for example, the gut environment).

Another possible solution for this would be to express the repressor controlling the expression of the toxic component in trans, i.e., not encoded in the circuit to be packaged. In this case, the repressor would only be present in the production strain but not in the target strain. There is, however, a main problem associated with this approach: since strains used to produce phagemids are the same species (or at least very closely related) as those that need to be targeted, the repressor used needs to be carefully chosen. For example, using AraC or LacI would effectively repress the transcription of toxic components in the production strain in trans, and phagemids produced in this way would inject a “naked”, constitutive promoter into the target strains. However, if these are wild-type strains, they will most probably contain their own genes for LacI or AraC, so the injected promoter would be immediately repressed. Even antibiotic-induced systems, such as TetR, are very commonly found in many wild-type strains. The same can happen when using a phage master-repressor-promoter pair: wild-type strains carry a number of prophages in their genomes, and many of these could encode repressors that recognize the promoter being used.

### SUMMARY

Described herein are novel methods and compositions for use in transferring a genetic circuit of interest from a prokaryotic cell (referred to herein as a “donor cell”) to another prokaryotic cell (referred to herein as a “recipient cell” or “target cell”). In certain aspects, the prokaryotic cells are bacterial cells. Further, the genetic circuit comprises a nucleic acid of interest under the transcriptional control of a repressor binding sequence (also referred to herein as promoter/operator). Said nucleic acid sequence of interest may encode a protein or RNA of interest.

The disclosed methods and compositions are based on the expression of one or more repressor proteins in the donor cell, and the presence of a repressor binding sequence positioned within the genetic circuit, that function to repress transcription of the nucleic acid sequence of interest when present in the donor cell. The genetic circuit does not encode said one or more repressor proteins. Thus, upon transfer of the genetic circuit into the recipient cell, said recipient cell being chosen for its lack of expression of said one or more repressor proteins, the nucleic acid sequence of interest is then transcribed.

Methods are provided for the transfer of a genetic circuit from a donor cell to a recipient or target cell. Said transfer may be achieved by a variety of different methods. Such as non limitating examples of transfer include bacterial transduction, conjugation and transformation. In one specific aspect, bacterial delivery vehicles, such as bacteriophage scaffolds, are assembled in the donor cell as a means for efficient transfer of the genetic circuit into a recipient or target cell. In such instances, the donor cell comprises prophage sequences that provide in trans the bacteriophage components, such as capsid proteins, required for assembly of the genetic circuit into bacteriophage particles. Further, the genetic circuit is engineered to contain cis acting packaging signals that mediated the encapsidation of the genetic circuit into the capsid.

Thus, the present disclosure relates to a method of transferring a genetic circuit from a donor cell to a target cell comprising contacting the donor cell with the target cell for a sufficient amount of time to allow transfer of the genetic circuit into the target cell wherein said donor cell expresses a repressor protein, that is not encoded by the genetic circuit and is absent in the target cell and wherein the genetic circuit comprises a nucleic acid sequence of interest under the transcriptional control of a repressor binding sequence. The donor cell may be a bacterial donor cell and the target cell may be a bacterial target cell. The genetic circuit may be packaged within a bacterial delivery vehicle before transfer. In an embodiment, the genetic circuit packaged in the delivery vehicle is a packaged phagemid. The nucleic acid sequence of interest may encode a protein of interest and/or a RNA molecule of interest. In particular, the nucleic acid sequence of interest may encode a protein (i) which is a toxic protein, such as a protein which is toxic to a bacterial cell, in particular to the target cell, more particularly a toxic protein selected from the group consisting of holins, endolysins, restriction enzymes and toxins affecting the survival or the growth of the target cell, (ii) which is a nuclease, such as a CRISPR nuclease, and/or which is a therapeutic protein. Alternatively, or additionally, the nucleic acid sequence of interest may encode a RNA molecule of interest, in particular selected from the group consisting of mRNA, crRNA, tRNA, iRNA, asRNA, ribozyme RNA, guide RNA and RNA aptamers. In a particular embodiment, the nucleic acid sequence of interest encodes a CRISPR nuclease and the genetic circuit further comprises a nucleic acid sequence encoding a guide RNA. In an embodiment, said nucleic acid sequence encoding a guide RNA is under the transcriptional control of a constitutive promoter. In some embodiments, the nucleic acid sequence of interest is a nucleic acid encoding a RNA such as a mRNA, crRNA, tRNA, iRNA (interference RNA), asRNA (anti-sense RNA), ribozyme RNA, RNA aptamer or a guide RNA, a CRISPR locus, a toxin gene, a gene encoding an enzyme such as a nuclease or a kinase, a gene encoding a nuclease selected from the group consisting of a Cas nuclease, a Cas9 nuclease, a TALEN, a ZFN and a meganuclease, a gene encoding a recombinase, a bacterial receptor, a membrane protein, a structural protein or a secreted protein, a gene encoding resistance to an antibiotic or to a drug in general, a gene encoding a toxic protein or a toxic factor, and a gene encoding a virulence protein or a virulence factor, or any of their combinations. More particularly, the nucleic acid sequence of interest may be selected from the group consisting of a nucleic acid encoding one or more of the following: Cas nuclease, Cas9 nuclease, guide RNA, CRISPR locus, toxin, enzyme, nuclease, a kinase, TALEN, ZFN, meganuclease, recombinase, bacterial receptor, membrane protein, a structural protein, secreted protein, protein conferring resistance to an antibiotic or a drug, a toxic protein or a toxic factor, virulence protein, and virulence factor. In an embodiment, the repressor protein is selected from the group consisting of the repressor proteins listed in Table 1. In an embodiment, the repressor proteins are selected from the group consisting of PhlF, SrpR, LitR, PsrA, AmeR, McbR, QacR, TarA, ButR, Orf2 and ScbR.

Methods are also provided for production of bacterial delivery vehicles, packaged phagemids for example, for use in efficient transfer of a desired genetic circuit into a recipient or target cell. The disclosure relates to the use of donor cells as described herein, that express one or more repressor proteins for the controlled expression of a nucleic acid of interest, such as a nucleic acid encoding a toxic or unwanted protein and/or RNA molecule, during production of the bacterial delivery vehicle of interest. The repressed transcription of the nucleic acid of interest in the donor cell may result from the functional positioning of the repressor binding sequence in close proximity to the nucleic acid of interest. Alternatively, the repressed transcription of the nucleic acid of interest in the donor cell may result from the functional positioning of the repressor binding sequence away from the nucleic acid of interest. In contrast, once transferred to the recipient or target cell, the nucleic acid of interest is transcribed due to the absence of said one or more repressor proteins in said cell and the absence of nucleic acid sequence(s) encoding said repressor protein(s) within the genetic circuit.

In one aspect, methods of producing delivery vehicles using the donor cells disclosed herein are provided. In certain embodiments, the delivery vehicles are prepared by introducing the genetic circuit of interest described herein into a donor cell under conditions that permit formation of the delivery vehicles. For example, in certain embodiments, the method comprises (i) introducing into a donor cell the genetic circuit of interest; and (ii) allowing a sufficient amount of time for replication of the genetic circuit of interest and packaging of the genetic circuit into the delivery vehicles. In one aspect, the method may further comprise the step of collecting and, optionally, purifying the delivery vehicles. In particular, the present disclosure relates to a method of producing delivery vehicles comprising i) introducing a genetic circuit into a donor cell expressing a repressor protein, wherein said genetic circuit comprises a nucleic acid of interest under the transcriptional control of a repressor binding sequence recognized by said repressor protein and the repressor protein is not encoded by the genetic circuit; and allowing a sufficient amount of time for replication of the genetic circuit of interest and packaging of the genetic circuit into the delivery vehicles. The method may further comprise a step of collecting the delivery vehicles and optionally a step of purifying the delivery vehicles. In an embodiment, in said method, the donor cell, such as a bacterial donor cell, comprises prophage sequences encoding proteins required in trans for assembly of the genetic circuit into a delivery vehicle. In an embodiment, the genetic circuit is a phagemid and/or the delivery vehicle is a bacterial delivery vehicle, such as a bacteriophage. In an embodiment, the delivery vehicles may be to be used in a target cell, for example, a bacterial target cell, which does not express the repressor protein. The present disclosure also relates to the delivery vehicle obtained by said method. The present disclosure also relates to a genetic circuit comprising a transcriptional promoter controlled by a repressor protein and a nucleic acid sequence of interest placed under the control of said transcriptional promoter, said genetic circuit not encoding the repressor protein and being packaged within a bacterial delivery vehicle. In an embodiment said genetic circuit is a phagemid. It also relates to the use of a delivery vehicle or a genetic circuit as described herein to transfer a genetic circuit from a donor cell to a target cell, wherein the donor cell expresses the repressor protein and the target cell does not express said repressor protein.

In yet another aspect, the disclosure provides donor cells comprising one or more expressed repressor proteins and a genetic circuit of interest. In an embodiment, the genetic circuit of interest comprises a nucleic acid sequence of interest placed under the transcriptional control of a repressor binding sequence recognized by said one or more expressed repressor proteins. Further, in certain embodiments where delivery vehicles are produced, the donor cell may also comprise prophage sequences encoding proteins required in trans for assembly of the genetic circuit of interest into a delivery vehicle. Such proteins include, for example, structural bacteriophage proteins, e.g., capsid proteins. In some particular aspects, the present disclosure relates to a donor cell comprising a genetic circuit comprising a transcriptional promoter controlled by a repressor protein that is expressed by said donor cell but is not encoded by the genetic circuit. In an embodiment, the genetic circuit comprises a nucleic acid sequence of interest placed under the control of the transcriptional promoter. The donor cell may further comprise prophage sequences that provide in trans the bacteriophage components required for assembly of the genetic circuit into bacteriophage particles. The genetic circuit may further comprise cis acting packaging signals that mediate encapsidation of the genetic circuit into a capsid.

The donor cells of the present disclosure comprise a genetic circuit of interest. In certain embodiments, the genetic circuit comprises an expression or transcription cassette having a nucleic acid of interest under the transcriptional control of a repressor binding sequence. Such nucleic acids of interest are selected, for example, from the group consisting of a nucleic acid encoding a RNA such as a mRNA, crRNA, tRNA, iRNA (interference RNA), asRNA (anti-sense RNA), ribozyme RNA, RNA aptamer or a guide RNA, a CRISPR locus, a toxin gene, a gene encoding an enzyme such as a nuclease or a kinase, a gene encoding a nuclease selected from the group consisting of a Cas nuclease, a Cas9 nuclease, a TALEN, a ZFN and a meganuclease, a gene encoding a recombinase, a bacterial receptor, a membrane protein, a structural protein or a secreted protein, a gene encoding resistance to an antibiotic or to a drug in general, a gene encoding a toxic protein or a toxic factor, and a gene encoding a virulence protein or a virulence factor, or any of their combination. In an embodiment, the nucleic acid of interest encodes a therapeutic protein. In another embodiment, the nucleic acid encodes an anti-sense nucleic acid molecule. In some embodiments, the nucleic acid of interest encodes two or more molecules of interest. In particular, one of these molecules may be a nuclease, for instance a Cas nuclease, and another may be a nucleic acid molecule such as a guide RNA. In one aspect, the nucleic acid of interest encodes a nuclease that performs cleavage of a recipient or target cell genome or recipient or target cell plasmid. In some aspects, the cleavage occurs in an antibiotic resistant gene. In another embodiment, the nucleic acid of interest encodes a nuclease that targets cleavage of a recipient or target cell genome and said nuclease is designed to stimulate a homologous recombination event for insertion of a nucleic acid of interest into the genome of the cell.

In an embodiment, in the provided methods or the provided donor cell, the genetic circuit is a phagemid, and/or the donor cell is a bacterial cell, for example, a bacteria cell from the E. coli specie.

The present disclosure also provides pharmaceutical or veterinary compositions comprising donor cells, or one or more of the bacterial delivery vehicles assembled in said donor cells, and a pharmaceutically-acceptable carrier. It is also provided a pharmaceutical or veterinary composition comprising a delivery vehicle and/or a genetic circuit as disclosed herein, and a pharmaceutical acceptable excipient. Also provided is a method for treating a disease or disorder caused by bacteria, for example, a bacterial infection, comprising administering to a subject having said disease or disorder in need of treatment the provided pharmaceutical or veterinary composition. Further provided is (i) a pharmaceutical or veterinary composition as disclosed herein for use as a medicament, and in particular in the treatment of a disease or disorder caused by bacteria, for example, a bacterial infection, and (ii) the use of a pharmaceutical or veterinary composition as disclosed herein for the manufacture of a medicament for treating a disease or disorder caused by bacteria, such as a bacterial infection. A method for reducing the amount of virulent and/or antibiotic resistant bacteria in a bacterial population, in particular in a subject having a bacterial infection, is provided comprising contacting the bacterial population with the disclosed compositions herein. Further provided is the use of a pharmaceutical or veterinary composition as disclosed herein for the manufacture of a medicament for reducing the amount of virulent and/or antibiotic resistant bacteria in a bacterial population, in particular in a subject having a bacterial infection.

The present disclosure further provides kits for use in the transfer of a genetic circuit of interest from a donor cell to a recipient or target cell. In one embodiment, the kit comprises (i) a donor cell expressing a repressor protein; and (ii) a genetic circuit of interest. Said genetic circuit may comprise an expression cassette into which a nucleic acid of interest may be inserted in functional proximity to a repressor binding sequence recognized by said repressor protein.

Optionally, the donor cell of the kit may contain prophage sequences for assembly of delivery vehicles, for example, bacteriophages proteins for packaging of the genetic circuit of interest. The kit may further comprises a recipient or target cell wherein said recipient or target cell fails to express the repressor protein thereby permitting expression of the nucleic acid of interest following transfer into said cells.

### BRIEF DESCRIPTION OF FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1 depicts conditional transcriptional control with an interspecific repressor. On the left, the donor strain, containing a packaging prophage and the PhlF repressor in trans. The genetic circuit carries the packaging signal and a nucleic acid of interest (encoding an actuator protein) under the control of a P_{phlF} promoter. Upon packaging of the genetic circuit, target or recipient cells can be transduced and the P_{phlF} promoter will be active since recipient cells lack the PhlF repressor (not present in E. coli). Note that the PhlF repressor can be replaced with a dCas9+gRNA targeting the promoter, RBS or sequence of the actuator.

FIG. 2 depicts transformation of Cas9-containing genetic circuits. Plasmids containing Cas9 under the control of a P_{phlF} promoter and a constitutive sgRNA guide targeting lacZ were transformed into MG1655 (left panels) or MG1656 (right panels). Empty cells (not carrying any other plasmid) are shown on the top; transformed cells containing an extra plasmid encoding the PhlF repressor are shown at the bottom.

FIG. 3 depicts transduction of Cas9-containing genetic circuits. Phagemids containing Cas9 under the control of a P_{phlF} promoter and a constitutive sgRNA guide targeting lacZ were transduced into MG1655 (left panel), MG1655 with the PhlF repressor encoded in a plasmid (center) or MG1656 (right panel).

FIG. 4 depicts transformation of Cpf1-containing genetic circuits. Plasmids containing Cpf1 under the control of a P_{srpR} promoter and a constitutive crRNA guide targeting lacZ (p455) were transformed into MG1655 (left panels) or MG1656 (right panels). Empty cells (not carrying any other plasmid) are shown on the top row; transformed cells containing an extra plasmid encoding the SrpR repressor (pRARE4-SrpR-1.0) are shown at the bottom.

FIG. 5 depicts packaged phagemids containing Cpf1 under the control of a P_{srpR} promoter and a constitutive crRNA guide targeting lacZ (p455) transduced into MG1655 (left panel), MG1655 with the SrpR repressor encoded in a plasmid (center, pRARE4-SrpR-1.0) or MG1656 (right panel).

FIG. 6 depicts comparison of colony size of strains with or without the SrpR repressor. Both strains were transformed with a P_{srpR}-Cpf1-LacZ genetic circuit (p841). Top panels, cells containing the SrpR in the genome. Bottom panels, cells without the SrpR repressor. Incubation times are shown at the top.

### DETAILED DESCRIPTION

Disclosed herein are novel approaches for delivery of a genetic circuit into a recipient or targeted cell. In one embodiment, novel methods are provided for production of bacterial delivery vehicles for use in efficient transfer of a desired genetic circuit into a target cell. The methods and compositions of the present disclosure are based on the use of a donor cell that expresses one or more repressor proteins for the controlled expression of a protein, such as a toxic protein, and/or a nucleic acid, such as a RNA molecule. This is particularly important, for example, when the genetic circuit is designed to express a toxic protein and/or RNA molecule.

Donor cells expressing one or more repressor proteins are provided. In preferred embodiments, the term “donor cell” refers to donor bacterial cells. As used herein, the term “repressor protein” refers, for example, to a protein that binds to a specific site (herein “repressor binding sequence”) on a nucleic acid and prevents transcription of nearby genes. Typically, a repressor protein is a DNA-binding protein that blocks the attachment of RNA polymerase to the promoter through its binding to a repressor binding sequence (an operator), thus preventing transcription of the genes. Said donor cells may be cells that naturally express one or more repressor proteins. Alternatively, the donor cells may be recombinantly engineered to express one or more repressor proteins. Additionally, the provided donor cells comprise a genetic circuit of interest wherein said genetic circuit contains a nucleic acid of interest under the transcriptional regulation of a repressor binding sequence and does not contain a nucleic acid encoding a repressor protein which is able to bind to said repressor binding sequence. At least one repressor protein expressed by the donor cell is able to bind to said repressor binding sequence thereby preventing transcription of the nucleic acid of interest. Said nucleic acid of interest may encode a protein and/or RNA of interest.

Repressor proteins that may be utilized in donor cells include, for example, those listed in Table 1. In an embodiment, the donor cell expresses a repressor protein selected from the repressor proteins listed in Table 1 and the nucleic acid of interest contained within the genetic circuit is under the transcriptional regulation of a repressor binding sequence bound by said repressor protein.

TABLE 1

| Repressor protein | SwissProt Accession number |
|---|---|
| AmeR | Q9F8V9 |
| AmrR | Q9RG61 |
| AmtR | Q9S3L4 |
| ArpA | Q54189 |
| ArpR | Q9KJC4 |
| BarA | Q9LBV6 |
| BarB | O24739 |
| BM1P1 | O68276 |
| BM3R1 | P43506 |
| BpeR | Q6VV70 |
| ButR | Q9AJ68 |
| CalR1 | Q8KNI9 |
| CampR | Q93TU7 |
| CasR | Q9F6W0 |
| CprB | O66129 |
| CymR | O33453 |
| Cyp106 | Q59213 |
| DhaR | Q9RAJ1 |
| Ef0113 | Q8KU49 |
| EthR | P96222 |
| FarA | O24741 |
| HapR | O30343 |
| HemR | P72185 |
| HlyllR | Q63B57 |
| IcaR | Q9RQQ0 |
| IcaR | Q8GLC6 |
| IfeR | O68442 |
| JadR2 | Q56153 |
| KstR | Q9RA03 |
| LanK | Q9ZGB7 |
| LitR | Q8KX64 |
| LmrA | O34619 |
| LuxT | Q9ANS7 |
| McbR | Q8NLK1 |
| MmfR | Q9JN89 |
| MtrR | P39897 |
| NonG | Q9XDF0 |
| OpaR | O50285 |
| Orf2 | Q9XDV7 |
| orfL6 | Q8VV87 |
| PaaR | Q9FA56 |
| PhlF | Q9RF02 |
| PqrA | Q9F147 |
| PsbI | Q9XDW2 |
| PsrA | Q9EX90 |
| Q9ZF45 | Q9ZF45 |
| QacR | P0A0N4 |
| RmrR | Q9KIH5 |
| ScbR | O86852 |
| SmcR | Q9L8G8 |
| SmeT | Q8KLP4 |
| SrpR | Q9R9T9 |
| TarA | Q9RPK9 |
| TcmR | P39885 |
| ThlR | O85706 |
| TtgR | Q9AIU0 |
| TtgW | Q93PU7 |
| TylP | Q9XCC7 |
| TylQ | Q9ZHP8 |
| UrdK | Q9RP98 |
| VanT | Q8VQC6 |
| VarR | Q9AJL5 |
| YdeS | P96676 |
| YDH1 | P22645 |
| YixD | P32398 |

Repressor binding sequences corresponding to such repressor proteins are well known in the art and the skilled person may easily choose functional pairs of repressor protein/repressor binding sequence. In some embodiments, the donor cell expresses one or several repressor proteins corresponding to one or several repressor binding sequences contained in the genetic circuit. The repressor protein(s) expressed by the donor cell and/or the repressor binding sequence(s) comprised on the genetic circuit may be heterologous to the donor cell, i.e. are not naturally present in said donor cell. In particular, the repressor protein(s) expressed by the donor cell and/or the repressor binding sequence(s) comprised on the genetic circuit may come from a different bacterial species that the donor cell, for example, from a different bacterial genus. In an embodiment, the repressor protein(s) expressed by the donor cell and/or the repressor binding sequence(s) comprised on the genetic circuit are endogenous to the donor cell, i.e. are naturally present in said donor cell.

In another embodiment, the repressor protein is a CRISPR nuclease devoid of nuclease activity. In this embodiment, the repressor protein is used in combination with a guide RNA targeting a sequence required for transcription of the nucleic acid of interest. In this embodiment, the donor cell thus also expresses said guide RNA in addition to the CRISPR nuclease acting as repressor protein. In particular, the guide RNA may target a control sequence such as the promoter, RBS or repressor binding sequence operably linked to the nucleic acid of interest. Alternatively, the guide RNA may target a non controlling sequence such as the coding region of the nucleic acid of interest. In this case, said non controlling sequence should be considered as the repressor binding sequence. Thanks to this guide RNA, the CRISPR nuclease devoid of nuclease activity is able to bind the targeted sequence without inducing any break, thereby preventing transcription of the nucleic acid of interest without altering the integrity of the genetic circuit. CRISPR nucleases devoid of nuclease activity such as dead Cas9 (dCas9) are well known by the skilled person.

In addition to expression of a repressor protein, the donor cell may further comprise a packaging prophage that provides in trans the necessary components for assembly of bacterial delivery vehicles, such as for example bacteriophage scaffold. Once produced, the delivery vehicles may be advantageously used to transfer the genetic circuit of interest into a recipient or target cell. The absence of repressor protein in the recipient or targeted cell results in expression of the transferred genetic circuit of interest.

As used herein, the term “genetic circuit”, for example, refers to a nucleic acid construct, for example, a linear or circular double stranded DNA molecule, comprising one or more nucleic acid of interest operably linked to control sequences including a transcriptional promoter (“promoter”) and a repressor binding sequence (recognized by at least one repressor protein expressed by the donor cell). The genetic circuit may further comprise one or more additional nucleic acid sequences operably linked to one or several control sequences including a transcriptional promoter. In particular, the genetic circuit may comprise one or more nucleic acid of interest operably linked to control sequences including a promoter and a repressor binding sequence (recognized by at least one repressor protein expressed by the donor cell), and one or more nucleic acid sequences operably linked to a constitutive promoter. For example, in a particular embodiment, the genetic circuit comprises a nucleic acid of interest encoding a CRISPR nuclease operably linked to control sequences including a promoter and a repressor binding sequence (recognized by at least one repressor protein expressed by the donor cell), and a nucleic acid sequence encoding a guide RNA operably linked to a constitutive promoter. The term “operably linked” means a configuration in which a control sequence is placed at an appropriate position relative to a nucleic acid of interest, in such a way that the control sequence directs expression of said nucleic acid. Optionally, the genetic circuit may include other control sequences such as leader sequence, polyadenylation sequence, propeptide sequence, ribozyme, hairpin-forming sequences, ribosome binding site, signal peptide sequence and/or transcription terminator. In some embodiments, the genetic circuit is a plasmid or a phagemid, i.e. comprises a genetic sequence that signals for packaging. In some embodiments, the genetic circuit is a phagemid.

In one aspect, methods of producing delivery vehicles using the disclosed donor cell lines are provided. As used herein, the term “delivery vehicle” refers for example, to any means that allows the transfer of a genetic circuit into a cell. In an embodiment, the delivery vehicle allows the transfer of a genetic circuit into a bacterial cell (“bacterial delivery vehicle”). In certain embodiments, the delivery vehicles are prepared by introducing the genetic circuit described herein into a suitable donor cell under conditions that permit formation of the delivery vehicles. For example, in certain embodiments, the method comprises (i) introducing into a donor cell the genetic circuit of interest; and (ii) allowing a sufficient amount of time for replication of the genetic circuit and packaging of the genetic circuit into the delivery vehicles. More particularly, the method of producing delivery vehicles may comprise (i) introducing into a donor cell as defined above, for example, a bacterial donor cell, comprising a genetic circuit of interest as defined above and prophage sequences encoding proteins required in trans for assembly of the genetic circuit into a delivery vehicle (e.g. bacteriophage scaffolding proteins); and (ii) allowing a sufficient amount of time for replication of the genetic circuit and packaging of the genetic circuit into the bacterial delivery vehicles. In one aspect, the method may further comprise the step of collection and, optionally, purification of the delivery vehicles.

In yet another aspect, donor cells are provided comprising a repressor protein expression cassette comprising sequences encoding repressor proteins. Donor cells may comprise a repressor protein expression cassette comprising a sequence encoding a repressor protein or several sequences encoding several repressor proteins. Further, in any of these embodiments, the donor cell may comprise prophage sequences encoding proteins required in trans for packaging of the nucleic acid payload of interest comprised in the genetic circuit (or the genetic circuit in its entirety) into a delivery vehicle. Such proteins include, for example, structural bacteriophage proteins, e.g., capsid proteins.

Methods are provided which enable transfer of a genetic circuit comprising a nucleic acid of interest encoding a protein or RNA molecule of interest, from a donor cell into a desired recipient or target cell. Genetic circuit and donor cell may be as defined above. In an embodiment, the target cell is a bacterial cell and does not express the repressor protein(s) found in the donor cell to negatively regulate the transcription of the nucleic acid of interest contained in the genetic circuit. Said methods may comprise contacting said donor cells with said recipient or target cells for a time sufficient for transfer of the genetic circuit. Alternatively, where bacterial delivery vehicles are produced (e.g. using a donor cell comprising a bacteriophage scaffold), the method may comprise contacting said recipient or target cells with the bacterial delivery vehicles produced in donor cells. The transferred genetic circuit comprises a nucleic acid of interest under the control of a repressor binding sequence that is negatively regulated by the donor cell expressed repressor protein. In such instances, transcription of the nucleic acid of interest encoding the protein or RNA molecule of interest is repressed through expression of a repressor protein in the donor cell. The methods provided herein enable transfer of a genetic circuit, comprising a nucleic acid encoding one or more proteins or RNA molecules of interest, into a desired target host cell which does not express, for example, naturally lacks, the repressor protein thereby allowing transcription of the payload of interest.

In certain embodiments, the nucleic acid of interest is selected from the group consisting of a nucleic acid encoding a RNA such as a mRNA, crRNA, tRNA, iRNA (interference RNA), asRNA (anti-sense RNA), ribozyme RNA, RNA aptamer or a guide RNA, a CRISPR locus, a toxin gene, a gene encoding an enzyme such as a nuclease or a kinase, a gene encoding a nuclease selected from the group consisting of a Cas nuclease, a Cas9 nuclease, a TALEN, a ZFN and a meganuclease, a gene encoding a recombinase, a bacterial receptor, a membrane protein, a structural protein or a secreted protein, a gene encoding resistance to an antibiotic or to a drug in general, a gene encoding a toxic protein or a toxic factor, and a gene encoding a virulence protein or a virulence factor, or any of their combination. The nucleic acid of interest may also encode a bacterial transporter or a bacterial pore or secretion system. Proteins encoded by the nucleic acid of interest can be modified or engineered to include extra features, like the addition or removal of a function (e.g. dCas9), the addition of a secretion signal to a protein not normally secreted or the addition of an exogenous peptide in a loop.

In an embodiment, the nucleic acid of interest encodes a therapeutic protein. In another embodiment, the nucleic acid of interest encodes an anti-sense nucleic acid molecule. In some embodiments, the nucleic acid of interest encodes two or more molecules of interest. In particular, one of these molecules may be a nuclease, for instance a Cas nuclease, and another molecule may be a nucleic acid molecule such as a guide RNA. In one aspect, the methods and compositions provided herein enable the transfer of a genetic circuit comprising a nucleic acid of interest that encodes a nuclease that targets cleavage of a host bacterial cell genome or a host bacterial cell plasmid. In some embodiments, the nuclease mediated cleavage occurs in an antibiotic resistant gene. In some other embodiments, the nuclease mediated cleavage of the host bacterial cell genome is designed to stimulate a homologous recombination event for insertion of a nucleic acid of interest into the genome of the bacterial cell.

Methods and compositions are provided which enable transfer of a genetic circuit comprising a nucleic acid of interest encoding a protein or RNA molecule of interest, into a desired target or recipient cell. As used herein, the term “transfer” refers to any means that allows the transfer of a genetic circuit into a recipient or target cell. Such means include, for example, transduction, conjugation and transformation. In some embodiments, delivery vehicles may be used to transfer genetic circuits from the donor cell to the target cell. Delivery vehicles encompassed by the present disclosure include, without limitation, bacteriophage scaffold, virus scaffold, chemical based delivery vehicle (e.g., cyclodextrin, calcium phosphate, cationic polymers, cationic liposomes), protein-based or peptide-based delivery vehicle, lipid-based delivery vehicle, nanoparticle-based delivery vehicles, non-chemical-based delivery vehicles (e.g., transformation, electroporation, sonoporation, optical transfection), particle-based delivery vehicles (e.g., gene gun, magnetofection, impalefection, particle bombardment, cell-penetrating peptides) or donor bacteria (conjugation). In preferred embodiments, delivery vehicles are bacteriophage scaffolds, i.e. obtained from natural, evolved or engineered capsids.

Any combination of delivery vehicles is also encompassed by the present disclosure. The delivery vehicle can refer to a bacteriophage derived scaffold and can be obtained from a natural, evolved or engineered capsid. In some embodiments, the delivery vehicle is the payload (e.g. genetic circuit) as bacteria are naturally competent to take up a payload from the environment on their own.

As used herein, the term “payload” refers to any one or more nucleic acid sequence, such as the genetic circuits disclosed herein, and/or amino acid sequence, or a combination of both (such as, without limitation, peptide nucleic acid or peptide-oligonucleotide conjugate) transferred into a recipient or target cell with a delivery vehicle. The term “payload” may also refer to a plasmid, a vector or a cargo. The payload can be a phagemid or phasmid obtained from natural, evolved or engineered bacteriophage genome. The payload can also be composed only in part of phagemid or phasmid obtained from natural, evolved or engineered bacteriophage genome.

As used herein, the term “nucleic acid” refers to a sequence of at least two nucleotides covalently linked together which can be single-stranded or double-stranded or contains portion of both single-stranded and double-stranded sequence. Nucleic acids as disclosed herein can be naturally occurring, recombinant or synthetic. The nucleic acid can be in the form of a circular sequence or a linear sequence or a combination of both forms. The nucleic acid can be DNA, both genomic or cDNA, or RNA or a combination of both. The nucleic acid may contain any combination of deoxyribonucleotides and ribonucleotides, and any combination of bases, including uracil, adenine, thymine, cytosine, guanine, inosine, xathanine, hypoxathanine, isocytosine, 5-hydroxymethylcytosine and isoguanine. Other examples of modified bases that can be used are detailed in Chemical Reviews 2016, 116 (20) 12655-12687. The term “nucleic acid” also encompasses any nucleic acid analogs which may contain other backbones comprising, without limitation, phosphoramide, phosphorothioate, phosphorodithioate, O-methylphophoroamidite linkage and/or deoxyribonucleotides and ribonucleotides nucleic acids. Any combination of the above features of a nucleic acid is also encompassed by the present disclosure.

The genetic circuits may also comprise an origin of replication. Origins of replication, for use in the disclosed methods and compositions, are known in the art and have been identified from species-specific plasmid DNAs (e.g. CoIE1, R1, pT181, pSC101, pMB1, R6K, RK2, p15a and the like), from bacterial virus (e.g. φX174, M13, F1 and P4) and from bacterial chromosomal origins of replication (e.g. oriC). Such sequences permit, for example, replication of the genetic circuit in a bacterial cell, e.g. a donor cell and/or targeted cell. In one embodiment, a phagemid (genetic circuit) according to the disclosure comprises a bacterial origin of replication that is functional in a donor, target or recipient cell.

Alternatively, the genetic circuit according to the disclosure does not comprise any functional bacterial origin of replication or contains an origin of replication that is inactive in the targeted bacteria. Thus, the genetic circuit of the disclosure cannot replicate by itself once it has been introduced into a target or recipient cell.

In one embodiment, the origin of replication on the genetic circuit, or a plasmid to be packaged into a delivery vehicle, is inactive in the targeted bacteria, meaning that this origin of replication is not functional in the targeted cell, thus preventing unwanted plasmid replication.

In one embodiment, the genetic circuit or plasmid comprises a bacterial origin of replication that is functional in the donor bacteria cell, e.g in the donor bacteria cell used for the production of the bacterial virus particles.

Genetic circuit or plasmid replication depends on host enzymes and on genetic circuit or plasmid-controlled cis and trans determinants. For example, some genetic circuits or plasmids may have determinants that are recognized in almost all gram-negative bacteria and act correctly in each host during replication initiation and regulation. Other genetic circuits or plasmids may possess this ability only in some bacteria (Kues, U and Stahl, U 1989 Microbiol Rev 53:491-516).

Genetic circuits or plasmids may be replicated by three general mechanisms, namely theta type, strand displacement, and rolling circle (reviewed by Del Solar et al. 1998 Microhio and Molec Biol. Rev 62:434-464) that start at the origin of replication. These replication origins contain sites that are required for interactions of genetic circuit or plasmid and/or host encoded proteins.

Origins of replication used herein may be of moderate copy number, such as colEl ori from pBR322 (15-20 copies per cell) or the R6K plasmid (15-20 copies per cell) or may be high copy number, e.g. pUC oris (500-700 copies per cell), pGEM oris (300-400 copies per cell), pTZ oris (>1000 copies per cell) or pBluescript oris (300-500 copies per cell).

In one embodiment, the bacterial origin of replication comprised in the genetic circuit is selected in the group consisting of ColE1, pMB1 and variants (pBR322, pET, pUC, etc), p15a, ColA, ColE2, pOSAK, pSC101, R6K, IncW (pSa etc), IncFII, pT181, P1, F IncP, IncC, IncJ, IncN, IncP1, IncP4, IncQ, IncH11, RSF1010, CloDF13, NTP16, R1, f5, pPS10, pC194, pE194, BBR1, pBC1, pEP2, pWVO1, pLF1311, pAP1, pWKS1, pLS1, pLS11, pUB6060, pJD4, pIJ101, pSN22, pAMbeta1, pIP501, pIP407, ZM6100(Sa), pCU1, RA3, pMOL98, RK2/RP4/RP1/R68, pB10, R300B, pRO1614, pRO1600, pECB2, pCM1, pFA3, RepFIA, RepFIB, RepFIC, pYVE439-80, R387, phasyl, RA1, TF-FC2, pMV158 and pUB113.

In non-limiting embodiments, the bacterial origin of replication is a E. coli origin of replication selected in the group consisting of ColE1, pMB1 and variants (pBR322, pET, pUC, etc), p15a, ColA, ColE2, pOSAK, pSC101, R6K, IncW(pSa etc), IncFII, pT181, P1, F IncP, IncC, IncJ, IncN, IncP1, IncP4, IncQ, IncH11, RSF1010, CloDF13, NTP16, R1, f5 and pPS10.

In non-limiting embodiments, the bacterial origin of replication is selected in the group consisting of pC194, pE194, BBR1, pBC1, pEP2, pWVO1, pLF1311, pAP1, pWKS1, pLS1, pLS11, pUB6060, pJD4, pIJ101, pSN22, pAMbeta1, pIP501, pIP407, ZM6100(Sa), pCU1, RA3, pMOL98, RK2/RP4/RP1/R68, pB10, R300B, pRO1614, pRO1600, pECB2, pCM1, pFA3, RepFIA, RepFIB, RepFIC, pYVE439-80, R387, phasyl, RA1, TF-FC2, pMV158 and pUB113.

In a specific embodiment, the bacterial origin of replication are ColE1 and p15A.

The genetic circuit may comprise a phage replication origin. In particular, the delivered nucleic acid sequences according to the disclosure may comprise a phage replication origin which can initiate, with complementation of a complete phage genome, the replication of the delivered nucleic acid sequence for later encapsulation into the different capsids. A phage origin of replication can also be engineered to act as a bacterial origin of replication without the need to package any phage particles.

A phage origin of replication comprised in the genetic circuit or in the delivered nucleic acid sequence of the disclosure can be any origin of replication found in a phage.

In an embodiment, the phage origin of replication can be the wild-type or non-wildtype sequence of the M13, f1, φX174, P4, lambda, P2, 186, lambda-like, HK022, mEP237, HK97, HK629, HK630, mEPO43, mEP213, mEP234, mEP390, mEP460, mEPx1, mEPx2, phi80, mEP234, T2, T4, T5, T7, RB49, phiX174, R17, PRD1 P1-like, P2-like, P22, P22-like, N15 and N15-like bacteriophages.

In an embodiment, the phage origin of replication is selected in the group consisting of phage origins of replication of M13, f1, φX174, P4, and lambda.

In a particular embodiment, the phage origin of replication is the lambda or P4 origin of replication.

The genetic circuit as disclosed herein comprises a nucleic acid sequence of interest under the transcriptional control of a repressor binding sequence. In the disclosed methods, transcription of the nucleic acid of interest is repressed in the donor cell while active in the targeted cell. In one embodiment, the nucleic acid of interest is a programmable nuclease circuit to be delivered to the targeted cell, i.e. the nucleic acid of interest encodes a programmable nuclease system to be delivered to the targeted cell. The programmable nuclease system comprises a programmable nuclease, i.e. a nuclease which is able to mediate sequence-specific elimination of a target gene of interest in the targeted cell. In particular, this programmable nuclease system may be able to mediate in vivo sequence-specific elimination of bacteria that contain a target gene of interest (e.g. a gene that is harmful to humans). Programmable nucleases which can be used in the methods disclosed herein include, for example, CRISPR nucleases (also named “CRISPR-associated proteins” or “Cas nucleases”), such as Type I, Type II CRISPR nucleases, TALEN nucleases, ZFN nucleases, meganucleases and recombinases and any variants thereof (evolved or engineered variants). These nucleases may be used separately or in combination, i.e. the programmable nuclease system may comprise one or several programmable nucleases. In a particular embodiment, the programmable nuclease is selected from engineered variants of the Type II CRISPR-Cas (Clustered Regularly Interspaced Short Palindromic Repeats-CRISPR-associated) system of Streptococcus pyogenes. Other programmable nucleases that can be used include other CRISPR-Cas systems, engineered TALEN (Transcription Activator-Like Effector Nuclease) variants, engineered zinc finger nuclease (ZFN) variants, natural, evolved or engineered meganuclease or recombinase variants, and any combination or hybrids of programmable nucleases. Depending on the type of nuclease used in the method, the programmable nuclease system may further comprise one or several additional components. In particular, when the programmable nuclease in a CRISPR nuclease, the programmable nuclease system further comprises, for example, a guide RNA to find and selectively cleave the targeted sequence. Thus, the engineered autonomously distributed nuclease circuits provided herein may be used to selectively cleave DNA encoding a gene of interest in the target cell such as, for example, a toxin gene, a virulence factor gene, an antibiotic resistance gene, a remodeling gene or a modulatory gene (cf. WO2014124226).

Other sequences of interest, for example, programmable sequences, can be included to the delivered nucleic acid sequence so as to be delivered to targeted cells. In an embodiment, the nucleic acid of interest encodes a molecule that affects the survival or the growth of the targeted cell, for example the target bacterium. In embodiments wherein the target cell is a bacterium, such a molecule may be choosen in order to lead to cell death (bactericidal effect) or to prevent the growth (bacteriostatic effect) of said bacterium. For example, the nucleic acid sequence of interest may encode holins, endolysins, restriction enzymes or toxins affecting the target cell, for example, affecting the survival or the growth of the target cell.

In a particular embodiment, the nucleic acid of interest encodes a bacteriocin. The bacteriocin can be a proteinaceous toxin produced by bacteria to kill or inhibit growth of other bacteria. Bacteriocins are categorized in several ways, including producing strain, common resistance mechanisms, and mechanism of killing. Such bacteriocin had been described from Gram negative bacteria (e.g. microcins, colicin-like bacteriocins and tailocins) and from Gram positive bacteria (e.g. Class I, Class II, Class III or Class IV bacteriocins). The nucleic acid of interest may also encode a transporter needed to secrete the toxin to the extracellular space.

In a more particular embodiment, the nucleic acid of interest comprises a sequence encoding a toxin selected in the group consisting of microcins, colicin-like bacteriocins, tailocins, Class I, Class II, Class III and Class IV bacteriocins.

In a particular embodiment, the corresponding immunity polypeptide (i.e. anti-toxin) may be used to protect bacterial cells (see review by Cotter et al., Nature Reviews Microbiology 11: 95, 2013, which is hereby incorporated by reference in its entirety) for delivered nucleic acid sequence production and encapsidation purpose but is absent in the pharmaceutical composition and in the targeted bacteria in which the nucleic acid of interest is delivered.

In some other embodiments, expression of the transferred nucleic acid of interest in the target cell does not lead to cell death. For example, the nucleic acid of interest may encode a reporter gene, e.g. leading to a luminescence or fluorescence signal.

In some other embodiments, the nucleic acid of interest may encode proteins, in particular enzymes, achieving a useful function in the target cell such as modifying the metabolism of the target cell, the composition of its environment or affecting the host comprising the target cell.

In a particular embodiment, the nucleic sequence of interest is selected in the group consisting of a nucleic acid encoding a RNA such as a mRNA, crRNA, tRNA, iRNA (interference RNA), asRNA (anti-sense RNA), ribozyme RNA, RNA aptamer or a guide RNA (gRNA), a CRISPR locus, a gene encoding an enzyme such as a nuclease or a kinase, a gene encoding a nuclease selected from the group consisting of a Cas nuclease, a Cas9 nuclease, a TALEN, a ZFN or a meganuclease, a gene encoding a recombinase, a bacterial receptor, a membrane protein, a structural protein, a secreted protein, or resistance to an antibiotic or to a drug in general, a gene a gene encoding a toxic protein or a toxic factor and a gene encoding a virulence protein, a virulence factor, a bacterial transporter or a bacterial pore, and any of their combinations. Proteins encoded by the nucleic acid of interest can also be modified or engineered to include extra features, like the addition or removal of a function (e.g. dCas9), the addition of a secretion signal to a protein not normally secreted, the addition of an exogenous peptide in a loop, etc . . .

In some embodiments, the nucleic acid of interest encodes a CRISPR system. Typically, a CRISPR system contains two distinct elements, i.e. i) an endonuclease, in this case the CRISPR associated nuclease (Cas or “CRISPR associated protein”) and ii) a guide RNA. The structure of the guide RNA may depend on the nature of the Cas nuclease. In particular, the guide RNA (gRNA ou sgRNA) may be in the form of a chimeric RNA which consists of the combination of a CRISPR (RNAcr) bacterial RNA and a RNAtracr (trans-activating RNA CRISPR) (Jinek et al., Science 2012). The gRNA combines the targeting specificity of the cRNA corresponding to the “spacing sequences” that serve as guides to the Cas proteins, and the conformational properties of the Rtracr in a single transcript. Such guide RNA is required for example when the Cas nuclease is Cas9.

Alternatively, the guide RNA may only comprise a RNAcr. Such guide RNA is required for example when the Cas nuclease is Cpf1. When the gRNA and the Cas protein are expressed simultaneously in the cell, the target genomic sequence can be permanently interrupted (and causing disappearance of the targeted and surrounding sequences and/or cell death, depending on the location) or modified. The modification may be guided by a repair matrix. In general, the CRISPR system includes two main classes depending on the nuclease mechanism of action. Class 1 is made of multi-subunit effector complexes and includes type I, III and IV. Class 2 is made of single-unit effector modules, like Cas9 nuclease, and includes type II (II-A,II-B,II-C,II-C variant), V (V-A,V-B,V-C,V-D,V-E,V-U1,V-U2,V-U3,V-U4,V-U5) and VI (VI-A,VI-B1,VI-B2,VI-C,VI-D)

The nucleic acid of interest according to the present disclosure may comprise a nucleic acid sequence encoding a Cas protein. A variety of CRISPR enzymes are available for use as a sequence of interest on the plasmid. In some embodiments, the CRISPR enzyme is a Type II CRISPR enzyme. In some embodiments, the CRISPR enzyme catalyzes DNA cleavage. In some other embodiments, the CRISPR enzyme catalyzes RNA cleavage. In one embodiment, the CRISPR enzyme may be coupled to a sgRNA. In certain embodiments, the sgRNA targets a gene selected in the group consisting of an antibiotic resistance gene, virulence protein or factor gene, toxin protein or factor gene, a bacterial receptor gene, a membrane protein gene, a structural protein gene, a secreted protein gene, a gene expressing resistance to a drug in general or a gene causing a deleterious effect to the host (a host comprising the target cell).

Non-limiting examples of Cas proteins as part of a multi-subunit effector or as a single-unit effector include Cas1, Cas1B, Cas2, Cas3, Cas4, Cas5, Cas6, Cas7, Cas8, Cas9 (also known as Csn1 and Csx12), Cas10, Cas11 (SS), Cas12a Φpf1), Cas12b (C2c1), Cas12c (C2c3), Cas12d (CasY), Cas12e (CasX), C2c4, C2c8, C2c5, C2c10, C2c9, Cas13a (C2c2), Cas13b (C2c6), Cas13c (C2c7), Cas13d, Csa5, Csc1, Csc2, Cse1, Cse2, Csy1, Csy2, Csy3, Csf1, Csf2, Csf3, Csf4, Csm2, Csm3, Csm4, Csm5, Csm6, Cmr1, Cmr3, Cmr4, Cmr5, Cmr6, Csn2, Csb1, Csb2, Csb3, Csx17, Csx14, Csx10, Csx16, CsaX, Csx13, Csx1, Csx15, SdCpf1, CmtCpf1, TsCpf1, CmaCpf1, PcCpf1, ErCpf1, FbCpf1, UbcCpf1, AsCpf1, LbCpf1, homologues thereof, orthologues thereof, variants thereof, or modified versions thereof. In some embodiments, the CRISPR enzyme cleaves both strands of the target nucleic acid at the Protospacer Adjacent Motif (PAM) site.

In a particular embodiment, the CRISPR enzyme is any Cas9 protein, for instance any naturally-occurring bacterial Cas9 as well as any variants, homologs or orthologs thereof.

By “Cas9” is meant a protein Cas9 (also called Csn1 or Csx12) or a functional protein, peptide or polypeptide fragment thereof, i.e. capable of interacting with the guide RNA(s) and of exerting the enzymatic activity (nuclease) which allows it to perform the double-strand cleavage of the DNA of the target genome. “Cas9” can thus denote a modified protein, for example truncated to remove domains of the protein that are not essential for the predefined functions of the protein, in particular the domains that are not necessary for interaction with the gRNA (s).

The sequence encoding Cas9 (the entire protein or a fragment thereof) as used in the context of the disclosure can be obtained from any known Cas9 protein (Fonfara et al., Nucleic Acids Res 42 (4), 2014; Koonin et al., Nat Rev Microbiol 15(3), 2017). Examples of Cas9 proteins useful in the present disclosure include, but are not limited to, Cas9 proteins of Streptococcus pyogenes (SpCas9), Streptococcus thermophiles (St1Cas9, St3Cas9), Streptococcus mutans, Staphylococcus aureus (SaCas9), Campylobacter jejuni (CjCas9), Francisella novicida (FnCas9) and Neisseria meningitides (NmCas9).

In another particular embodiment, the CRISPR enzyme is any Cas12a, Cas13a or Cas13d protein, for instance any naturally-occurring bacterial Cas12a, Cas13a or Cas13d as well as any variants, homologs or orthologs thereof.

The sequence encoding Cpf1 (Cas12a) (the entire protein or a fragment thereof) as used in the context of the disclosure can be obtained from any known Cpf1 (Cas12a) protein (Koonin et al., 2017). Examples of Cpf1(Cas12a) proteins useful in the present disclosure include, but are not limited to, Cpf1(Cas12a) proteins of Acidaminococcus sp, Lachnospiraceae bacteriu and Francisella novicida.

The sequence encoding Cas13a (the entire protein or a fragment thereof) can be obtained from any known Cas13a (C2c2) protein (Abudayyeh et al., 2017). Examples of Cas13a (C2c2) proteins useful in the present disclosure include, but are not limited to, Cas13a (C2c2) proteins of Leptotrichia wadei (LwaCas13a).

The sequence encoding Cas13d (the entire protein or a fragment thereof) can be obtained from any known Cas13d protein (Yan et al., 2018). Examples of Cas13d proteins useful in the present disclosure include, but are not limited to, Cas13d proteins of Eubacterium siraeum and Ruminococcus sp.

In a particular embodiment, the nucleic acid of interest encodes a CRISPR/Cas system, such as a CRISPR/Cas9 system, for the reduction of gene expression or inactivation of a gene selected from the group consisting of an antibiotic resistance gene, virulence factor or protein gene, toxin factor or protein gene, a gene expressing a bacterial receptor, a membrane protein, a structural protein, a secreted protein, a gene expressing resistance to a drug in general or a gene causing a deleterious effect to the host.

In one embodiment, the CRISPR system is used to target and inactivate a virulence factor. A virulence factor can be any substance produced by a pathogen that alter host-pathogen interaction by increasing the degree of damage done to the host. Virulence factors are used by pathogens in many ways, including, for example, in cell adhesion or colonization of a niche in the host, to evade the host's immune response, to facilitate entry to and egress from host cells, to obtain nutrition from the host, or to inhibit other physiological processes in the host. Virulence factors can include enzymes, endotoxins, adhesion factors, motility factors, factors involved in complement evasion, and factors that promote biofilm formation. For example, such targeted virulence factor gene can be E. coli virulence factor gene such as, without limitation, EHEC-HlyA, Stx1 (VT1), Stx2 (VT2), Stx2a (VT2a), Stx2b (VT2b), Stx2c (VT2c), Stx2d (VT2d), Stx2e (VT2e) and Stx2f (VT2f), Stx2h (VT2h), stx2k, fimA, fimF, fimH, neuC, kpsE, sfa, foc, iroN, aer, iha, papC, papGI, papGII, papGIII, hlyC, cnf1, hra, sat, ireA, usp ompT, ibeA, malX, fyuA, irp2, traT, afaD, ipaH, eltB, estA, bfpA, eaeA, espA, aaiC, aatA, TEM, CTX, SHV, csgA, csgB, csgC, csgD, esgE, csgF, csgG, csgH, TlSS, T2SS, T3SS, T4SS, T5SS, T6SS (secretion systems). For example, such targeted virulence factor gene can be Shigella dysenteriae virulence factor gene such as, without limitation, stx1 and stx2. For example, such targeted virulence factor gene can be Yersinia pestis virulence factor gene such as, without limitation, yscF (plasmid-borne (pCDl) T3SS external needle subunit). For example, such targeted virulence factor gene can be Francisella tularensis virulence factor gene such as, without limitation, fslA. For example, such targeted virulence factor gene can be Bacillus anthracis virulence factor gene such as, without limitation, pag (Anthrax toxin, cell-binding protective antigen). For example, such targeted virulence factor gene can be Vibrio cholera virulence factor gene such as, without limitation, ctxA and ctxB (cholera toxin), tcpA (toxin co-regulated pilus), and toxT (master virulence regulator). For example, such targeted virulence factor gene can be Pseudomonas aeruginosa virulence factor genes such as, without limitation, pyoverdine (e.g., sigma factor pvdS, biosynthetic genes pvdL, pvdl, pvdJ, pvdH, pvdA, pvdF, pvdQ, pvdN, pvdM, pvdO, pvdP, transporter genes pvdE, pvdR, pvdT, opmQ), siderophore pyochelin (e.g., pchD, pchC, pchB, pchA, pchE, pchF and pchG, and toxins (e.g., exoU, exoS and exoT). For example, such targeted virulence factor gene can be Klebsiella pneumoniae virulence factor genes such as, without limitation, fimA (adherence, type I fimbriae major subunit), and cps (capsular polysaccharide). For example, such targeted virulence factor gene can be Acinetobacter baumannii virulence factor genes such as, without limitation, ptk (capsule polymerization) and epsA (assembly). For example, such targeted virulence factor gene can be Salmonella enterica Typhi virulence factor genes such as, without limitation, MIA (invasion, SPI-1 regulator), ssrB (SPI-2 regulator), and those associated with bile tolerance, including efflux pump genes acrA, acrB and tolC. For example, such targeted virulence factor gene can be Fusobacterium nucleatum virulence factor genes such as, without limitation, FadA and TIGIT. For example, such targeted virulence factor gene can be Bacteroidesfragilis virulence factor genes such as, without limitation, bft.

In another embodiment, the CRISPR system, such as a CRISPR/Cas9 system, is used to target and inactivate an antibiotic resistance gene such as, without limitation, GyrB, ParE, ParY, AAC(1), AAC(2′), AAC(3), AAC(6′), ANT(2″), ANT(3″), ANT(4′), ANT(6), ANT(9), APH(2″), APH(3″), APH(3′), APH(4), APH(6), APH(7″), APH(9), ArmA, RmtA, RmtB, RmtC, Sgm, AER, BLA1, CTX-M, KPC, SHV, TEM, BlaB, CcrA, IMP, NDM, VIM, ACT, AmpC, CMY, LAT, PDC, OXA β-lactamase, mecA, Omp36, OmpF, PIB, bla (blaI, blaR1) and mec (mecI, mecR1) operons, Chloramphenicol acetyltransferase (CAT), Chloramphenicol phosphotransferase, Ethambutol-resistant arabinosyltransferase (EmbB), MupA, MupB, Integral membrane protein MprF, Cfr 23S rRNA methyltransferase, Rifampin ADP-ribosyltransferase (Arr), Rifampin glycosyltransferase, Rifampin monooxygenase, Rifampin phosphotransferase, DnaA, RbpA, Rifampin-resistant beta-subunit of RNA polymerase (RpoB), Erm 23S rRNA methyltransferases, Lsa, MsrA, Vga, VgaB, Streptogramin Vgb lyase, Vat acetyltransferase, Fluoroquinolone acetyltransferase, Fluoroquinolone-resistant DNA topoisomerases, Fluoroquinolone-resistant GyrA, GyrB, ParC, Quinolone resistance protein (Qnr), FomA, FomB, FosC, FosA, FosB, FosX, VanA, VanB, VanD, VanR, VanS, Lincosamide nucleotidyltransferase (Lin), EreA, EreB, GimA, Mgt, Ole, Macrolide phosphotransferases (MPH), MefA, MefE, Mel, Streptothricin acetyltransferase (sat), Su11, Su12, Sul3, sulfonamide-resistant FolP, Tetracycline inactivation enzyme TetX, TetA, TetB, TetC, Tet30, Tet31, TetM, TetO, TetQ, Tet32, Tet36, MacAB-TolC, MsbA, MsrA,VgaB, EmrD, EmrAB-TolC, NorB, GepA, MepA, AdeABC, AcrD, MexAB-OprM, mtrCDE, EmrE, adeR, acrR, baeSR, mexR, phoPQ, mtrR, or any antibiotic resistance gene described in the Comprehensive Antibiotic Resistance Database (CARD https://card.mcmaster.ca/).

In another embodiment, the CRISPR system, such as a CRISPR/Cas9 system, is used to target and inactivate a bacterial toxin gene. Bacterial toxin can be classified as either exotoxins or endotoxins. Exotoxins are generated and actively secreted; endotoxins remain part of the bacteria.

The response to a bacterial toxin can involve severe inflammation and can lead to sepsis. Such toxin can be for example Botulinum neurotoxin, Tetanus toxin, Staphylococus toxins, Diphteria toxin, Anthrax toxin, Alpha toxin, Pertussis toxin, Shiga toxin, Heat-stable enterotoxin (E. coli ST), colibactin, BFT (B. fragilis toxin) or any toxin described in Henkel et al., (Toxins from Bacteria in EXS. 2010; 100: 1-29).

The terms “donor cell”, “target cell” and “recipient cell” as used herein refers, for example, to prokaryotic cells, such as bacterial cells. In particular, the “donor”, “target” or “recipient” cells disclosed herein can be any bacteria present or that can be present in a mammal organism. It can be any commensal, symbiotic or pathogenic bacteria of the microbiota or microbiome.

A microbiome may comprise of a variety of endogenous bacterial species, any of which may be targeted in accordance with the present disclosure. In some embodiments, where bacterial delivery vehicles are used, the genus and/or species of targeted endogenous bacterial cells may depend on the type of bacteriophages being used for preparing the bacterial delivery vehicles. For example, some bacteriophages exhibit tropism for, or preferentially target, specific host species of bacteria. Other bacteriophages do not exhibit such tropism and may be used to target a number of different genus and/or species of endogenous bacterial cells.

Examples of bacterial cells that can be used as donor cells or target cells include, without limitation, cells from bacteria of the genus Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Vibrio spp., Bacillus spp., Erysipelothrix spp., Salmonella spp., Streptomyces spp., Streptococcus spp., Staphylococcus spp., Bacteroides spp., Prevotella spp., Clostridium spp., Bifidobacterium spp., Clostridium spp., Brevibacterium spp., Lactococcus spp., Leuconostoc spp., Actinobacillus spp., Selnomonas spp., Shigella spp., Zymonas spp., Mycoplasma spp., Treponema spp., Leuconostoc spp., Corynebacterium spp., Enterococcus spp., Enterobacter spp., Pyrococcus spp., Serratia spp., Morganella spp., Parvimonas spp., Fusobacterium spp., Actinomyces spp., Porphyromonas spp., Micrococcus spp., Bartonella spp., Borrelia spp., Brucelia spp., Campylobacter spp., Chlamydophilia spp., Cutibacterium spp., Propionibacterium spp., Gardnerella spp., Ehrlichia spp., Haemophilus spp., Leptospira spp., Listeria spp., Mycoplasma spp., Nocardia spp., Rickettsia spp., Ureaplasma spp., and Lactobacillus spp., and a mixture thereof.

In an embodiment, the bacteria can be selected from the group consisting of Yersinia spp., Escherichia spp., Klebsiella spp., Acinetobacter spp., Pseudomonas spp., Helicobacter spp., Vibrio spp, Salmonella spp., Streptococcus spp., Staphylococcus spp., Bacteroides spp., Clostridium spp., Shigella spp., Enterococcus spp., Enterobacter spp., Listeria spp., Cutibacterium spp., Propionibacterium spp., Fusobacterium spp., Porphyromonas spp. And Gardnerella spp.

In some embodiments, bacterial cells of the present disclosure that can be used as donor cells or target cells are anaerobic bacterial cells (e.g., cells that do not require oxygen for growth).

Anaerobic bacterial cells include facultative anaerobic cells such as but not limited to Escherichia coli, Shewanella oneidensis, Gardnerella vaginalis and Listeria. Anaerobic bacterial cells also include obligate anaerobic cells such as, for example, Bacteroides, Clostridium, Cutibacterium, Propionibacterium, Fusobacterium and Porphyromona species. In humans, anaerobic bacteria are most commonly found in the gastrointestinal tract. In some particular embodiments, the targeted bacteria (target cells) are thus bacteria most commonly found in the gastrointestinal tract. Bacterial delivery vehicles, such as for example bacteriophages used for preparing the bacterial virus particles, and then the bacterial virus particles, may target (e.g., to specifically target) anaerobic bacterial cells according to their specific spectra known by the person skilled in the art to specifically deliver the plasmid.

In some embodiments, the bacterial cells that can be used as donor cells or target cells are, without limitation, Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphilococcus lugdunensis, Leuconostoc oenos, Corynebacterium xerosis, Lactobacillus plantarum, Lactobacillus rhamnosus, Lactobacillus casei, Lactobacillus acidophilus, Enterococcus faecalis, Bacillus coagulans, Bacillus cereus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Streptomyces phaechromogenes, Streptomyces ghanaenis, Klebsiella pneumoniae, Enterobacter cloacae, Enterobacter aerogenes, Serratia marcescens, Morganella morganii, Citrobacter freundii, Propionibacterium freudenreichii, Pseudomonas aerigunosa, Parvimonas micra, Prevotella intermedia, Fusobacterium nucleatum, Prevotella nigrescens, Actinomyces israelii, Porphyromonas endodontalis, Porphyromonas gingivalis Micrococcus luteus, Bacillus megaterium, Aeromonas hydrophila, Aeromonas caviae, Bacillus anthracis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Campylobacter coli, Campylobacter fetus, Chlamydia pneumoniae, Chlamydia trachomatis, Chlamydophila psittaci, Clostridium botulinum, Clostridium difficile, Clostridium perfringens, Clostridium tetani, Corynebacterium diphtheria, Cutibacterium acnes (formerly Propionibacterium acnes), Ehrlichia canis, Ehrlichia chaffeensis, Enterococcus faecium, Francisella tularensis, Haemophilus influenza, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumonia, Neisseria gonorrhoeae, Neisseria meningitides, Nocardia asteroids, Rickettsia rickettsia, Salmonella enteritidis, Salmonella typhi, Salmonella paratyphi, Salmonella typhimurium, Shigella flexnerii, Shigella dysenteriae, Staphylococcus saprophyticus, Streptococcus pneumoniae, Streptococcus pyogenes, Gardnerella vaginalis, Streptococcus viridans, Treponema pallidum, Ureaplasma urealyticum, Vibrio cholera, Vibrio parahaemolyticus, Yersinia pestis, Yersinia enterocolitica, Yersinia pseudotuberculosis, Actinobacter baumanii, Pseudomonas aerigunosa, and a mixture thereof. In an embodiment, the bacterial cells that can be used as donor cells or target cells are selected from the group consisting of Escherichia coli, Enterococcus faecium, Staphylococcus aureus, Klebsiella pneumoniae, Acinetobacter baumanii, Pseudomonas aeruginosa, Enterobacter cloacae, and Enterobacter aerogenes, and a mixture thereof.

In one embodiment, the bacteria that can be used as donor cells or target cells are Escherichia coli.

In one embodiment, the bacteria that can be used as donor cells or target cells are pathogenic bacteria. The bacteria can be virulent bacteria.

The bacteria that can be used as donor cells or target cells can be antibacterial resistance bacteria, such as those selected from the group consisting of extended-spectrum beta-lactamase-producing (ESBL) Escherichia coli, ESBL Klebsiella pneumoniae, vancomycin-resistant Enterococcus (VRE), methicillin-resistant Staphylococcus aureus (MRSA), multidrug-resistant (MDR) Acinetobacter baumannii, MDR Enterobacter spp., and a combination thereof. In an embodiment, the bacteria can be selected from the group consisting of extended-spectrum beta-lactamase-producing (ESBL) Escherichia coli strains.

In some particular embodiments, the donor cell and/or the target cell is a probiotic. As used herein, the term “probiotic” includes, but is not limited to, bacterlactobacilli, bifidobacteria, streptococci, enterococci, propionibacteria, saccaromycetes, lactobacilli, bifidobacteria, or proteobacteria.

Alternatively, the bacterium that can be used as donor cell or target cell can be a bacterium of the microbiome of a given species, such as a bacterium of the human microbiota.

In some embodiments, the donor cell and the target cell are phylogenetically close, for example, of the same phylum, class, order, family, genus or species. In an embodiment, the donor cell and the target cell are of the same genus or species.

In certain embodiments, the present disclosure is directed to methods for transfer of a genetic circuit from a donor cell into a target or recipient cell through the production of bacterial delivery vehicles containing the genetic circuit as described herein. The bacterial delivery vehicles can be prepared from bacterial virus. The bacterial delivery vehicles are chosen in order to be able to introduce the genetic circuit into the targeted bacterial cell. The bacterial delivery vehicles may be engineered to target specific bacteria (see, for example, Ser. Application Nos. 62/771,761; 62/802,777; and 62/783,258 each of which is incorporated herein in their entirety)

In an embodiment, bacterial viruses, from which the bacterial delivery vehicles may be derived, are bacteriophages. Optionally, the bacteriophage is selected from the Order Caudovirales consisting of, based on the taxonomy of Krupovic et al, Arch Virol, 2015:

Bacteriophages may be selected from the family Myoviridae (such as, without limitation, genus Cp220virus, Cp8virus, Ea214virus, Felixolvirus, Mooglevirus, Suspvirus, Hp1virus, P2virus, Kayvirus, P100virus, Silviavirus, Spolvirus, Tsarbombavirus, Twortvirus, Cc31virus, Jd8virus, Js98virus, Kp15virus, Moonvirus, Rb49virus, Rb69virus, S16virus, Schizot4virus, Sp18virus, T4virus, Cr3virus, Selvirus, V5virus, Abouovirus, Agatevirus, Agrican357virus, Ap22virus, Arv1virus, B4virus, Bastillevirus, Bc431virus, Bcep78virus, Bcepmuvirus, Biquartavirus, Bxz1virus, Cd119virus, Cp51virus, Cvm10virus, Eah2virus, Elvirus, Hapunavirus, Jimmervirus, Kpp10virus, M12virus, Machinavirus, Marthavirus, Msw3virus, Muvirus, Myohalovirus, Nit1virus, P1virus, Pakpunavirus, Pbunavirus, Phikzvirus, Rheph4virus, Rs12virus, Rslunavirus, Secunda5virus, Sep1virus, Spn3virus, Svunavirus, Tg1virus, Vhm1virus and Wphvirus)

Bacteriophages may be selected from the family Podoviridae (such as, without limitation, genus Fri1virus, Kp32virus, Kp34virus, Phikmvvirus, Pradovirus, Sp6virus, T7virus, Cp1virus, P68virus, Phi29virus, Nona33virus, Pocjvirus, T12011virus, Bcep22virus, Bpp1virus, Cba41virus, Dfl12virus, Ea92virus, Epsilon15virus, F116virus, G7cvirus, Jwalphavirus, Kf1virus, Kpp25virus, Lit1virus, Luz24virus, Luz7virus, N4virus, Nonanavirus, P22virus, Pagevirus, Phieco32virus, Prtbvirus, Sp58virus, Una961virus and Vp5virus)

Bacteriophages may be selected from the family Siphoviridae (such as, without limitation, genus Camvirus, Likavirus, R4virus, Acadianvirus, Coopervirus, Pg1virus, Pipefishvirus, Rosebushvirus, Brujitavirus, Che9cvirus, Hawkeyevirus, Plotvirus, Jerseyvirus, K1gvirus, Sp31virus, Lmd1virus, Una4virus, Bongovirus, Reyvirus, Buttersvirus, Charlievirus, Redivirus, Baxtervirus, Nymphadoravirus, Bignuzvirus, Fishburnevirus, Phayoncevirus, Kp36virus, Rogue1virus, Rtpvirus, T1virus, T1svirus, Ab18virus, Amigovirus, Anatolevirus, Andromedavirus, Attisvirus, Barnyardvirus, Bernal13virus, Biseptimavirus, Bronvirus, C2virus, C5virus, Cba181virus, Cbastvirus, Cecivirus, Che8virus, Chivirus, Cjw1virus, Corndogvirus, Cronusvirus, D3112virus, D3virus, Decurrovirus, Demosthenesvirus, Doucettevirus, E125virus, Eiauvirus, Ff47virus, Gaiavirus, Gilesvirus, Gordonvirus, Gordtnkvirus, Harrisonvirus, Hk578virus, Hk97virus, Jenstvirus, Jwxvirus, Kelleziovirus, Korravirus, L5virus, lambdavirus, Laroyevirus, Liefievirus, Marvinvirus, Mudcatvirus, N15virus, Nonagvirus, Np1virus, Omegavirus, P12002virus, P12024virus, P23virus, P70virus, Pa6virus, Pamx74virus, Patiencevirus, Pbi1virus, Pepy6virus, Pfr1virus, Phic31virus, Phicbkvirus, Phietavirus, Phifelvirus, Phij11virus, Pis4avirus, Psavirus, Psimunavirus, Rdj1virus, Rer2virus, Sap6virus, Send513virus, Septima3virus, Seuratvirus, Sextaecvirus, Sfi11virus, Sfi21dt1virus, Sitaravirus, Sk1virus, Slashvirus, Smoothievirus, Soupsvirus, Spbetavirus, Ssp2virus, T5virus, Tankvirus, Tin2virus, Titanvirus, Tm4virus, Tp21virus, Tp84virus, Triavirus, Trigintaduovirus, Vegasvirus, Vendettavirus, Wbetavirus, Wildcatvirus, Wizardvirus, Woesvirus, Xp10virus, Ydn12virus and Yuavirus)

Bacteriophages may be selected from the family Ackermannviridae (such as, without limitation, genus Ag3virus, Limestonevirus, Cba120virus and Vi1virus)

Optionally, the bacteriophage is not part of the order Caudovirales but from families with unassigned order such as, without limitation, family Tectiviridae (such as genus Alphatectivirus, Betatectivirus), family Corticoviridae (such as genus Corticovirus), family Inoviridae (such as genus Fibrovirus, Habenivirus, Inovirus, Lineavirus, Plectrovirus, Saetivirus, Vespertiliovirus), family Cystoviridae(such as genus Cystovirus), family Leviviridae(such as genus Allolevivirus, Levivirus), family Microviridae (such as genus Alpha3microvirus, G4microvirus, Phix174microvirus, Bdellomicrovirus, Chlamydiamicrovirus, Spiromicrovirus) and family Plasmaviridae (such as genus Plasmavirus).

Optionally, the bacteriophage is targeting Archea not part of the Order Caudovirales but from families with Unassigned order such as, without limitation, Ampullaviridae, FuselloViridae, Globuloviridae, Guttaviridae, Lipothrixviridae, Pleolipoviridae, Rudiviridae, Salterprovirus and Bicaudaviridae.

A non-exhaustive listing of bacterial genera and their known host-specific bacteria viruses is presented in the following paragraphs. The bacterial delivery vehicles disclosed herein may be engineered, as non-limiting examples, from the following phages. Synonyms and spelling variants are indicated in parentheses. Homonyms are repeated as often as they occur (e.g., D, D, d).

Unnamed phages are indicated by “NN” beside their genus and their numbers are given in parentheses.

Bacteria of the genus Actinomyces can be infected by the following phages: Av-I, Av-2, Av-3, BF307, CT1, CT2, CT3, CT4, CT6, CT7, CT8 and 1281.

Bacteria of the genus Aeromonas can be infected by the following phages: AA-I, Aeh2, N, PM1, TP446, 3, 4, 11, 13, 29, 31, 32, 37, 43, 43-10T, 51, 54, 55R.1, 56, 56RR2, 57, 58, 59.1, 60, 63, Aeh1, F, PM2, 1, 25, 31, 40RR2.8t, (syn=44R), (syn=44RR2.8t), 65, PM3, PM4, PM5 and PM6.

Bacteria of the genus Bacillus can be infected by the following phages: A, aiz1, A1-K-I, B, BCJA1, BC1, BC2, BLL1, BL1, BP142, BSL1, BSL2, BS1, BS3, BS8, BS15, BS18, BS22, BS26, BS28, BS31, BS104, BS105, BS106, BTB, B1715V1, C, CK-I, Co11, Cor1, CP-53, CS-I, Csi, D, D, D, D5, ent1, FP8, FP9, Fsi, FS2, FS3, FS5, FS8, FS9, G, GH8, GT8, GV-I, GV-2, GT-4, g3, g12, g13, g14, g16, g17, g21, g23, g24, g29, H2, ken1, KK-88, Kum1, Kyu1, J7W-1, LP52, (syn=LP-52), L7, Mex1, MJ-I, mor2, MP-7, Mp10, MP12, MP14, MP15, Neo1, N°2, N5, N6P, PBC1, PBLA, PBP1, P2, S-a, SF2, SF6, Sha1, Si11, SP02, (syn=(ΦSPP1), SPβ, STI, Sti, SU-I1, t, TbI, Tb2, Tb5, TbIO, Tb26, Tb51, Tb53, Tb55, Tb77, Tb97, Tb99, Tb560, Tb595, Td8, Td6, Td15, Tg1, Tg4, Tg6, Tg7, Tg9, TgIO, Tg11, Tg13, Tg15, Tg21, Tin1, Tin7, Tin8, Tin13, Tm3, Toc1, Tog1, to11, TP-I, TP-10vir, TP-15c, TP-16c, TP-17c, TP-19, TP35, TP51, TP-84, Tt4, Tt6, type A, type B, type C, type D, type E, Tφ3, VA-9, W, wx23, wx26, Yun1, α, γ, p1 1,, φmed-2, φT, φμ-4, φ3T, φ75, φ1O5, (syn=φ1O5), IA, IB, 1-97A, 1-97B, 2, 2, 3, 3, 3, 5, 12, 14, 20, 30, 35, 36, 37, 38, 41C, 51, 63, 64, 138D, I, II, IV, NN-Bacillus (13), ale1, Ar1, AR2, AR3, AR7, AR9, Bace-11, (syn=11), Bastille, BL1, BL2, BL3, BL4, BLS, BL6, BL8, BL9, BP124, BS28, BS80, Ch, CP-51, CP-54, D-5, dar1, den1, DP-7, ent1, FoSi, FoS2, FS4, FS6, FS7, G, gall, gamma, Gel, GF-2, Gsi, GT-I, GT-2, GT-3, GT-4, GT-5, GT-6, GT-7, GV-6, g15, 19, 110, Isi, K, MP9, MP13, MP21, MP23, MP24, MP28, MP29, MP30, MP32, MP34, MP36, MP37, MP39, MP40, MP41, MP43, MP44, MP45, MP47, MP50, NLP-I, No. 1, N17, N19, PBS1, PK1, PMB1, PMB12, PMJ1, S, SPO1, SP3, SP5, SP6, SP7, SP8, SP9, Sp1O, SP-15, SP50, (syn=SP-50), SP82, SST, sub1, SW, Tg8, Tg12, Tg13, Tg14, thu1, thuA, thuS, Tin4, Tin23, TP-13, TP33, TP50, TSP-I, type V, type VI, V, Vx, β22, φe, φNR2, φ25, φ63, 1, 1, 2, 2C, 3NT, 4, 5, 6, 7, 8, 9, 10, 12, 12, 17, 18, 19, 21, 138, III, 4 (B. megateriwn), 4 (B. sphaericus), AR13, BPP-IO, BS32, BS107, Bi, B2, GA-I, GP-IO, GV-3, GV-5, g8, MP20, MP27, MP49, Nf, PP5, PP6, SF5, Tg18, TP-I, Versailles, φ15, φ²⁹, 1-97, 837/IV, mï-Bacillus (1), Bat1O, BSL1O, BSLI 1, BS6, BSI 1, BS16, BS23, Bs1O1, BS102, g18, mor1, PBL1, SN45, thu2, thu3, TmI, Tm2, TP-20, TP21, TP52, type F, type G, type IV, HN-BacMus (3), BLE, (syn=θc), BS2, BS4, BS5, BS7, B10, B12, BS20, BS21, F, MJ-4, PBA12, AP50, AP50-04, AP50-11, AP50-23, AP50-26, AP50-27 and Bam35. The following Bacillus-specific phages are defective: DLP10716, DLP-11946, DPB5, DPB12, DPB21, DPB22, DPB23, GA-2, M, No. IM, PBLB, PBSH, PBSV, PBSW, PBSX, PBSY, PBSZ, phi, Spa, type 1 and μ.

Bacteria of the genus Bacteroides can be infected by the following phages: crAss-phage, ad I2, Baf-44, Baf-48B, Baf-64, Bf-I, Bf-52, B40-8, F1, β1, φA1, φBrO1, (φBrO2, 11, 67.1, 67.3, 68.1, mt-Bacteroides (3), Bf42, Bf71, HN-Bdellovibrio (1) and BF-41.

Bacteria of the genus Bordetella can be infected by the following phages: 134 and NN-Bordetella (3).

Bacteria of the genus Borrellia can be infected by the following phages: NN-Borrelia (1) and NN-Borrelia (2).

Bacteria of the genus Brucella can be infected by the following phages: A422, Bk, (syn=Berkeley), BM29, Foi, (syn=Fo1), (syn=FQ1), D, FP2, (syn=FP2), (syn=FD2), Fz, (syn=Fz75/13), (syn=Firenze 75/13), (syn=Fi), Fi, (syn=F1), Fim, (syn=Fim), (syn=Fim), FiU, (syn=F1U), (syn=FiU), F2, (syn=F2), F3, (syn=F3), F4, (syn=F4), F5, (syn=F5), F6, F7, (syn=F7), F25, (syn=F25), (syn=£25), F25U, (syn=F25u), (syn=F25U), (syn=F25V), F44, (syn-F44), F45, (syn=F45), F48, (syn=F48), I, Im, M, MC/75, M51, (syn=M85), P, (syn=D), S708, R, Tb, (syn=TB), (syn=Tbilisi), W, (syn=Wb), (syn=Weybridge), X, 3, 6, 7, 10/1, (syn=10), (syn=F8), (syn=F8), 12m, 24/11, (syn=24), (syn=F9), (syn=F9), 45/111, (syn=45), 75, 84, 212/XV, (syn=212), (syn=Fi0), (syn=F1O), 371/XXIX, (syn=371), (syn=Fn), (syn=F1 1) and 513.

Bacteria of the genus Burkholderia can be infected by the following phages: CP75, NN-Burkholderia (1) and 42.

Bacteria of the genus Campylobacter can be infected by the following phages: C type, NTCC12669, NTCC12670, NTCC12671, NTCC12672, NTCC12673, NTCC12674, NTCC12675, NTCC12676, NTCC12677, NTCC12678, NTCC12679, NTCC12680, NTCC12681, NTCC12682, NTCC12683, NTCC12684, 32f, 111c, 191, NN-Campylobacter (2), Vfi-6, (syn=V19), VfV-3, V2, V3, V8, V16, (syn=Vfi-1), V19, V20(V45), V45, (syn=V-45) and NN-Campylobacter (1).

Bacteria of the genus Chlamydia can be infected by the following phage: Chp1.

Bacteria of the genus Clostridium can be infected by the following phages: CAK1, CA5, Ca7, Ceβ, (syn=1C), Ceγ, C1d1, c-n71, c-203 Tox-, Deβ, (syn=ID), (syn=1Dt0X+), HM3, KM1, KT, Ms, Na1, (syn=Naltox+), PA1350e, Pfó, PL73, PL78, PL81, P1, P50, P5771, P19402, 1Ct0X+, 2Ct0X 2D3 (syn=2Dt0X+), 3C, (syn=3Ct0x+), 4C, (syn=4Ct0X+), 56, III-1, NN-Clostridium (61), NB1t0X+, α1, Ca1, HMT, HM2, PF15 P-23, P-46, Q-05, Q-oe, Q-16, Q-21, Q-26, Q-40, Q-46, S111, SA02, WA01, WA03, Wm, W523, 80, C, CA2, CA3, CPT1, CPT4, c1, c4, c5, HM7, H11/A1, H18/Ax, FWS23, Hi58ZA1, K2ZA1, K21ZS23, ML, NA2tOX; Pf2, Pf3, Pf4, S9ZS3, S41ZA1, S44ZS23, α2, 41, 112ZS23, 214/S23, 233/Ai, 234/S23, 235/S23, II-1, II-2, II-3, NN-Clostridium (12), Cal, F1, K, S2, 1, 5 and NN-Clostridium (8).

Bacteria of the genus Corynebacterium can be infected by the following phages: CGK1 (defective), A, A2, A3, A1O1, A128, A133, A137, A139, A155, A182, B, BF, B17, B18, B51, B271, B275, B276, B277, B279, B282, C, capi, CC1, CG1, CG2, CG33, CL31, Cog, (syn=CG5), D, E, F, H, H-I, hqi, hq2, 11ZH33, Ii/31, J, K, K, (syn=Ktox”), L, L, (syn=Ltox+), M, MC-I, MC-2, MC-3, MC-4, MLMa, N, O, ovi, ov2, ov3, P, P, R, RP6, RS29, S, T, U, UB1, ub2, UH1, UH3, uh3, uh5, uh6, β, (syn=βtox+), βhv64, βvir, γ, (syn=γtoχ−), γ19, δ, (syn=δ′ox+), p, (syn=ptoχ-), (Φ9, (φ984, ω, IA, 1/1180, 2, 2/1180, 5/1180, 5ad/9717, 7/4465, 8/4465, 8ad/10269, 10/9253, 13Z9253, 15/3148, 21/9253, 28, 29, 55, 2747, 2893, 4498 and 5848.

Bacteria of the genus Enterococcus are infected by the following phages: DF78, F1, F2, 1, 2, 4, 14, 41, 867, D1, SB24, 2BV, 182, 225, C2, C2F, E3, E62, DS96, H24, M35, P3, P9, Sb1O1, S2, 2B1II, 5, 182a, 705, 873, 881, 940, 1051, 1057, 21096C, NN-Enterococcus (1), Pe1, F1, F3, F4, VD13, 1, 200, 235 and 341.

Bacteria of the genus Erysipelothrix can be infected by the following phage: NN-Erysipelothrix (1).

Bacteria of the genus Escherichia can be infected by the following phages: BW73, B278, D6, D108, E, E1, E24, E41, FI-2, FI-4, FI-5, HI8A, Ff18B, i, MM, Mu, (syn=mu), (syn=MuI), (syn=Mu-I), (syn=MU-I), (syn=MuI), (syn=μ), 025, PhI-5, Pk, PSP3, P1, P1D, P2, P4 (defective), S1, Wφ, φK13, φR73 (defective), φ1, φ2, φ7, φ92, ψ(defective), 7 A, 8φ, 9φ, 15 (defective), 18, 28-1, 186, 299, HH-Escherichia (2), AB48, CM, C4, C16, DD-VI, (syn=Dd-Vi), (syn=DDVI), (syn=DDVi), E4, E7, E28, f11, F13, H, Hi, H3, H8, K3, M, N, ND-2, ND-3, ND4, ND-5, ND6, ND-7, Ox-I (syn=oX1), (syn=HF), Ox-2 (syn=0x2), (syn=0X2), Ox-3, Ox-4, Ox-5, (syn=0X5), Ox-6, (syn=66F), (syn=φ66t), (syn=φ66t-)5 0111, PhI-I, RB42, RB43, RB49, RB69, S, Sal-I, Sal-2, Sal-3, Sal-4, Sal-5, Sal-6, TC23, TC45, TuII*-6, (syn=TuII*), TuIP-24, TuII*46, TuIP-60, T2, (syn=ganuTia), (syn=γ), (syn=PC), (syn=P.C.), (syn=T-2), (syn=T2), (syn=P4), T4, (syn=T-4), (syn=T4), T6, T35, α1, 1, IA, 3, (syn=Ac3), 3A, 3T+, (syn=3), (syn=Ml), 5φ, (syn=φ5), 9266Q, CFO103, HK620, J, K, KiF, m59, no. A, no. E, no. 3, no. 9, N4, sd, (syn=Sd), (syn=SD), (syn=Sa)3 (syn=sd), (syn=SD), (syn=CD), T3, (syn=T-3), (syn=T3), T7, (syn=T-7), (syn=T7), WPK, W31, ΔH, φC3888, φK3, φK7, φK12, φV-1, Φ04-CF, ψ05, ψ06, ψ07, φ1, φ1.2, φ20, φ95, φ263, φ1O92, φ1, φ11, (syn=φW), Ω8, 1, 3, 7, 8, 26, 27, 28-2, 29, 30, 31, 32, 38, 39, 42, 933W, NN-Escherichia (1), Esc-7-11, AC30, CVX-5, C1, DDUP, eC1, EC2, E21, E29, F1, F26S, F27S, Hi, HK022, HK97, (syn=(ΦHK97), HK139, HK253, HK256, K7, ND-I, no.D, PA-2, q, S2, T1, (syn=α), (syn=P28), (syn=T-I), (syn=Tx), T3C, T5, (syn=T-5), (syn=T5), UC-I, w, β4, γ2, λ, (syn=lambda), (syn=Φλ), ΦD326, φγ, ΦD06, Φ7, (10, φ80, χ, (syn=χi), (syn=Φχ), (syn=Φχi), 2, 4, 4A, 6, 8A, 102, 150, 168, 174, 3000, AC6, AC7, AC28, AC43, AC50, AC57, AC81, AC95, HK243, K1O, ZG/3A, 5, 5A, 21EL, H19-J, 933H, 0157 typing phages 1 to 16, JES-2013, 121Q, 172-1, 1720a-02, ADB-2, AKVF33, av-05, bV_EcoS_AHP42, bV_EcoS_AHP24, bC_EcoS_AHS24, bV_EcoS_AKS96 and CBA120.

Bacteria of the genus Fusobacterium are infected by the following phages: NN-Fusobacterium (2), fv83-554/3, fv88-531/2, 227, fv2377, fv2527 and fv8501.

Bacteria of the genus Haemophilus are infected by the following phages: HP1, S2 and N3.

Bacteria of the genus Helicobacter are infected by the following phages: HP1 and\hat{ }\hat{ }-Helicobacter (1).

Bacteria of the genus Klebsiella are infected by the following phages: AIO-2, KI4B, K16B, K19, (syn=K19), K114, K115, K121, K128, K129, K132, K133, K135, K1106B, K1171B, K1181B, K1832B, AIO-I, AO-I, AO-2, AO-3, FC3-10, K, K11, (syn=k11), K12, (syn=K12), K13, (syn=K13), (syn=K1 70/11), K14, (syn=K14), K15, (syn=K15), K16, (syn=K16), K17, (syn=K17), K18, (syn=K18), K119, (syn=K19), K127, (syn=K127), K131, (syn=K131), K135, K1171B, II, VI, IX, CI-I, K14B, K18, K111, K112, K113, K116, K117, K118, K120, K122, K123, K124, K126, K130, K134, K1106B, kIi65B, K1328B, KLXI, K328, P5046, 11, 380, III, IV, VII, VIII, FC3-11, K12B, (syn=K12B), K125, (syn=K125), K142B, (syn=K142), (syn=K142B), K1181B, (syn=k11 81), (syn=K1181B), K1765/!, (syn=K1765/1), K1842B, (syn=K1832B), K1937B, (syn=K1937B), L1, φ28, 7, 231, 483, 490, 632 and 864/100.

Bacteria of the genus Lepitospira are infected by the following phages: IE1, LE3, LE4 and ˜NN-Leptospira (1).

Bacteria of the genus Listeria are infected by the following phages: A511, 01761, 4211, 4286, (syn=B054), A005, A006, A020, A500, A502, A511, A1 18, A620, A640, B012, B021, B024, B025, B035, B051, B053, B054, B055, B056, B1O1, B11O, B545, B604, B653, C707, D441, HS047, H1OG, H8/73, H19, H21, H43, H46, H107, H108, HI 1O, H163/84, H312, H340, H387, H391/73, H684/74, H924A, PSA, U153, φMLUP5, (syn=P35), 00241, 00611, 02971A, 02971C, 5/476, 5/911, 5/939, 5/11302, 5/11605, 5/11704, 184, 575, 633, 699/694, 744, 900, 1090, 1317, 1444, 1652, 1806, 1807, 1921/959, 1921/11367, 1921/11500, 1921/11566, 1921/12460, 1921/12582, 1967, 2389, 2425, 2671, 2685, 3274, 3550, 3551, 3552, 4276, 4277, 4292, 4477, 5337, 5348/11363, 5348/11646, 5348/12430, 5348/12434, 10072, 11355C, 11711A, 12029, 12981, 13441, 90666, 90816, 93253, 907515, 910716 and NN-Lisferia (15).

Bacteria of the genus Morganella are infected by the following phage: 47.

Bacteria of the genus Mycobacterium are infected by the following phages: 13, aG1, aLi, ATCC 11759, A2, B.C3, BG2, BK1, BKS, butyricum, B-I, B5, B7, B30, B35, Clark, C1, C2, DNAIII, DSP1, D4, D29, GS4E, (syn=GS4E), GS7, (syn=GS-7), (syn=GS7), iPa, lacticola, Legendre, Leo, L5, (syn=ΦL-5), MC-I, MC-3, MC-4, minetti, MTPHI 1, Mx4, MyF3P/59a, phlei, (syn=phlei 1), phlei 4, Polonus II, rabinovitschi, smegmatis, TM4, TM9, tM1O, TM20, Y7, Y1O, φ630, IB, IF, IH, 1/1, 67, 106, 1430, BI, (syn=Bo1), B24, D, D29, F-K, F-S, HP, Polonus I, Roy, R1, (syn=R1-Myb), (syn=Ri), 11, 31, 40, 50, 103a, 103b, 128, 3111-D, 3215-D and NN-Mycobacterium (1).

Bacteria of the genus Neisseria are infected by the following phages: Group I, group II and NP1.

Bacteria of the genus Nocardia are infected by the following phages: MNP8, NJ-L, NS-8, N5 and TtiN-Nocardia.

Bacteria of the genus Proteus are infected by the following phages: Pm5, 13vir, 2/44, 4/545, 6/1004, 13/807, 20/826, 57, 67b, 78, 107/69, 121, 9/0, 22/608, 30/680, PmI, Pm3, Pm4, Pm6, Pm7, Pm9, PmIO, PmI 1, Pv2, π1, φm, 7/549, 9B/2, 10A/31, 12/55, 14, 15, 16/789, 17/971, 19A/653, 23/532, 25/909, 26/219, 27/953, 32A/909, 33/971, 34/13, 65, 5006M, 7480b, VI, 13/3a, Clichy 12, 7π2600, φχ7, 1/1004, 5/742, 9, 12, 14, 22, 24/860, 2600/D52, Pm8 and 24/2514.

Bacteria of the genus Providencia are infected by the following phages: PL25, PL26, PL37, 9211/9295, 9213/921 Ib, 9248, 7/R49, 7476/322, 7478/325, 7479, 7480, 9000/9402 and 9213/921 Ia.

Bacteria of the genus Pseudomonas are infected by the following phages: PfI, (syn=Pf-I), Pf2, Pf3, PP7, PRR1, 7s, im-Pseudomonas (1), AI-I, AI-2, B 17, B89, CB3, Col 2, Col 11, Col 18, Col 21, C154, C163, C167, C2121, E79, F8, ga, gb, H22, K1, M4, N2, Nu, PB-I, (syn=PB1), pf16, PMN17, PP1, PP8, Psa1, PsP1, PsP2, PsP3, PsP4, PsPS, PS3, PS17, PTB80, PX4, PX7, PYO1, PYO2, PYO5, PYO6, PYO9, PYO1O, PYO13, PYO14, PYO16, PYO18, PYO19, PYO20, PY029, PY032, PY033, PYO35, PY036, PY037, PY038, PY039, PYO41, PY042, PYO45, PY047, PY048, PY064, PY069, PYO103, P1K, SLP1, SL2, S2, UNL-I, wy, Yai, Ya4, Yan, YBE, φCTX, φC17, φKZ, (syn=ΦKZ), φ-LT, Φmu78, φNZ, φPLS-1, φST-1, φW-14, φ-2, 1/72, 2/79, 3, 3/DO, 4/237, 5/406, 6C, 6/6660, 7, 7v, 7/184, 8/280, 9/95, 10/502, 11/DE, 12/100, 12S, 16, 21, 24, 25F, 27, 31, 44, 68, 71, 95, 109, 188, 337, 352, 1214, HN-Pseudomonas (23), A856, B26, CI-I, CI-2, C5, D, gh-1, F1 16, HF, H90, K5, K6, K104, K109, K166, K267, N4, N5, 06N-25P, PE69, Pf, PPN25, PPN35, PPN89, PPN91, PP2, PP3, PP4, PP6, PP7, PP8, PP56, PP87, PP 114, PP206, PP207, PP306, PP651, Psp231a, Pssy401, Pssy9220, psi, PTB2, PTB20, PTB42, PX1, PX3, pX1O, PX12, PX14, PYO70, PYO71, R, SH6, SH133, tf, Ya5, Ya7, φBS, (Kf77, φ-MC, (mnF82, φPLS27, φPLS743, φS-1, 1, 2, 2, 3, 4, 5, 6, 7, 7, 8, 9, 10, 11, 12, 12B, 13, 14, 15, 14, 15, 16, 17, 18, 19, 20, 20, 21, 21, 22, 23, 23, 24, 25, 31, 53, 73, 119x, 145, 147, 170, 267, 284, 308, 525, NN-Pseudomonas (5), af, A7, B3, B33, B39, BI-I, C22, D3, D37, D40, D62, D3112, F7, F1O, g, gd, ge, gξHw12, Jb 19, KF1, L°, OXN-32P, 06N-52P, PCH-I, PC13-1, PC35-1, PH2, PH51, PH93, PH132, PMW, PM13, PM57, PM61, PM62, PM63, PM69, PM105, PM1 13, PM681, PM682, P04, PP1, PP4, PPS, PP64, PP65, PP66, PP71, PP86, PP88, PP92, PP401, PP711, PP891, Pssy41, Pssy42, Pssy403, Pssy404, Pssy420, Pssy923, PS4, PS-IO, Pz, SDI, SL1, SL3, SL5, SM, φC5, φC1 1, φC1 1-1, φC13, φC15, φMO, φX, φ04, φ1 1, φ240, 2, 2F, 5, 7m, 11, 13, 13/441, 14, 20, 24, 40, 45, 49, 61, 73, 148, 160, 198, 218, 222, 236, 242, 246, 249, 258, 269, 295, 297, 309, 318, 342, 350, 351, 357-1, 400-1, HN-Pseudomonas (6), G101, M6, M6a, L1, PB2, Pssy15, Pssy4210, Pssy4220, PYO12, PY034, PY049, PY050, PYO51, PY052, PY053, PY057, PY059, PY0200, PX2, PX5, SL4, φ03, φ06 and 1214.

Bacteria of the genus Rickettsia are infected by the following phage: NN-Rickettsia.

Bacteria of the genus Salmonella are infected by the following phages: b, Beccles, CT, d, Dundee, f, FeIs 2, GI, GUI, GVI, GVIII, k, K, i, j, L, 01, (syn=0-1), (syn=01), (syn=O-I), (syn=7), 02, 03, P3, P9a, P1O, Sab3, Sab5, San1S, San17, SI, Taunton, ViI, (syn=ViI), 9, imSalmonella (1), N-I, N-5, N-IO, N-17, N-22, 11, 12, 16-19, 20.2, 36, 449C/C178, 966A/C259, a, B.A.O.R., e, G4, GUI, L, LP7, M, MG40, N-18, PSA68, P4, P9c, P22, (syn=P22), (syn=PLT22), (syn=PLT22), P22a1, P22-4, P22-7, P22-11, SNT-I, SNT-2, SP6, Villi, ViIV, ViV, ViVI, ViVII, Worksop, Sj5, ε34, 1,37, 1(40), (syn=φ1-[40]), 1,422, 2, 2.5, 3b, 4, 5, 6,14(18), 8, 14(6,7), 10, 27, 28B, 30, 31, 32, 33, 34, 36, 37, 39, 1412, SNT-3, 7-11, 40.3, c, C236, C557, C625, C966N, g, GV, G5, G173, h, IRA, Jersey, MB78, P22-1, P22-3, P22-12, Sab1, Sab2, Sab2, Sab4, San1, San2, San3, San4, San6, San7, San8, San9, San13, San14, San16, San18, San19, San20, San21, San22, San23, San24, San25, San26, SasL1, SasL2, SasL3, SasL4, SasL5, S1BL, SII, ViII, φ1, 1, 2, 3a, 3a1, 1010, Ym-Salmonella (1), N-4, SasL6 and 27.

Bacteria of the genus Serratia are infected by the following phages: A2P, PS20, SMB3, SMP, SMP5, SM2, V40, V56, ic, ΦP-3, ΦP-6, 3M, 10/la, 20A, 34CC, 34H, 38T, 345G, 345P, 501B, SMB2, SMP2, BC, BT, CW2, CW3, CW4, CW5, Lt232, L2232, L34, L.228, SLP, SMPA, V.43, σ, φCW1, ΦCP6-1, ΦCP6-2, ΦCP6-5, 3T, 5, 8, 9F, 10/1, 20E, 32/6, 34B, 34CT, 34P, 37, 41, 56, 56D, 56P, 60P, 61/6, 74/6, 76/4, 101/8900, 226, 227, 228, 229F, 286, 289, 290F, 512, 764a, 2847/10, 2847/10a, L.359 and SMB1.

Bacteria of the genus Shigella are infected by the following phages: Fsa, (syn=a), FSD2d, (syn=D2d), (syn=W2d), FSD2E, (syn=W2e), fv, F6, f7.8, H-Sh, PES, P90, SfII, Sh, SHm, SHrv, (syn=HIV), sHvi, (syn=HVI), SHVvm, (syn=HVIII), SKγ66, (syn=gamma 66), (syn=γββ), (syn=γ66b), SKm, (syn=SIIIb)5 (syn=UI), SKw, (syn=Siva), (syn=IV), SIC™, (syn=SIVA.), (syn=IVA), sKvi, (syn=KVI), (syn=Svi), (syn=VI), SKvm, (syn=Svm), (syn=VIII), SKVIIIA, (syn=SvmA), (syn=VIIIA), sTvi, STK, STx1, STxn, S66, W2, (syn=D2c), (syn=D20), φ1, φIVb 3-SO-R, 8368-SO-R, F7, (syn=FS7), (syn=K29), F1O, (syn=fS1O), (syn=K31), I1, (syn=alfa), (syn=fSa), (syn=K1 8), (syn=α), I2, (syn=a), (syn=K19), SG33, (syn=G35), (syn=SO-35/G), SG35, (syn=SO-55/G), SG3201, (syn=SO-3201/G), SHn, (syn=HII), SHv, (syn=SHV), SHx, SHX, SKn, (syn=K2), (syn=KII), (syn=Sn), (syn=SsII), (syn=II), SKrv, (syn=Sm), (syn=SsIV), (syn=IV), SK1Va, (syn=Swab), (syn=SsIVa), (syn=iVa), SKV, (syn=K4), (syn=KV), (syn=SV), (syn=SsV), (syn=V), SKx, (syn=K9), (syn=KX), (syn=SX), (syn=SsX), (syn=X), STV, (syn=T35), (syn=35-50-R), STvm, (syn=T8345), (syn=8345-SO—S-R), W1, (syn=D8), (syn=FSD8), W2a, (syn=D2A), (syn=FS2a), DD-2, Sf6, fSi, (syn=F1), SF6, (syn=F6), SG42, (syn=SO-42/G), SG3203, (syn=SO-3203/G), SKF12, (syn=SsF12), (syn=F12), (syn=F12), STn, (syn=1881-SO-R), γ66, (syn=gamma 66a), (syn=Ssγ66), φ2, bI1, DDVII, (syn=DD7), FSD2b, (syn=W2B), FS2, (syn=F2), (syn=F2), FS4, (syn=F4), (syn=F4), FS5, (syn=F5), (syn=F5), FS9, (syn=F9), (syn=F9), F1 1, P2-SO-S, SG36, (syn=SO-36/G), (syn=G36), SG3204, (syn=SO-3204/G), SG3244, (syn=SO-3244/G), sHi, (syn=HI), SHvπ, (syn=HVII), SHK, (syn=HIX), SHx1, SHx7r, (syn=HXn), SKI, KI, (syn=S1), (syn=SsI), SKVII, (syn=KVII), (syn=Svπ), (syn=SsVII), SKIX, (syn=KIX), (syn=S1x), (syn=SsIX), SKXII, (syn=KXII), (syn=Sxn), (syn=SsXII), sTi, STffl, STrv, STVi, STvπ, 570, 5206, U2-SO-S, 3210-SO-S, 3859-SO-S, 4020-SO-S, φ3, φ5, φ7, φ8, φ9, φ1O, φ1 1, φ13, φ14, φ18, SHm, (syn=Hπi), sHχi, (syn=HXt) and sKxI, (syn=KXI), (syn=Sχi), (syn=SsXI), (syn=XI).

Bacteria of the genus Staphylococcus are infected by the following phages: A, EW, K, Ph5, Ph9, PhIO, Ph13, P1, P2, P3, P4, P8, P9, P1O, RG, SB-i, (syn=Sb-I), S3K, Twort, ΦSK311, φ812, 06, 40, 58, 119, 130, 131, 200, 1623, STC1, (syn=stc1), STC2, (syn=stc2), 44AHJD, 68, aC1, AC2, A6“C”, A9“C”, b581, CA-I, CA-2, CA-3, CA-4, CA-5, DI1, L39x35, L54a, M42, Ni, N2, N3, N4, N5, N7, N8, N1O, Ni 1, N12, N13, N14, N16, Ph6, Ph12, Ph14, UC-18, U4, U15, S1, S2, S3, S4, S5, X2, Z1, φB5-2, φD, ω, 11, (syn=φ1 1), (syn=P11-M15), 15, 28, 28A, 29, 31, 31B, 37, 42D, (syn=P42D), 44A, 48, 51, 52, 52A, (syn=P52A), 52B, 53, 55, 69, 71, (syn=P71), 71A, 72, 75, 76, 77, 79, 80, 80a, 82, 82A, 83 A, 84, 85, 86, 88, 88A, 89, 90, 92, 95, 96, 102, 107, 108, 111, 129-26, 130, 130A, 155, 157, 157A, 165, 187, 275, 275A, 275B, 356, 456, 459, 471, 471A, 489, 581, 676, 898, 1139, 1154A, 1259, 1314, 1380, 1405, 1563, 2148, 2638A, 2638B, 2638C, 2731, 2792A, 2792B, 2818, 2835, 2848A, 3619, 5841, 12100, AC3, A8, A1O, A13, b594n, D, HK2, N9, N15, P52, P87, S1, S6, Z4, pRE, 3A, 3B, 3C, 6, 7, 16, 21, 42B, 42C, 42E, 44, 47, 47A5 47C, 51, 54, 54x1, 70,73,75,78, 81, 82, 88, 93, 94, 101, 105, 110, 115, 129/16, 174, 594n, 1363/14, 2460 and mS-Staphylococcus (1).

Bacteria of the genus Streptococcus are infected by the following phages: EJ-I, NN-Streptococais (1), a, C1, FL0Ths, H39, Cp-I, Cp-5, Cp-7, Cp-9, Cp-IO, AT298, A5, a1O/Ji, a1O/J2, a1O/J5, a1O/J9, A25, BTI1, b6, cA1, c20-1, c20-2, DP-I, Dp-4, DT1, ET42, e1O, FA101, FEThs, Fκ. FKKIOI, FKLIO, FKP74, FKH, FLOThs, FyIO1, f1, F10, F20140/76, g, GT-234, HB3, (syn=HB-3), HB-623, HB-746, M102, 01205, p01205, PST, PO, P1, P2, P3, P5, P6, P8, P9, P9, P12, P13, P14, P49, P50, P51, P52, P53, P54, P55, P56, P57, P58, P59, P64, P67, P69, P71, P73, P75, P76, P77, P82, P83, P88, sc, sch, sf, SfI1 1, (syn=sFi1 1), (syn=φSFi11), (syn=φSfi1 1), (syn=φSfi1 1), sfi19, (syn=sFi19), (syn=φSFi19), (syn=φSfi19), Sfi21, (syn=sFi21), (syn=φSFi21), (syn=φSfi21), ST0, STX, st2, ST2, ST4, S3, (syn=φS3), s265, Φ17, φ42, Φ57, φ80, φ81, φ82, φ83, φ84, φ85, φ86, φ87, φ88, φ89, φ90, φ91, φ92, φ93, φ94, φ95, φ96, φ97, φ98, φ99, φ1OO, φ1O1, φ102, φ227, Φ7201, ω1, ω2, ω3, ω4, ω5, ω6, ω8, ω1o, 1, 6, 9, 1OF, 12/12, 14, 17SR, 195, 24, 50/33, 50/34, 55/14, 55/15, 70/35, 70/36, 71/ST15, 71/45, 71/46, 74F, 79/37, 79/38, 80/J4, 80/J9, 80/ST16, 80/15, 80/47, 80/48, 101, 103/39, 103/40, 121/41, 121/42, 123/43, 123/44, 124/44, 337/ST17 and mStreptococcus (34).

Bacteria of the genus Treponema are infected by the following phage: NN-Treponema (1).

Bacteria of the genus Vibrio are infected by the following phages: CTX(P, fs, (syn=si), fs2, Ivpf5, Vf12, Vf33, VPIΦ, VSK, v6, 493, CP-T1, ET25, kappa, K139, Labo1, )XN-69P, OXN-86, 06N-21P, PB-I, P147, rp-1, SE3, VA-I, (syn=VcA-I), VcA-2, VP1, VP2, VP4, VP7, VP8, VP9, vP1O, VP17, VP18, VP19, X29, (syn=29 d'Herelle), t, ΦHAWI-1, ΦHAWI-2, ΦHAWI-3, ΦHAWI-4, ΦHAWI-5, ΦHAWI-6, ΦHAWI-7, ΦHAWI-8, ΦHAWI-9, ΦHAWI-10, (HCl-1, ΦHC1-2, ΦHC1-3, ΦHC1-4, ΦHC2-1, >HC2-2, ΦHC2-3, ΦHC2-4, ΦHC3-1, φHC3-2, ΦHC3-3, ΦHD1S-1, ΦHD1S-2, ΦHD2S-1, ΦHD2S-2, ΦHD2S-3, ΦHD2S-4, ΦHD2S-5, φHDO-1, ΦHDO-2, ΦHDO-3, ΦHDO-4, ΦHDO-5, ΦHDO-6, ΦKL-33, ΦKL-34, ΦKL-35, ΦKL-36, ΦKWH-2, ΦKWH-3, ΦKWH-4, ΦMARQ-1, ΦMARQ-2, ΦMARQ-3, ΦMOAT-1, (P0139, ΦPEL1A-1, ΦPEL1A-2, ΦPEL8A-1, ΦPEL8A-2, ΦPEL8A-3, ΦPEL8C-1, ΦPEL8C-2, ΦPEL13A-1, ΦPEL13B-1, ΦPEL13B-2, ΦPEL13B-3, ΦPEL13B-4, ΦPEL13B-5, ΦPEL13B-6, ΦPEL13B-7, ΦPEL13B-8, ΦPEL13B-9, ΦPEL13B-10, φVP143, φVP253, φ16, φ138, 1-II, 5, 13, 14, 16, 24, 32, 493, 6214, 7050, 7227, II, (syn=group II), (syn==φ2), V, VIII, ˜m-Vibrio (13), KVP20, KVP40, nt-1, 06N-22P, P68, e1, e2, e3, e4, e5, FK, G, I, K, nt-6, N1, N2, N3, N4, N5, 06N-34P, OXN-72P, OXN-85P, OXN-100P, P, Ph-I, PL163/10, Q, S, T, φ92, 1-9, 37, 51, 57, 70A-8, 72A-4, 72A-10, 110A-4, 333, 4996, I (syn=group I), III (syn=group III), VI, (syn=A-Saratov), VII, IX, X, HN-Vibrio (6), pA1, 7, 7-8, 70A-2, 71A-6, 72A-5, 72A-8, 108A-10, 109A-6, 109A-8, 1 1OA-1, 110A-5, 110A-7, hv-1, OXN-52P, P13, P38, P53, P65, P108, Pill, TP13 VP3, VP6, VP12, VP13, 70A-3, 70A-4, 70A-10, 72A-1, 108A-3, 109-B1, 110A-2, 149, (syn=(149), IV, (syn=group IV), NN-Vibrio (22), VP5, VP11, VP15, VP16, a1, α2, α3a, α3b, 353B and HN-Vibrio (7).

Bacteria of the genus Yersinia are infected by the following phages: H, H-1, H-2, H-3, H-4, Lucas 110, Lucas 303, Lucas 404, YerA3, YerA7, YerA20, YerA41, 3/M64-76, 5/G394-76, 6/C753-76, 8/C239-76, 9/F18167, 1701, 1710, PST, 1/F2852-76, D′Herelle, EV, H, Kotljarova, PTB, R, Y, YerA41, φYerO3-12, 3, 4/C1324-76, 7/F783-76, 903, 1/M6176 and Yer2AT.

In an embodiment, the bacteriophage used as bacterial delivery vehicle is selected in the group consisting of Salmonella virus SKML39, Shigella virus AG3, Dickeya virus Limestone, Dickeya virus RC2014, Escherichia virus CBA120, Escherichia virus PhaxI, Salmonella virus 38, Salmonella virus Det7, Salmonella virus GG32, Salmonella virus PM10, Salmonella virus SFP10, Salmonella virus SH19, Salmonella virus SJ3, Escherichia virus ECML4, Salmonella virus Marshall, Salmonella virus Maynard, Salmonella virus SJ2, Salmonella virus STML131, Salmonella virus ViI, Erwinia virus Ea2809, Klebsiella virus 0507KN21, Serratia virus IME250, Serratia virus MAM1, Campylobacter virus CP21, Campylobacter virus CP220, Campylobacter virus CPt10, Campylobacter virus IBB35, Campylobacter virus CP81, Campylobacter virus CP30A, Campylobacter virus CPX, Campylobacter virus NCTC12673, Erwinia virus Ea214, Erwinia virus M7, Escherichia virus AYO145A, Escherichia virus EC6, Escherichia virus HY02, Escherichia virus JH2, Escherichia virus TP1, Escherichia virus VpaE1, Escherichia virus wV8, Salmonella virus FelixO1, Salmonella virus HB2014, Salmonella virus Mushroom, Salmonella virus UAB87, Citrobacter virus Moogle, Citrobacter virus Mordin, Escherichia virus SUSP1, Escherichia virus SUSP2, Aeromonas virus phiO18P, Haemophilus virus HP1, Haemophilus virus HP2, Pasteurella virus F108, Vibrio virus K139, Vibrio virus Kappa, Burkholderia virus phi52237, Burkholderia virus phiE122, Burkholderia virus phiE202, Escherichia virus 186, Escherichia virus P4, Escherichia virus P2, Escherichia virus Wphi, Mannheimia virus PHL101, Pseudomonas virus phiCTX, Ralstonia virus RSA1, Salmonella virus Fels2, Salmonella virus PsP3, Salmonella virus SopEphi, Yersinia virus L413C, Staphylococcus virus G1, Staphylococcus virus G15, Staphylococcus virus JD7, Staphylococcus virus K, Staphylococcus virus MCE2014, Staphylococcus virus P108, Staphylococcus virus Rodi, Staphylococcus virus S253, Staphylococcus virus S25-4, Staphylococcus virus SA12, Listeria virus A511, Listeria virus P100, Staphylococcus virus Remus, Staphylococcus virus SA11, Staphylococcus virus Stau2, Bacillus virus Camphawk, Bacillus virus SPO1, Bacillus virus BCP78, Bacillus virus TsarBomba, Staphylococcus virus Twort, Enterococcus virus phiEC24C, Lactobacillus virus Lb338-1, Lactobacillus virus LP65, Enterobacter virus PG7, Escherichia virus CC31, Klebsiella virus JD18, Klebsiella virus PKO111, Escherichia virus Bp7, Escherichia virus IME08, Escherichia virus JS10, Escherichia virus JS98, Escherichia virus QLO1, Escherichia virus VR5, Enterobacter virus Eap3, Klebsiella virus KP15, Klebsiella virus KP27, Klebsiella virus Matisse, Klebsiella virus Miro, Citrobacter virus Merlin, Citrobacter virus Moon, Escherichia virus JSE, Escherichia virus phi1, Escherichia virus RB49, Escherichia virus HXO1, Escherichia virus JSO9, Escherichia virus RB69, Shigella virus UTAM, Salmonella virus 516, Salmonella virus STML198, Vibrio virus KVP40, Vibrio virus nt1, Vibrio virus ValKK3, Escherichia virus VR7, Escherichia virus VR20, Escherichia virus VR25, Escherichia virus VR26, Shigella virus SP18, Escherichia virus AR1, Escherichia virus C40, Escherichia virus E112, Escherichia virus ECML134, Escherichia virus HY01, Escherichia virus ImeO9, Escherichia virus RB3, Escherichia virus RB14, Escherichia virus T4, Shigella virus Pss1, Shigella virus Shf12, Yersinia virus D1, Yersinia virus PST, Acinetobacter virus 133, Aeromonas virus 65, Aeromonas virus Aeh1, Escherichia virus RB16, Escherichia virus RB32, Escherichia virus RB43, Pseudomonas virus 42, Cronobacter virus CR3, Cronobacter virus CR8, Cronobacter virus CR9, Cronobacter virus PBESO2, Pectobacterium virus phiTE, Cronobacter virus GAP31, Escherichia virus 4MG, Salmonella virus SE1, Salmonella virus SSE121, Escherichia virus FFH2, Escherichia virus FV3, Escherichia virus JES2013, Escherichia virus V5, Brevibacillus virus Abouo, Brevibacillus virus Davies, Bacillus virus Agate, Bacillus virus Bobb, Bacillus virus Bp8pC, Erwinia virus Deimos, Erwinia virus Ea35-70, Erwinia virus RAY, Erwinia virus Simmy50, Erwinia virus SpecialG, Acinetobacter virus AB1, Acinetobacter virus AB2, Acinetobacter virus AbC62, Acinetobacter virus AP22, Arthrobacter virus ArV1, Arthrobacter virus Trina, Bacillus virus AvesoBmore, Bacillus virus B4, Bacillus virus Bigbertha, Bacillus virus Riley, Bacillus virus Spock, Bacillus virus Troll, Bacillus virus Bastille, Bacillus virus CAM003, Bacillus virus Bc431, Bacillus virus Bcp1, Bacillus virus BCP82, Bacillus virus BM15, Bacillus virus Deepblue, Bacillus virus JBP901, Burkholderia virus Bcep1, Burkholderia virus Bcep43, Burkholderia virus Bcep781, Burkholderia virus BcepNY3, Xanthomonas virus OP2, Burkholderia virus BcepMu, Burkholderia virus phiE255, Aeromonas virus 44RR2, Mycobacterium virus Alice, Mycobacterium virus Bxz1, Mycobacterium virus Dandelion, Mycobacterium virus HyRo, Mycobacterium virus 13, Mycobacterium virus Nappy, Mycobacterium virus Sebata, Clostridium virus phiC2, Clostridium virus phiCD27, Clostridium virus phiCD119, Bacillus virus CP51, Bacillus virus JL, Bacillus virus Shanette, Escherichia virus CVM10, Escherichia virus ep3, Erwinia virus Asesino, Erwinia virus EaH2, Pseudomonas virus EL, Halomonas virus HAP1, Vibrio virus VP882, Brevibacillus virus Jimmer, Brevibacillus virus Osiris, Pseudomonas virus Ab03, Pseudomonas virus KPP10, Pseudomonas virus PAKP3, Sinorhizobium virus M7, Sinorhizobium virus M12, Sinorhizobium virus N3, Erwinia virus Machina, Arthrobacter virus Brent, Arthrobacter virus Jawnski, Arthrobacter virus Martha, Arthrobacter virus Sonny, Edwardsiella virus MSW3, edwardsiella virus PEi21, Escherichia virus Mu, Shigella virus SfMu, Halobacterium virus phiH, Bacillus virus Grass, Bacillus virus NIT1, Bacillus virus SPG24, Aeromonas virus 43, Escherichia virus P1, Pseudomonas virus CAb1, Pseudomonas virus CAb02, Pseudomonas virus JG004, Pseudomonas virus PAKP1, Pseudomonas virus PAKP4, Pseudomonas virus PaP1, Burkholderia virus BcepF1, Pseudomonas virus 141, Pseudomonas virus Ab28, Pseudomonas virus DL60, Pseudomonas virus DL68, Pseudomonas virus F8, Pseudomonas virus JG024, Pseudomonas virus KPP12, Pseudomonas virus LBL3, Pseudomonas virus LMA2, Pseudomonas virus PB1, Pseudomonas virus SN, Pseudomonas virus PA7, Pseudomonas virus phiKZ, Rhizobium virus RHEph4, Ralstonia virus RSF1, Ralstonia virus RSL2, Ralstonia virus RSL1, Aeromonas virus 25, Aeromonas virus 31, Aeromonas virus Aes12, Aeromonas virus Aes508, Aeromonas virus AS4, Stenotrophomonas virus IME13, Staphylococcus virus IPLAC1C, Staphylococcus virus SEP1, Salmonella virus SPN3US, Bacillus virus 1, Geobacillus virus GBSV1, Yersinia virus R1RT, Yersinia virus TG1, Bacillus virus G, Bacillus virus PBS1, Microcystis virus Ma-LMMO1, Vibrio virus MAR, Vibrio virus VHML, Vibrio virus VP585, Bacillus virus BPS13, Bacillus virus Hakuna, Bacillus virus Megatron, Bacillus virus WPh, Acinetobacter virus AB3, Acinetobacter virus Abp1, Acinetobacter virus Fri1, Acinetobacter virus IME200, Acinetobacter virus PD6A3, Acinetobacter virus PDAB9, Acinetobacter virus phiAB1, Escherichia virus K30, Klebsiella virus K5, Klebsiella virus K11, Klebsiella virus Kp1, Klebsiella virus KP32, Klebsiella virus KpV289, Klebsiella virus F19, Klebsiella virus K244, Klebsiella virus Kp2, Klebsiella virus KP34, Klebsiella virus KpV41, Klebsiella virus KpV71, Klebsiella virus KpV475, Klebsiella virus SU503, Klebsiella virus SU552A, Pantoea virus Limelight, Pantoea virus Limezero, Pseudomonas virus LKA1, Pseudomonas virus phiKMV, Xanthomonas virus f20, Xanthomonas virus f30, Xylella virus Prado, Erwinia virus Era103, Escherichia virus K5, Escherichia virus K1-5, Escherichia virus K1E, Salmonella virus SP6, Escherichia virus T7, Kluyvera virus Kvpl, Pseudomonas virus ghl, Prochlorococcus virus PSSP7, Synechococcus virus P60, Synechococcus virus Syn5, Streptococcus virus Cp1, Streptococcus virus Cp7, Staphylococcus virus 44AHJD, Streptococcus virus C1, Bacillus virus B103, Bacillus virus GA1, Bacillus virus phi29, Kurthia virus 6, Actinomyces virus Av1, Mycoplasma virus P1, Escherichia virus 24B, Escherichia virus 933W, Escherichia virus Min27, Escherichia virus PA28, Escherichia virus Stx2 II, Shigella virus 7502Stx, Shigella virus POCJ13, Escherichia virus 191, Escherichia virus PA2, Escherichia virus TL2011, Shigella virus VASD, Burkholderia virus Bcep22, Burkholderia virus Bcepil02, Burkholderia virus Bcepmig1, Burkholderia virus DC1, Bordetella virus BPP1, Burkholderia virus BcepC6B, Cellulophaga virus Cba41, Cellulophaga virus Cba172, Dinoroseobacter virus DFL12, Erwinia virus Ea9-2, Erwinia virus Frozen, Escherichia virus phiV10, Salmonella virus Epsilon15, Salmonella virus SPN1S, Pseudomonas virus F116, Pseudomonas virus H66, Escherichia virus APEC5, Escherichia virus APEC7, Escherichia virus Bp4, Escherichia virus EC1UPM, Escherichia virus ECBP1, Escherichia virus G7C, Escherichia virus IME11, Shigella virus Sb1, Achromobacter virus Axp3, Achromobacter virus JWAlpha, Edwardsiella virus KF1, Pseudomonas virus KPP25, Pseudomonas virus R18, Pseudomonas virus Ab09, Pseudomonas virus LIT1, Pseudomonas virus PA26, Pseudomonas virus Ab22, Pseudomonas virus CHU, Pseudomonas virus LUZ24, Pseudomonas virus PAA2, Pseudomonas virus PaP3, Pseudomonas virus PaP4, Pseudomonas virus TL, Pseudomonas virus KPP21, Pseudomonas virus LUZ7, Escherichia virus N4, Salmonella virus 9NA, Salmonella virus SP069, Salmonella virus BTP1, Salmonella virus HK620, Salmonella virus P22, Salmonella virus ST64T, Shigella virus Sf6, Bacillus virus Page, Bacillus virus Palmer, Bacillus virus Pascal, Bacillus virus Pony, Bacillus virus Pookie, Escherichia virus 172-1, Escherichia virus ECB2, Escherichia virus NJO1, Escherichia virus phiEco32, Escherichia virus Septima11, Escherichia virus SU10, Brucella virus Pr, Brucella virus Tb, Escherichia virus Pollock, Salmonella virus FSL SP-058, Salmonella virus FSL SP-076, Helicobacter virus 1961P, Helicobacter virus KHP30, Helicobacter virus KHP40, Hamiltonella virus APSE1, Lactococcus virus KSY1, Phormidium virus WMP3, Phormidium virus WMP4, Pseudomonas virus 119X, Roseobacter virus SIO1, Vibrio virus VpV262, Vibrio virus VC8, Vibrio virus VP2, Vibrio virus VP5, Streptomyces virus Amela, Streptomyces virus phiCAM, Streptomyces virus Aaronocolus, Streptomyces virus Caliburn, Streptomyces virus Danzina, Streptomyces virus Hydra, Streptomyces virus Izzy, Streptomyces virus Lannister, Streptomyces virus Lika, Streptomyces virus Sujidade, Streptomyces virus Zemlya, Streptomyces virus ELB20, Streptomyces virus R4, Streptomyces virus phiHau3, Mycobacterium virus Acadian, Mycobacterium virus Baee, Mycobacterium virus Reprobate, Mycobacterium virus Adawi, Mycobacterium virus Bane1, Mycobacterium virus BrownCNA, Mycobacterium virus Chrisnmich, Mycobacterium virus Cooper, Mycobacterium virus JAMaL, Mycobacterium virus Nigel, Mycobacterium virus Stinger, Mycobacterium virus Vincenzo, Mycobacterium virus Zemanar, Mycobacterium virus Apizium, Mycobacterium virus Manad, Mycobacterium virus Oline, Mycobacterium virus Osmaximus, Mycobacterium virus Pg1, Mycobacterium virus Soto, Mycobacterium virus Suffolk, Mycobacterium virus Athena, Mycobacterium virus Bernardo, Mycobacterium virus Gadjet, Mycobacterium virus Pipefish, Mycobacterium virus Godines, Mycobacterium virus Rosebush, Mycobacterium virus Babsiella, Mycobacterium virus Brujita, Mycobacterium virus Che9c, Mycobacterium virus Sbash, Mycobacterium virus Hawkeye, Mycobacterium virus Plot, Salmonella virus AG11, Salmonella virus Entl, Salmonella virus f18SE, Salmonella virus Jersey, Salmonella virus L13, Salmonella virus LSPA1, Salmonella virus SE2, Salmonella virus SETP3, Salmonella virus SETP7, Salmonella virus SETP13, Salmonella virus SP101, Salmonella virus SS3e, Salmonella virus wks13, Escherichia virus K1G, Escherichia virus K1H, Escherichia virus Klind1, Escherichia virus Klind2, Salmonella virus SP31, Leuconostoc virus Lmd1, Leuconostoc virus LNO3, Leuconostoc virus LN04, Leuconostoc virus LN12, Leuconostoc virus LN6B, Leuconostoc virus P793, Leuconostoc virus 1A4, Leuconostoc virus Ln8, Leuconostoc virus Ln9, Leuconostoc virus LN25, Leuconostoc virus LN34, Leuconostoc virus LNTR3, Mycobacterium virus Bongo, Mycobacterium virus Rey, Mycobacterium virus Butters, Mycobacterium virus Michelle, Mycobacterium virus Charlie, Mycobacterium virus Pipsqueaks, Mycobacterium virus Xeno, Mycobacterium virus Panchino, Mycobacterium virus Phrann, Mycobacterium virus Redi, Mycobacterium virus Skinnyp, Gordonia virus BaxterFox, Gordonia virus Yeezy, Gordonia virus Kita, Gordonia virus Zirinka, Gorrdonia virus Nymphadora, Mycobacterium virus Bignuz, Mycobacterium virus Brusacoram, Mycobacterium virus Donovan, Mycobacterium virus Fishburne, Mycobacterium virus Jebeks, Mycobacterium virus Malithi, Mycobacterium virus Phayonce, Enterobacter virus F20, Klebsiella virus 1513, Klebsiella virus KLPN1, Klebsiella virus KP36, Klebsiella virus PKP126, Klebsiella virus Sushi, Escherichia virus AHP42, Escherichia virus AHS24, Escherichia virus AKS96, Escherichia virus C119, Escherichia virus E41c, Escherichia virus Eb49, Escherichia virus Jk06, Escherichia virus KP26, Escherichia virus Rogue 1, Escherichia virus ACGM12, Escherichia virus Rtp, Escherichia virus ADB2, Escherichia virus JMPW1, Escherichia virus JMPW2, Escherichia virus T1, Shigella virus PSf2, Shigella virus Shf11, Citrobacter virus Stevie, Escherichia virus TLS, Salmonella virus SP126, Cronobacter virus Esp2949-1, Pseudomonas virus Ab18, Pseudomonas virus Ab19, Pseudomonas virus PaMx11, Arthrobacter virus Amigo, Propionibacterium virus Anatole, Propionibacterium virus B3, Bacillus virus Andromeda, Bacillus virus Blastoid, Bacillus virus Curly, Bacillus virus Eoghan, Bacillus virus Finn, Bacillus virus Glittering, Bacillus virus Riggi, Bacillus virus Taylor, Gordonia virus Attis, Mycobacterium virus Barnyard, Mycobacterium virus Konstantine, Mycobacterium virus Predator, Mycobacterium virus Bernal13, Staphylococcus virus 13, Staphylococcus virus 77, Staphylococcus virus 108PVL, Mycobacterium virus Bron, Mycobacterium virus Faith1, Mycobacterium virus Joedirt, Mycobacterium virus Rumpelstiltskin, Lactococcus virus bIL67, Lactococcus virus c2, Lactobacillus virus c5, Lactobacillus virus Ld3, Lactobacillus virus Ldl7, Lactobacillus virus Ld25A, Lactobacillus virus LLKu, Lactobacillus virus phiLdb, Cellulophaga virus Cba121, Cellulophaga virus Cba171, Cellulophaga virus Cba181, Cellulophaga virus ST, Bacillus virus 250, Bacillus virus IEBH, Mycobacterium virus Ardmore, Mycobacterium virus Avani, Mycobacterium virus Boomer, Mycobacterium virus Che8, Mycobacterium virus Che9d, Mycobacterium virus Deadp, Mycobacterium virus Dlane, Mycobacterium virus Dorothy, Mycobacterium virus Dotproduct, Mycobacterium virus Drago, Mycobacterium virus Fruitloop, Mycobacterium virus Gumbie, Mycobacterium virus Ibhubesi, Mycobacterium virus Llij, Mycobacterium virus Mozy, Mycobacterium virus Mutaformal3, Mycobacterium virus Pacc40, Mycobacterium virus PMC, Mycobacterium virus Ramsey, Mycobacterium virus Rockyhorror, Mycobacterium virus SG4, Mycobacterium virus Shauna1, Mycobacterium virus Shilan, Mycobacterium virus Spartacus, Mycobacterium virus Taj, Mycobacterium virus Tweety, Mycobacterium virus Wee, Mycobacterium virus Yoshi, Salmonella virus Chi, Salmonella virus FSLSPO30, Salmonella virus FSLSP088, Salmonella virus iEPS5, Salmonella virus SPN19, Mycobacterium virus 244, Mycobacterium virus Bask21, Mycobacterium virus CJW1, Mycobacterium virus Eureka, Mycobacterium virus Kostya, Mycobacterium virus Porky, Mycobacterium virus Pumpkin, Mycobacterium virus Sirduracell, Mycobacterium virus Toto, Mycobacterium virus Corndog, Mycobacterium virus Firecracker, Rhodobacter virus RcCronus, Pseudomonas virus D3112, Pseudomonas virus DMS3, Pseudomonas virus FHA0480, Pseudomonas virus LPB1, Pseudomonas virus MP22, Pseudomonas virus MP29, Pseudomonas virus MP38, Pseudomonas virus PA1KOR, Pseudomonas virus D3, Pseudomonas virus PMG1, Arthrobacter virus Decurro, Gordonia virus Demosthenes, Gordonia virus Katyusha, Gordonia virus Kvothe, Propionibacterium virus B22, Propionibacterium virus Doucette, Propionibacterium virus E6, Propionibacterium virus G4, Burkholderia virus phi6442, Burkholderia virus phi1026b, Burkholderia virus phiE125, Edwardsiella virus eiAU, Mycobacterium virus Ff47, Mycobacterium virus Muddy, Mycobacterium virus Gaia, Mycobacterium virus Giles, Arthrobacter virus Captnmurica, Arthrobacter virus Gordon, Gordonia virus GordTnk2, Paenibacillus virus Harrison, Escherichia virus EK99P1, Escherichia virus HK578, Escherichia virus JL1, Escherichia virus SSL2009a, Escherichia virus YD2008s, Shigella virus EP23, Sodalis virus SO1, Escherichia virus HK022, Escherichia virus HK75, Escherichia virus HK97, Escherichia virus HK106, Escherichia virus HK446, Escherichia virus HK542, Escherichia virus HK544, Escherichia virus HK633, Escherichia virus mEp234, Escherichia virus mEp235, Escherichia virus mEpX1, Escherichia virus mEpX2, Escherichia virus mEp043, Escherichia virus mEp213, Escherichia virus mEp237, Escherichia virus mEp390, Escherichia virus mEp460, Escherichia virus mEp505, Escherichia virus mEp506, Brevibacillus virus Jenst, Achromobacter virus 83-24, Achromobacter virus JWX, Arthrobacter virus Kellezzio, Arthrobacter virus Kitkat, Arthrobacter virus Bennie, Arthrobacter virus DrRobert, Arthrobacter virus Glenn, Arthrobacter virus HunterDalle, Arthrobacter virus Joann, Arthrobacter virus Korra, Arthrobacter virus Preamble, Arthrobacter virus Pumancara, Arthrobacter virus Wayne, Mycobacterium virus Alma, Mycobacterium virus Arturo, Mycobacterium virus Astro, Mycobacterium virus Backyardigan, Mycobacterium virus BBPiebs31, Mycobacterium virus Benedict, Mycobacterium virus Bethlehem, Mycobacterium virus Billknuckles, Mycobacterium virus Bruns, Mycobacterium virus Bxb1, Mycobacterium virus Bxz2, Mycobacterium virus Che12, Mycobacterium virus Cuco, Mycobacterium virus D29, Mycobacterium virus Doom, Mycobacterium virus Ericb, Mycobacterium virus Euphoria, Mycobacterium virus George, Mycobacterium virus Gladiator, Mycobacterium virus Goose, Mycobacterium virus Hammer, Mycobacterium virus Heldan, Mycobacterium virus Jasper, Mycobacterium virus JC27, Mycobacterium virus Jeffabunny, Mycobacterium virus JHC117, Mycobacterium virus KBG, Mycobacterium virus Kssjeb, Mycobacterium virus Kugel, Mycobacterium virus L5, Mycobacterium virus Lesedi, Mycobacterium virus LHTSCC, Mycobacterium virus lockley, Mycobacterium virus Marcell, Mycobacterium virus Microwolf, Mycobacterium virus Mrgordo, Mycobacterium virus Museum, Mycobacterium virus Nepal, Mycobacterium virus Packman, Mycobacterium virus Peaches, Mycobacterium virus Perseus, Mycobacterium virus Pukovnik, Mycobacterium virus Rebeuca, Mycobacterium virus Redrock, Mycobacterium virus Ridgecb, Mycobacterium virus Rockstar, Mycobacterium virus Saintus, Mycobacterium virus Skipole, Mycobacterium virus Solon, Mycobacterium virus Switzer, Mycobacterium virus SWU1, Mycobacterium virus Ta17a, Mycobacterium virus Tiger, Mycobacterium virus Timshe1, Mycobacterium virus Trixie, Mycobacterium virus Turbido, Mycobacterium virus Twister, Mycobacterium virus U2, Mycobacterium virus Violet, Mycobacterium virus Wonder, Escherichia virus DE3, Escherichia virus HK629, Escherichia virus HK630, Escherichia virus lambda, Arthrobacter virus Laroye, Mycobacterium virus Halo, Mycobacterium virus Liefie, Mycobacterium virus Marvin, Mycobacterium virus Mosmoris, Arthrobacter virus Circum, Arthrobacter virus Mudcat, Escherichia virus N15, Escherichia virus 9g, Escherichia virus JenK1, Escherichia virus JenP1, Escherichia virus JenP2, Pseudomonas virus NP1, Pseudomonas virus PaMx25, Mycobacterium virus Baka, Mycobacterium virus Courthouse, Mycobacterium virus Littlee, Mycobacterium virus Omega, Mycobacterium virus Optimus, Mycobacterium virus Thibault, Polaribacter virus P12002L, Polaribacter virus P12002S, Nonlabens virus P12024L, Nonlabens virus P12024S, Thermus virus P23-45, Thermus virus P74-26, Listeria virus LP26, Listeria virus LP37, Listeria virus LP110, Listeria virus LP114, Listeria virus P70, Propionibacterium virus ATCC29399BC, Propionibacterium virus ATCC29399BT, Propionibacterium virus Attacne, Propionibacterium virus Keiki, Propionibacterium virus Kubed, Propionibacterium virus Lauchelly, Propionibacterium virus MrAK, Propionibacterium virus Ouroboros, Propionibacterium virus P91, Propionibacterium virus P105, Propionibacterium virus P144, Propionibacterium virus P1001, Propionibacterium virus P1.1, Propionibacterium virus P100A, Propionibacterium virus P100D, Propionibacterium virus P101A, Propionibacterium virus P104A, Propionibacterium virus PA6, Propionibacterium virus Pacnes201215, Propionibacterium virus PAD20, Propionibacterium virus PAS50, Propionibacterium virus PHL009M11, Propionibacterium virus PHL025M00, Propionibacterium virus PHL037M02, Propionibacterium virus PHL041M10, Propionibacterium virus PHL060L00, Propionibacterium virus PHL067M01, Propionibacterium virus PHL070N00, Propionibacterium virus PHL071N05, Propionibacterium virus PHL082M03, Propionibacterium virus PHL092M00, Propionibacterium virus PHL095N00, Propionibacterium virus PHL111M01, Propionibacterium virus PHL112N00, Propionibacterium virus PHL113M01, Propionibacterium virus PHL114L00, Propionibacterium virus PHL116M00, Propionibacterium virus PHL117M00, Propionibacterium virus PHL117M01, Propionibacterium virus PHL132N00, Propionibacterium virus PHL141N00, Propionibacterium virus PHL151M00, Propionibacterium virus PHL151N00, Propionibacterium virus PHL152M00, Propionibacterium virus PHL163M00, Propionibacterium virus PHL171M01, Propionibacterium virus PHL179M00, Propionibacterium virus PHL194M00, Propionibacterium virus PHL199M00, Propionibacterium virus PHL301M00, Propionibacterium virus PHL308M00, Propionibacterium virus Pirate, Propionibacterium virus Procrass1, Propionibacterium virus SKKY, Propionibacterium virus Solid, Propionibacterium virus Stormborn, Propionibacterium virus Wizzo, Pseudomonas virus PaMx28, Pseudomonas virus PaMx74, Mycobacterium virus Patience, Mycobacterium virus PBI1, Rhodococcus virus Pepy6, Rhodococcus virus Poco6, Propionibacterium virus PFR1, Streptomyces virus phiBT1, Streptomyces virus phiC31, Streptomyces virus TG1, Caulobacter virus Karma, Caulobacter virus Magneto, Caulobacter virus phiCbK, Caulobacter virus Rogue, Caulobacter virus Swift, Staphylococcus virus 11, Staphylococcus virus 29, Staphylococcus virus 37, Staphylococcus virus 53, Staphylococcus virus 55, Staphylococcus virus 69, Staphylococcus virus 71, Staphylococcus virus 80, Staphylococcus virus 85, Staphylococcus virus 88, Staphylococcus virus 92, Staphylococcus virus 96, Staphylococcus virus 187, Staphylococcus virus 52a, Staphylococcus virus 80alpha, Staphylococcus virus CNPH82, Staphylococcus virus EW, Staphylococcus virus IPLA5, Staphylococcus virus IPLA7, Staphylococcus virus IPLA88, Staphylococcus virus PH15, Staphylococcus virus phiETA, Staphylococcus virus phiETA2, Staphylococcus virus phiETA3, Staphylococcus virus phiMR11, Staphylococcus virus phiMR25, Staphylococcus virus phiNM1, Staphylococcus virus phiNM2, Staphylococcus virus phiNM4, Staphylococcus virus SAP26, Staphylococcus virus X2, Enterococcus virus FL1, Enterococcus virus FL2, Enterococcus virus FL3, Lactobacillus virus ATCC8014, Lactobacillus virus phiJL1, Pediococcus virus cIPi, Aeromonas virus pIS4A, Listeria virus LP302, Listeria virus PSA, Methanobacterium virus psiM1, Roseobacter virus RDJL1, Roseobacter virus RDJL2, Rhodococcus virus RER2, Enterococcus virus BC611, Enterococcus virus IMEEF1, Enterococcus virus SAP6, Enterococcus virus VD13, Streptococcus virus SPQS1, Mycobacterium virus Papyrus, Mycobacterium virus Send513, Burkholderia virus KL1, Pseudomonas virus 73, Pseudomonas virus Ab26, Pseudomonas virus Kakheti25, Escherichia virus Cajan, Escherichia virus Seurat, Staphylococcus virus SEP9, Staphylococcus virus Sextaec, Streptococcus virus 858, Streptococcus virus 2972, Streptococcus virus ALQ132, Streptococcus virus 01205, Streptococcus virus Sfi11, Streptococcus virus 7201, Streptococcus virus DT1, Streptococcus virus phiAbc2, Streptococcus virus Sfi19, Streptococcus virus Sfi21, Paenibacillus virus Diva, Paenibacillus virus Hb10c2, Paenibacillus virus Rani, Paenibacillus virus Shelly, Paenibacillus virus Sitara, Paenibacillus virus Willow, Lactococcus virus 712, Lactococcus virus ASCC191, Lactococcus virus ASCC273, Lactococcus virus ASCC281, Lactococcus virus ASCC465, Lactococcus virus ASCC532, Lactococcus virus Bibb29, Lactococcus virus bIL170, Lactococcus virus CB13, Lactococcus virus CB14, Lactococcus virus CB19, Lactococcus virus CB20, Lactococcus virus jj50, Lactococcus virus P2, Lactococcus virus P008, Lactococcus virus sk1, Lactococcus virus S14, Bacillus virus Slash, Bacillus virus Stahl, Bacillus virus Staley, Bacillus virus Stills, Gordonia virus Bachita, Gordonia virus ClubL, Gordonia virus OneUp, Gordonia virus Smoothie, Gordonia virus Soups, Bacillus virus SPbeta, Vibrio virus MAR10, Vibrio virus SSP002, Escherichia virus AKFV33, Escherichia virus BF23, Escherichia virus DT57C, Escherichia virus EPS7, Escherichia virus FFH1, Escherichia virus H8, Escherichia virus slur09, Escherichia virus T5, Salmonella virus 118970sal2, Salmonella virus Shivani, Salmonella virus SPC35, Salmonella virus Stitch, Arthrobacter virus Tank, Tsukamurella virus TIN2, Tsukamurella virus TIN3, Tsukamurella virus TIN4, Rhodobacter virus RcSpartan, Rhodobacter virus RcTitan, Mycobacterium virus Anaya, Mycobacterium virus Angelica, Mycobacterium virus Crimd, Mycobacterium virus Fionnbarth, Mycobacterium virus Jaws, Mycobacterium virus Larva, Mycobacterium virus Macncheese, Mycobacterium virus Pixie, Mycobacterium virus TM4, Bacillus virus BMBtp2, Bacillus virus TP21, Geobacillus virus Tp84, Staphylococcus virus 47, Staphylococcus virus 3a, Staphylococcus virus 42e, Staphylococcus virus IPLA35, Staphylococcus virus phi12, Staphylococcus virus phiSLT, Mycobacterium virus 32HC, Rhodococcus virus RGL3, Paenibacillus virus Vegas, Gordonia virus Vendetta, Bacillus virus Wbeta, Mycobacterium virus Wildcat, Gordonia virus Twister6, Gordonia virus Wizard, Gordonia virus Hotorobo, Gordonia virus Monty, Gordonia virus Woes, Xanthomonas virus CP1, Xanthomonas virus OP1, Xanthomonas virus phi17, Xanthomonas virus Xop411, Xanthomonas virus Xp10, Streptomyces virus TP1604, Streptomyces virus YDN12, Alphaproteobacteria virus phiJ1001, Pseudomonas virus LKO4, Pseudomonas virus M6, Pseudomonas virus MP1412, Pseudomonas virus PAE1, Pseudomonas virus Yua, Pseudoalteromonas virus PM2, Pseudomonas virus phi6, Pseudomonas virus phi8, Pseudomonas virus phi12, Pseudomonas virus phi13, Pseudomonas virus phi2954, Pseudomonas virus phiNN, Pseudomonas virus phiYY, Vibrio virus fs1, Vibrio virus VGJ, Ralstonia virus RS603, Ralstonia virus RSM1, Ralstonia virus RSM3, Escherichia virus M13, Escherichia virus 122, Salmonella virus IKe, Acholeplasma virus L51, Vibrio virus fs2, Vibrio virus VFJ, Escherichia virus If1, Propionibacterium virus B5, Pseudomonas virus Pf1, Pseudomonas virus Pf3, Ralstonia virus PE226, Ralstonia virus RSS1, Spiroplasma virus SVTS2, Stenotrophomonas virus PSH1, Stenotrophomonas virus SMA6, Stenotrophomonas virus SMA7, Stenotrophomonas virus SMA9, Vibrio virus CTXphi, Vibrio virus KSF1, Vibrio virus VCY, Vibrio virus Vf33, Vibrio virus VfO3K6, Xanthomonas virus Cf1c, Spiroplasma virus C74, Spiroplasma virus R8A2B, Spiroplasma virus SkV1CR23x, Escherichia virus F1, Escherichia virus Qbeta, Escherichia virus BZ13, Escherichia virus MS2, Escherichia virus alpha3, Escherichia virus ID21, Escherichia virus ID32, Escherichia virus ID62, Escherichia virus NC28, Escherichia virus NC29, Escherichia virus NC35, Escherichia virus phiK, Escherichia virus St1, Escherichia virus WA45, Escherichia virus G4, Escherichia virus ID52, Escherichia virus Talmos, Escherichia virus phiX174, Bdellovibrio virus MAC1, Bdellovibrio virus MH2K, Chlamydia virus Chp1, Chlamydia virus Chp2, Chlamydia virus CPAR39, Chlamydia virus CPG1, Spiroplasma virus SpV4, Acholeplasma virus L2, Pseudomonas virus PR4, Pseudomonas virus PRD1, Bacillus virus AP50, Bacillus virus Bam35, Bacillus virus GIL16, Bacillus virus Wipi, Escherichia virus phi80, Escherichia virus RB42, Escherichia virus T2, Escherichia virus T3, Escherichia virus T6, Escherichia virus VT2-Sa, Escherichia virus VT1-Sakai, Escherichia virus VT2-Sakai, Escherichia virus CP-933V, Escherichia virus P27, Escherichia virus Stx2phi-I, Escherichia virus Stx1phi, Escherichia virus Stx2phi-II, Escherichia virus CP-1639, , based on the Escherichia virus BP-4795, Escherichia virus 86, Escherichia virus Min27, Escherichia virus 2851, Escherichia virus 1717, Escherichia virus YYZ-2008, Escherichia virus EC026_P06, Escherichia virus ECO103_P15, Escherichia virus ECO103_P12, Escherichia virus ECO111_P16, Escherichia virus ECO111_P11, Escherichia virus VT2phi_272, Escherichia virus TL-2011c, Escherichia virus P13374, Escherichia virus Sp5.

In one embodiment, the bacterial delivery vehicles target E. coli and includes the capsid of a bacteriophage selected in the group consisting of BW73, B278, D6, D108, E, E1, E24, E41, F1-2, FI-4, FI-5, HI8A, Ff18B, i, MM, Mu, 025, PhI-5, Pk, PSP3, P1, PID, P2, P4, S1, Wφ, φK13, φ1, φ2, φ7, φ92, 7 A, 8φ, 9φ, 18, 28-1, 186, 299, HH-Escherichia (2), AB48, CM, C4, C16, Dd-VI, E4, E7, E28, FI1, FI3, H, H1, H3, H8, K3, M, N, ND-2, ND-3, ND4, ND-5, ND6, ND-7, Ox-I, Ox-2, Ox-3, Ox-4, Ox-5, Ox-6, PhI-I, RB42, RB43, RB49, RB69, S, Sal-I, Sal-2, Sal-3, Sal-4, Sal-5, Sa1-6, TC23, TC45, TuII*-6, TuIP-24, TulI*46, TuIP-60, T2, T4, T6, T35, α1, 1, IA, 3, 3A, 3T+, 5φ, 9266Q, CF0103, HK620, J, K, K1F, m59, no. A, no. E, no. 3, no. 9, N4, sd, T3, T7, WPK, W31, ΔH, φC3888, φK3, φK7, φK12, φV-1, Φ04-CF, Φ05, Φ06, Φ07, φ1, φ1.2, φ20, φ95, φ263, φ1O92, φ1, φ11, Ω8, 1, 3, 7, 8, 26, 27, 28-2, 29, 30, 31, 32, 38, 39, 42, 933W, NN-Escherichia (1), Esc-7-11, AC30, CVX-5, C1, DDUP, EC1, EC2, E21, E29, F1, F26S, F27S, Hi, HK022, HK97, HK139, HK253, HK256, K7, ND-I, PA-2, q, S2, Ti, ), T3C, T5, UC-I, w, β4, γ2, λ, ΦD326, φγ, Φ06, Φ7, Φ10, φ80, χ, 2, 4, 4A, 6, 8A, 102, 150, 168, 174, 3000, AC6, AC7, AC28, AC43, AC50, AC57, AC81, AC95, HK243, K10, ZG/3A, 5, 5A, 21EL, H19-J and 933H.

In embodiments wherein the genetic circuit is packaged in a bacterial delivery vehicle, said genetic circuit may comprise a nucleic acid sequence that signals for packaging and the donor cell may express bacteriophage scaffolding proteins. Said sequence that signals for packaging and said bacteriophage scaffolding proteins are chosen by the skilled person depending on the nature of the bacteriophage used as bacterial delivery vehicle.

In some embodiments, the bacterial donor cell and/or bacterial recipient cell disclosed herein may be used in the presence of prebiotics to enhance their growth or any other desired function of the bacterial donor cell and/or bacterial recipient cell. Prebiotics include, but are not limited to, amino acids, biotin, fructo-oligosaccharide, galacto-oligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carregenaan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides.

In a further embodiment, the nucleic acid sequence of interest contained in the genetic circuit and placed under the transcriptional control of a repressor binding sequence, encodes a protein conferring resistance to an antibiotic. As used herein, the term “antibiotic” refers to an antibiotic which is selected, for example, from the group consisting in penicillins such as penicillin G, penicillin K, penicillin N, penicillin 0, penicillin V, methicillin, benzylpenicillin, nafcillin, oxacillin, cloxacillin, dicloxacillin, ampicillin, amoxicillin, pivampicillin, hetacillin, bacampicillin, metampicillin, talampicillin, epicillin, carbenicillin, ticarcillin, temocillin, mezlocillin, and piperacillin; cephalosporins such as cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, loracarbef, cefbuperazone, cefminox, cefotetan, cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, latamoxef, cefclidine, cefepime, cefluprenam, cefoselis, cefozopran, cefpirome, cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; polymyxins such as polysporin, neosporin, polymyxin B, and polymyxin E, rifampicins such as rifampicin, rifapentine, and rifaximin; Fidaxomicin; quinolones such as cinoxacin, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, temafloxacin, tosufloxacin, clinafloxacin, gatifloxacin, gemifloxacin, moxifloxacin, sitafloxacin, trovafloxacin, prulifloxacin, delafloxacin, nemonoxacin, and zabofloxacin; sulfonamides such as sulfafurazole, sulfacetamide, sulfadiazine, sulfadimidine, sulfafurazole, sulfisomidine, sulfadoxine, sulfamethoxazole, sulfamoxole, sulfanitran, sulfadimethoxine, sulfametho-xypyridazine, sulfametoxydiazine, sulfadoxine, sulfametopyrazine, and terephtyl; macrolides such as azithromycin, clarithromycin, erythromycin, fidaxomicin, telithromycin, carbomycin A, josamycin, kitasamycin, midecamycin, oleandomycin, solithromycin, spiramycin, troleandomycin, tylosin, and roxithromycin; ketolides such as telithromycin, and cethromycin; lluoroketolides such as solithromycin; lincosamides such as lincomycin, clindamycin, and pirlimycin; tetracyclines such as demeclocycline, doxycycline, minocycline, oxytetracycline, and tetracycline; aminoglycosides such as amikacin, dibekacin, gentamicin, kanamycin, neomycin, netilmicin, sisomicin, tobramycin, paromomycin, and streptomycin; ansamycins such as geldanamycin, herbimycin, and rifaximin; carbacephems such as loracarbef; carbapenems such as ertapenem, doripenem, imipenem (or cilastatin), and meropenem; glycopeptides such as teicoplanin, vancomycin, telavancin, dalbavancin, and oritavancin; lincosamides such as clindamycin and lincomycin; lipopeptides such as daptomycin; monobactams such as aztreonam; nitrofurans such as furazolidone, and nitrofurantoin; oxazolidinones such as linezolid, posizolid, radezolid, and torezolid; teixobactin, clofazimine, dapsone, capreomycin, cycloserine, ethambutol, ethionamide, isoniazid, pyrazinamide, rifabutin, arsphenamine,chloramphenicol, fosfomycin, fusidic acid, metronidazole, mupirocin, platensimycin, quinupristin (or dalfopristin), thiamphenicol, tigecycline, tinidazole, trimethoprim, alatrofloxacin, fidaxomycin, nalidixice acide, rifampin, derivatives and combination thereof. In particular, the term “protein conferring resistance to an antibiotic” may refer to a protein conferring resistance to any of these antibiotics.

Provided are pharmaceutical or veterinary compositions comprising one or more of the bacterial delivery vehicles produced using the donor cells as disclosed herein, using the methods disclosed herein for producing bacterial delivery vehicles, and a pharmaceutically-acceptable carrier. The present disclosure also provides pharmaceutical or veterinary compositions comprising the recipient or target cells, such as for example a probiotic, where the genetic circuit has been transferred as disclosed herein, and a pharmaceutically-acceptable carrier. The present disclosure also provides pharmaceutical or veterinary compositions comprising the donor cells, such as for example a probiotic, as disclosed herein, i.e. comprising the genetic circuit and expressing the repressor protein, and a pharmaceutically-acceptable carrier. Generally, for pharmaceutical use, the bacterial delivery vehicles may be formulated as a pharmaceutical preparation or compositions comprising at least one bacterial delivery vehicles and at least one pharmaceutically acceptable carrier, diluent or excipient, and optionally one or more further pharmaceutically active compounds. Such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such administration forms may be solid, semi-solid or liquid, depending on the manner and route of administration. For example, formulations for oral administration may be provided with an enteric coating that will allow the synthetic bacterial delivery vehicles in the formulation to resist the gastric environment and pass into the intestines. More generally, synthetic bacterial delivery vehicle formulations for oral administration may be suitably formulated for delivery into any desired part of the gastrointestinal tract. In addition, suitable suppositories may be used for delivery into the gastrointestinal tract. Various pharmaceutically acceptable carriers, diluents and excipients useful in bacterial delivery vehicle compositions are known to the skilled person.

The pharmaceutical or veterinary composition according to the disclosure may further comprise a pharmaceutically acceptable vehicle. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. Suitable solid vehicles include, for example calcium phosphate, magnesium stearate, talc, sugars, lactose, dextrin, starch, gelatin, cellulose, polyvinylpyrrolidine, low melting waxes and ion exchange resins.

The pharmaceutical or veterinary composition may be prepared as a sterile solid composition that may be suspended at the time of administration using sterile water, saline, or other appropriate sterile injectable medium. The pharmaceutical or veterinary compositions of the disclosure may be administered orally in the form of a sterile solution or suspension containing other solutes or suspending agents (for example, enough saline or glucose to make the solution isotonic), bile salts, acacia, gelatin, sorbitan monoleate, polysorbate 8o (oleate esters of sorbitol and its anhydrides copolymerized with ethylene oxide) and the like. The particles according to the disclosure can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for enteral administration include sterile solutions, emulsions, and suspensions.

The bacterial delivery vehicles, produced according to the production method disclosed herein, may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and enteral administration include water (partially containing additives as above, e.g. cellulose derivatives, for example, sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for enteral administration. The liquid vehicle for pressurized compositions can be a halogenated hydrocarbon or other pharmaceutically acceptable propellant.

For transdermal administration, the pharmaceutical or veterinary composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compounds can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Also provided are methods for treating a disease or disorder caused by bacteria, such as a bacterial infection, using the compositions disclosed herein. In this aspect, the genetic circuit including the nucleic acid of interest is transferred in one or more bacteria causing the disease or disorder, i.e. the target cells. The methods include administering a pharmaceutical or veterinary composition disclosed herein, for example, a therapeutically effective amount of a pharmaceutical or veterinary composition disclosed herein, to a subject having a disease or disorder caused by bacteria, such as a bacterial infection, in need of treatment. Further provided is a pharmaceutical or veterinary composition as disclosed herein for use as a medicament, and in particular in the treatment of a disease or disorder caused by bacteria, such as in the treatment of a bacterial infection. Also provided is the use of a pharmaceutical or veterinary composition as disclosed herein for the manufacture of a medicament for treating a disease or disorder caused by bacteria, such as a bacterial infection.

In some embodiments, the subject is a mammal. In some particular embodiments, the subject is a human.

The disease or disorder caused by bacteria may be selected from the group consisting of abdominal cramps, acne vulgaris, acute epiglottitis, arthritis, bacteraemia, bloody diarrhea, botulism, Brucellosis, brain abscess, chancroid venereal disease, Chlamydia, Crohn's disease, conjunctivitis, cholecystitis, colorectal cancer, polyposis, dysbiosis, Lyme disease, diarrhea, diphtheria, duodenal ulcers, endocarditis, erysipelothricosis, enteric fever, fever, glomerulonephritis, gastroenteritis, gastric ulcers, Guillain-Barre syndrome tetanus, gonorrhoea, gingivitis, inflammatory bowel diseases, irritable bowel syndrome, leptospirosis, leprosy, listeriosis, tuberculosis, Lady Widermere syndrome, Legionaire's disease, meningitis, mucopurulent conjunctivitis, multi-drug resistant bacterial infections, multi-drug resistant bacterial carriage, myonecrosis-gas gangrene, Mycobacterium Avium complex, neonatal necrotizing enterocolitis, nocardiosis, nosocomial infection, otitis, periodontitis, phalyngitis, pneumonia, peritonitis, purpuric fever, Rocky Mountain spotted fever, shigellosis, syphilis, sinusitis, sigmoiditis, septicaemia, subcutaneous abscesses, tularaemia, tracheobronchitis, tonsillitis, typhoid fever, ulcerative colitis, urinary infection and whooping cough.

The bacterial infection may be selected from the group consisting of skin infections such as acne, intestinal infections such as esophagitis, gastritis, enteritis, colitis, sigmoiditis, rectitis, and peritonitis, urinary tract infections, vaginal infections, female upper genital tract infections such as salpingitis, endometritis, oophoritis, myometritis, parametritis and infection in the pelvic peritoneum, respiratory tract infections such as pneumonia, intra-amniotic infections, odontogenic infections, endodontic infections, fibrosis, meningitis, bloodstream infections, nosocomial infection such as catheter-related infections, hospital acquired pneumonia, post-partum infection, hospital acquired gastroenteritis, hospital acquired urinary tract infections, or a combination thereof. In an embodiment, the bacterial infection according to the disclosure is caused by a bacterium presenting an antibiotic resistance. In a particular embodiment, the infection is caused by a bacterium as listed above, a bacterium that can be used as donor or target cell.

Also provided is a method for treating a bacterial infection comprising administering to a subject having a bacterial infection in need of treatment the provided pharmaceutical or veterinary composition, in particular a therapeutically effective amount of the provided pharmaceutical or veterinary composition. A “therapeutically effective amount” is an amount which, when administered to a subject, is needed to treat the targeted disease or disorder, or to produce the desired effect, e.g. is needed to treat the disease or disorder caused by bacteria, in particular a bacterial infection.

A method for reducing the amount of virulent and/or antibiotic resistant bacteria in a bacterial population, in particular in a subject having a bacterial infection, is provided comprising contacting the bacterial population with a pharmaceutical or veterinary composition disclosed herein or with the bacterial delivery vehicles disclosed herein. Further provided is the use of a pharmaceutical or veterinary composition disclosed herein or a bacterial delivery vehicle disclosed herein for the manufacture of a medicament for reducing the amount of virulent and/or antibiotic resistant bacteria in a bacterial population, in particular in a subject having a bacterial infection.

The disclosure also concerns a pharmaceutical or veterinary composition for use in the treatment of a metabolic disorder including, for example, obesity, type 2 diabetes and nonalcoholic fatty liver disease. Indeed, emerging evidence indicates that these disorders are characterized by alterations in the intestinal microbiota composition and its metabolites [31]. The pharmaceutical or veterinary composition may thus be used to deliver in some intestinal bacteria a nucleic acid of interest which can alter the intestinal microbiota composition (e.g. by inducing death of some bacteria) or its metabolites (e.g. by inducing expression, overexpression or secretion of some molecules by said bacteria, for example molecules having a beneficial role on metabolic inflammation). The disclosure also concerns the use of a pharmaceutical or veterinary composition for the manufacture of a medicament for the treatment of a metabolic disorder including, for example, obesity, type 2 diabetes and nonalcoholic fatty liver disease. It also relates to a method for treating a metabolic disorder including, for example, obesity, type 2 diabetes and nonalcoholic fatty liver disease, comprising administering to a subject having a metabolic disorder in need of treatment the provided pharmaceutical or veterinary composition, in particular a therapeutically effective amount of the provided pharmaceutical or veterinary composition.

In a particular embodiment, the disclosure concerns a pharmaceutical or veterinary composition for use in the treatment of pathologies involving bacteria of the human microbiome, such as inflammatory and auto-immune diseases, cancers, infections or brain disorders. The disclosure also relates to a method for treating a pathology involving bacteria of the human microbiome comprising administering to a subject having said pathology and in need of treatment the provided pharmaceutical or veterinary composition, in particular a therapeutically effective amount of the provided pharmaceutical or veterinary composition, and relates to the use of a pharmaceutical or veterinary composition disclosed herein for the manufacture of a medicament for treating a pathology involving bacteria of the human microbiome. Indeed, some bacteria of the microbiome, without triggering any infection, can secrete molecules that will induce and/or enhance inflammatory or auto-immune diseases or cancer development. More specifically, the present disclosure relates also to modulating microbiome composition to improve the efficacy of immunotherapies based, for example, on CAR-T Φhimeric Antigen Receptor T) cells, TIL (Tumor Infiltrating Lymphocytes) and Tregs (Regulatory T cells) also known as suppressor T cells. Modulation of the microbiome composition to improve the efficacy of immunotherapies may also include the use of immune checkpoint inhibitors well known in the art such as, without limitation, PD-1 (programmed cell death protein 1) inhibitor, PD-L1 (programmed death ligand 1) inhibitor and CTLA-4 (cytotoxic T lymphocyte associated protein 4).

Some bacteria of the microbiome can also secrete molecules that will affect the brain.

Therefore, a further object of the disclosure is a method for controlling the microbiome of a subject, comprising administering an effective amount of the pharmaceutical composition as disclosed herein in said subject.

In a particular embodiment, the disclosure also relates to a method for personalized treatment for an individual in need of treatment for a bacterial infection comprising: i) obtaining a biological sample from the individual and determining a group of bacterial DNA sequences from the sample; ii) based on the determining of the sequences, identifying one or more pathogenic bacterial strains or species that were in the sample; and iii) administering to the individual a pharmaceutical composition according to the disclosure capable of recognizing each pathogenic bacterial strain or species identified in the sample and to deliver the packaged genetic circuit.

In an embodiment, the biological sample comprises pathological and non-pathological bacterial species, and subsequent to administering the pharmaceutical or veterinary composition according to the disclosure to the individual, the amount of pathogenic bacteria on or in the individual are reduced, but the amount of non-pathogenic bacteria is not reduced.

In another particular embodiment, the disclosure concerns a pharmaceutical or veterinary composition according to the disclosure for use in order to improve the effectiveness of drugs.

Indeed, some bacteria of the microbiome, without being pathogenic by themselves, are known to be able to metabolize drugs and to modify them in ineffective or harmful molecules.

In another particular embodiment, the disclosure concerns the in-situ bacterial production of any compound of interest, including therapeutic compound such as prophylactic and therapeutic vaccine for mammals. The compound of interest, encoded by the nucleic acid of interest comprised in the genetic circuit, can be produced inside the targeted bacteria, secreted from the targeted bacteria or expressed on the surface of the targeted bacteria. In a more particular embodiment, the compound of interest is an antigen expressed on the surface of the targeted bacteria for prophylactic and/or therapeutic vaccination.

The present disclosure also relates to a non-therapeutic use of the compositions disclosed herein. For instance, the non-therapeutic use can be a cosmetic use or a use for improving the well-being of a subject, in particular a subject who does not suffer from a disease. Accordingly, the present disclosure also relates to a cosmetic composition or a non-therapeutic composition comprising the compositions of the disclosure.

The present disclosure further provides kits for use in the transfer of a genetic circuit of interest from a donor cell to a recipient or target cell. In one embodiment, the kit comprises (i) a donor cell expressing a repressor protein; and (ii) a genetic circuit of interest. Said genetic circuit may be as defined above, in particular may comprise an expression cassette into which a nucleic acid of interest may be inserted in functional proximity to (is operably linked to) a repressor binding sequence recognized by the repressor protein. In another embodiment, or in addition, the donor cell of the kit may contain prophage sequences for generation of delivery vehicles, for example, packaging the genetic circuit of interest. The kit may further comprise a recipient or target cell wherein said recipient or target cell fails to express the repressor protein thereby permitting expression of the nucleic acid of interest following transfer into said cells.

In order to better understand the subject matter that is disclosed herein and to exemplify how it may be carried out in practice, embodiments will now be described, by way of non-limiting example, with reference to the accompanying drawings.

### Example 1

With specific reference to the examples, it is stressed that the particulars shown are by way of example and for purposes of illustrative discussion of embodiments disclosed herein.

The example below demonstrates the use of interspecific promoter-repressor pairs for use in a novel system for production of delivery vehicles. In this case, the repressor being expressed in trans in a production strain comes from a different bacterial species, ideally phylogenetically different. The use of interspecific repressors is a very advantageous tool to control the expression of a given protein or the transcription of a genetic circuit component only in the strain that contains the heterologous repressor in trans. Nevertheless, one needs to be careful when following this approach and not use repressors that may be found ubiquitously, such as the tryptophan synthesis repressor, since they have been shown to not be orthogonal when transferring them to other species [16]. But since bacteria inhabit environments with different characteristics, they have evolved specific repressors recognizing particular signals that may not be present at all in other organisms. Two recent publication shows that the use of interspecific repressors in E coli is possible [10] [17], representing a source of potential interspecific repressor-operator pairs, even if the inducers are not known. Finally, another solution involves the expression of an inactive Cas9 molecule (dCas9) and a gRNA/tracrRNA targeting the promoter, RBS or coding sequence of the toxic component. dCas9 does not have nuclease activity but it is able to bind and block transcription from the area targeted by the gRNA [18] [19].

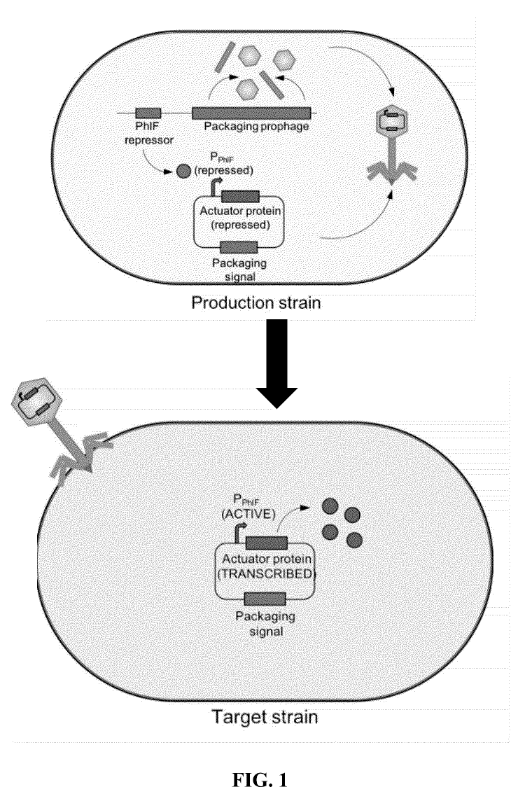

FIG. 1 depicts conditional transcriptional control with an interspecific repressor. On the left, the production strain, containing a packaging prophage and a PhlF (interspecific) repressor in trans. The payload carries the packaging signal and the desired sequence (actuator) under the control of a P_{phlF} promoter. Upon packaging of the phagemid particles, target strains can be transduced and the P_{phlF} promoter will be active since they lack the PhlF repressor (not present in E. coli). Note that the PhlF repressor can be replaced with a dCas9+gRNA targeting the promoter, RBS or sequence of the actuator.

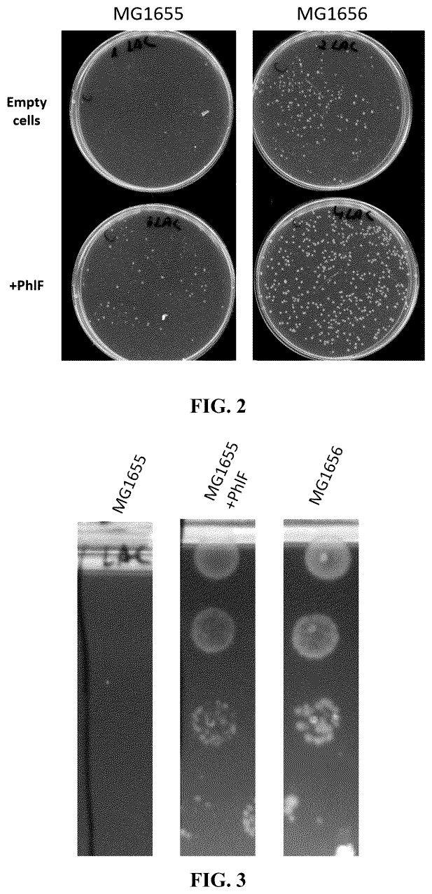

As a proof of concept, plasmids containing Cas9 under the control of the P_{phlF} promoter (SEQ ID NO: 3), an sgRNA targeting LacZ and a Lambda phage cos signal were constructed. Cas9 targeting LacZ has been previously used in a different setup [20]. Transformation of this plasmid into two strains of E. coli: MG1655 (wt strain) and MG1656 (contains a deletion in the lacZ) was first tried. As expected, the transformation in MG1655 yielded no colonies, since the Cas9 circuit targets its genome and it's toxic. In contrast, transformation into MG1656 gave colonies (FIG. 2).

Moreover, transformation into cells containing the PhlF repressor (SEQ ID NO:1, coding sequence: SEQ ID NO:2) expressed in trans in another plasmid gave colonies in both cases, since the repressor confers protection against Cas9 activity. The differences in size may be due to the fact that the constitutive expression of Cas9 has been shown to be toxic [21].

FIG. 2 depicts transformation of Cas9-containing circuits. Plasmids containing Cas9 under the control of a P_{phlF} promoter and a constitutive sgRNA guide targeting lacZ (SEQ ID NO:4) were transformed into MG1655 (left panels) or MG1656 (right panels). Empty cells (not carrying any other plasmid) are shown on the top; transformed cells containing an extra plasmid encoding the PhlF repressor (SEQ ID NO:5) are shown at the bottom.

To test if such a system could also be transduced, the PhlF repressor was integrated in the genome of the production strain, which also lacks the lacZ gene, and hence, is not targeted by Cas9. The production strain with this plasmid grew normally (data not shown). Phagemids were produced following a standard thermal induction protocol [23] and titrated on MG1655 and MG1656 (FIG. 3.). The transduction of Cas9-LacZ circuits into MG1655 gave no colonies, as in the case of transformation, while colonies were recovered in the case of MG1656.

FIG. 3 demonstrates transductions of Cas9-containing circuits. Phagemids containing Cas9 under the control of a P_{phF} promoter and a constitutive sgRNA guide targeting lacZ (SEQ ID NO:4) were transduced into MG1655 (left panel), MG1655 with the PhlF repressor encoded in a plasmid (center) (SEQ ID NO:5) or MG1656 (right panel).

Since the PhlF repressor does not naturally exist in E. coli, this system can be used to repress the expression of a toxic protein (in this case, Cas9) in the production strain while allowing for expression in another E. coli strain. In this specific case, sgRNA guides that do not target the production strain were used, but since Cas9 is repressed, the system would also allow for the production of phagemid particles encoding sgRNAs targeting its genome.

### Example 2

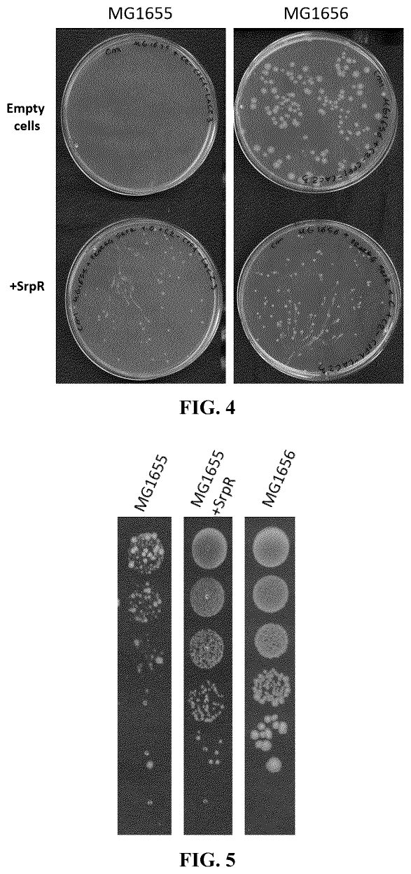

Plasmids containing the Cpf1 nuclease under the control of the P_{srpR} promoter (SEQ ID NO:8), a crRNA targeting LacZ and a Lambda phage cos signal φ455, SEQ ID NO:9) were constructed. Transformation of these plasmids into two strains of E. coli, MG1655 (wild-type strain) and MG1656 (contains a deletion in the lacZ gene), were first performed. As expected, the transformation in MG1655 yielded no colonies, since the Cpf1 circuit targets its genome and it's toxic. In contrast, transformation into MG1656 gave colonies (FIG. 4). Moreover, transformation into cells containing the SrpR repressor expressed in trans in another plasmid (pRARE4-SrpR-1.0, SEQ ID NO:10) gave colonies in both cases, since the repressor confers protection against Cpf1 activity (FIG. 4).

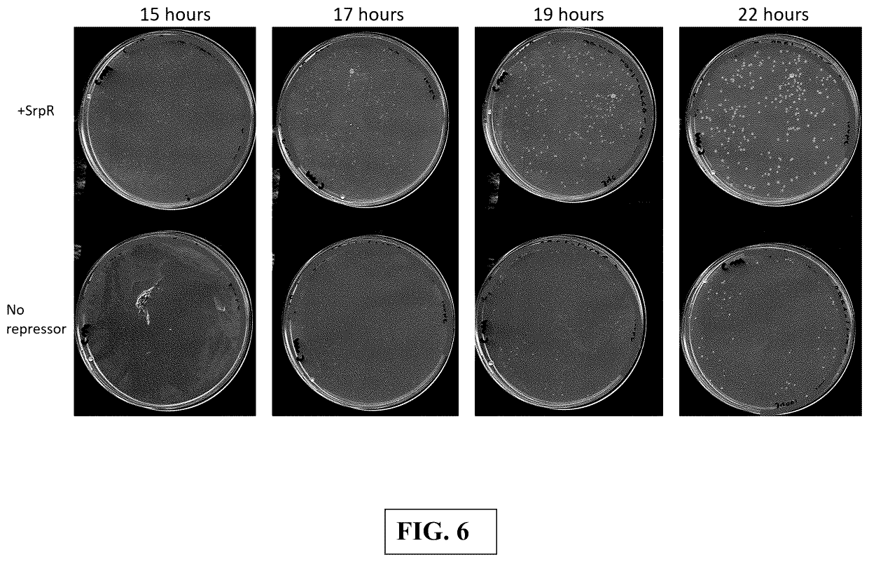

To test if such a system could also be transduced, the SrpR repressor (SEQ ID NO:6, coding sequence: SEQ ID NO:7) was integrated in the genome of the production strain, which also lacks the lacZ gene, and hence, is not targeted by Cpf1. Packaged phagemids were produced following a standard thermal induction protocol as indicated for the PhlF repressor data and titrated on MG1655 containing or not the SrpR repressor supplied in trans and MG1656 (FIG. 5). The transduction of Cpf1-LacZ circuits into MG1655 gave a reduction of almost 4 logs in the number of colonies recovered, reflecting the high toxicity of the circuit, while colonies were recovered in the case of MG1655 supplemented with the SrpR repressor to similar numbers as cells not containing the lacZ target (MG1656).

Finally, the addition of a repressor in the genome of the production strain that is able to repress in trans the payload may help reduce the burden of a circuit that would otherwise be constitutively expressed. Cells will be smaller, since their doubling time is reduced due to the plasmid burden, which could be detrimental for upscaling due to longer incubation times needed to reach a specific OD. Moreover, and perhaps more importantly, circuits that contain constitutively expressed components are unstable and prone to break fast, since the cell will find a way to remove the metabolic burden [24]-[30]. This is especially true if these expression levels are high, such as in the case of the Cpf1 circuit shown above: if the circuit breaks during a large-scale fermentation, this could lead to large economic losses.

FIG. 6 shows the addition of the SrpR repressor conferring a benefit to a production strain encoding a Cpf1-LacZ circuit in which the expression of Cpf1 is higher than in FIGS. 4 and 5 φ841, SEQ ID NO:11). Colony size after transformation of the P_{srpR}-Cpf1-LacZ circuit into a production strain containing the SrpR repressor or not was monitored.

Cells were transformed and incubated on chloramphenicol LB agar overnight at 30° C. and the size of the colonies tracked after 15h, 17h, 19h and 22h. As can be seen in FIG. 6, colonies are clearly seen at time 17h in the production strain containing the genomic SrpR repressor, but not in the one without. Even after 22 hours incubations, the colonies of the production strain without repressor are visibly smaller than those containing SrpR, which shows that the introduction of the repressor in trans reduces the metabolic burden in the production strain.

### List of References Cited

Any references cited in the specification are incorporated by reference herein in their entirety.
[1] L. Marschall, P. Sagmeister, and C. Herwig, “Tunable recombinant protein expression in E. coli: promoter systems and genetic constraints,” Appl. Microbiol. Biotechnol., vol. 101, no. 2, pp. 501-512, 2017.
[2] T. Brautaset, R. Lale, and S. Valla, “Positively regulated bacterial expression systems,” Microb. Biotechnol., vol. 2, no. 1, pp. 15-30, Jan. 2009.
[3] I. Mijakovic, D. Petranovic, and P. R. Jensen, “Tunable promoters in systems biology,” Curr. Opin. Biotechnol., vol. 16, no. 3, pp. 329-335, Jun. 2005.
[4] H. A. de Boer, L. J. Comstock, and M. Vasser, “The tac promoter: a functional hybrid derived from the trp and lac promoters,” Proc. Natd. Acad. Sci. U.S.A, vol. 80, no. 1, pp. 21-25, Jan. 1983.
[5] L. M. Guzman, D. Belin, M. J. Carson, and J. Beckwith, “Tight regulation, modulation, and high-level expression by vectors containing the arabinose PBAD promoter,” J. Bacteriol., vol. 177, no. 14, pp. 4121-4130, Jul. 1995.
[6] A. Skerra, “Use of the tetracycline promoter for the tightly regulated production of a murine antibody fragment in Escherichia coli,” Gene, vol. 151, no. 1, pp. 131-135, Dec. 1994.
[7] N. A. Valdez-Cruz, L. Caspeta, N. O. Pdréz, O. T. Ramírez, and M. A. Trujillo-Roldán, “Production of recombinant proteins in E. coli by the heat inducible expression system based on the phage lambda pL and/or pR promoters,” Microb. Cell Factories, vol. 9, p. 18, Mar. 2010.
[8] F. W. Studier and B. A. Moffatt, “Use of bacteriophage T7 RNA polymerase to direct selective high-level expression of cloned genes,” J. Mol. Biol., vol. 189, no. 1, pp. 113-130, May 1986.
[9] G. L. Rosano and E. A. Ceccarelli, “Recombinant protein expression in Escherichia coli: advances and challenges,” Front. Microbiol., vol. 5, Apr. 2014.
[10] B. C. Stanton, A. A. K. Nielsen, A. Tamsir, K. Clancy, T. Peterson, and C. A. Voigt,
“Genomic mining of prokaryotic repressors for orthogonal logic gates,” Nat. Chem. Biol., vol. 10, no. 2, pp. 99-105, Feb. 2014.
[11] F. Saïda, M. Uzan, B. Odaert, and F. Bontems, “Expression of highly toxic genes in E. coli: special strategies and genetic tools,” Curr. Protein Pept. Sci., vol. 7, no. 1, pp. 47-56, Feb. 2006.
[12] J. E. Cronan, “Cosmid-Based System for Transient Expression and Absolute Off-to-On Transcriptional Control of Escherichia coli Genes,” J. Bacteriol., vol. 185, no. 22, pp. 6522-6529, Nov. 2003.
[13] J. E. Cronan, “Improved Plasmid-Based System for Fully Regulated Off-To-On Gene Expression in Escherichia coli: Application to Production of Toxic Proteins,” Plasmid, vol. 69, no. 1, pp. 81-89, Jan. 2013.
[14] D. Bikard et al., “Exploiting CRISPR-Cas nucleases to produce sequence-specific antimicrobials,” Nat. Biotechnol., vol. 32, no. 11, pp. 1146-1150, Nov. 2014.
[15] J. Fernandez-Rodriguez, L. Yang, T. E. Gorochowski, D. B. Gordon, and C. A. Voigt, “Memory and Combinatorial Logic Based on DNA Inversions: Dynamics and Evolutionary Stability,” ACS Synth. Biol., vol. 4, no. 12, pp. 1361-1372, Dec. 2015.
[16] M. D. Manson and C. Yanofsky, “Tryptophan operon regulation in interspecific hybrids of enteric bacteria.,” J. Bacteriol., vol. 126, no. 2, pp. 679-689, May 1976.
[17] A. J. Meyer, T. H. Segall-Shapiro, E. Glassey, J. Zhang, and C. A. Voigt, “Escherichia coli ‘Marionette’ strains with 12 highly optimized small-molecule sensors,” Nat. Chem. Biol., vol. 15, no. 2, p. 196, Feb. 2019.
[18] D. Bikard, W. Jiang, P. Samai, A. Hochschild, F. Zhang, and L. A. Marraffini, “Programmable repression and activation of bacterial gene expression using an engineered CRISPR-Cas system,” Nucleic Acids Res., vol. 41, no. 15, pp. 7429-7437, Aug. 2013.
[19] L. S. Qi et al., “Repurposing CRISPR as an RNA-Guided Platform for Sequence-Specific Control of Gene Expression,” Cell, vol. 152, no. 5, pp. 1173-1183, Feb. 2013.
[20] L. Cui and D. Bikard, “Consequences of Cas9 cleavage in the chromosome of Escherichia coli,” Nucleic Acids Res., vol. 44, no. 9, pp. 4243-4251, May 2016.
[21] S. Cho, D. Choe, E. Lee, S. C. Kim, B. Palsson, and B.-K. Cho, “High-Level dCas9 Expression Induces Abnormal Cell Morphology in Escherichia coli,” ACS Synth. Biol., vol. 7, no. 4, pp. 1085-1094, Apr. 2018.
[22] J. Fernandez-Rodriguez, C.A. Voigt, “Post-translational control of genetic circuits using Potyvirus proteases.” Nucleic Acids Res., vol. 44, no. 13, pp. 6493-6502, Jul. 2016
[23] I. N. Wang, “Lysis Timing and Bacteriophage Fitness” Genetics, vol. 172, no 1, pp. 17-26, Jan 2006.
[24]S. C. Sleight, B. A. Bartley, J. A. Lieviant, and H. M. Sauro, “Designing and engineering evolutionary robust genetic circuits,” J. Biol. Eng., vol. 4, no. 1, p. 12, Nov. 2010, doi: 10.1186/1754-1611-4-12.
[25]M. B. Elowitz and S. Leibler, “A synthetic oscillatory network of transcriptional regulators,” Nature, vol. 403, no. 6767, pp. 335-338, Jan. 2000, doi: 10.1038/35002125.
[26]B. R. Glick, “Metabolic load and heterologous gene expression,” Biotechnol. Adv., vol. 13, no. 2, pp. 247-261, 1995.
[27]M. Scott, C. W. Gunderson, E. M. Mateescu, Z. Zhang, and T. Hwa, “Interdependence of Cell Growth and Gene Expression: Origins and Consequences,” Science, vol. 330, no. 6007, pp. 1099-1102, Nov. 2010, doi: 10.1126/science.1192588.
[28]S. C. Sleight and H. M. Sauro, “Visualization of Evolutionary Stability Dynamics and Competitive Fitness of Escherichia coli Engineered with Randomized Multigene Circuits,” ACS Synth. Biol., vol. 2, no. 9, pp. 519-528, Sep. 2013, doi: 10.1021/sb400055h.
[29]M. S. Bienick, K. W. Young, J. R. Klesmith, E. E. Detwiler, K. J. Tomek, and T. A. Whitehead,
“The Interrelationship between Promoter Strength, Gene Expression, and Growth Rate,” PLoS ONE, vol. 9, no. 10, p. e109105, Oct. 2014, doi: 10.1371/journal.pone.0109105.
[30]J. Fernandez-Rodriguez, L. Yang, T. E. Gorochowski, D. B. Gordon, and C. A. Voigt, “Memory and Combinatorial Logic Based on DNA Inversions: Dynamics and Evolutionary Stability,” ACS Synth. Biol., vol. 4, no. 12, pp. 1361-1372, Dec. 2015, doi: 10.1021/acssynbio.5b00170.
[31] Tilg et al., Nature Reviews Immunology, volume 20, pages 40-54, 2020

What is claimed:

1. A delivery vehicle obtained by a method of production of delivery vehicles, said method comprising:
(i) introducing a genetic circuit into a bacterial donor cell expressing a selected repressor protein, wherein said genetic circuit comprises a nucleic acid of interest under the transcriptional control of a repressor binding sequence recognized by said repressor protein, wherein said repressor protein is not encoded by said genetic circuit and said repressor protein and/or the repressor binding sequence originate from a different bacterial species than the bacterial donor cell; and
(ii) allowing a sufficient amount of time for replication of the genetic circuit and packaging of the genetic circuit to form the delivery vehicles comprising said genetic circuit.

2. The delivery vehicle of claim 1, wherein the method of production further comprises a step of purifying the delivery vehicle.

3. The delivery vehicle of claim 1, wherein said genetic circuit is a phagemid and said delivery vehicle is a packaged phagemid.

4. The delivery vehicle of claim 3, wherein the bacterial donor cell comprises prophage sequences encoding proteins required in trans for packaging of the phagemid to form packaged phagemids.

5. The delivery vehicle of claim 1, wherein said delivery vehicle is a bacteriophage.

6. The delivery vehicle of claim 1, wherein the selected repressor protein is selected from the group consisting of AmeR, AmrR, AmtR, ArpR, BarA, BarB, BM1P1, BM3R1, BpeR, ButR, Ca1R1, CampR, CasR, CprB, CymR, Cyp106, DhaR, Ef0113, EthR, FarA, HapR, HemR, HlyIIR, IcaR, IfeR, JadR2, KstR, LanK, LitR, LmrA, LuxT, McbR, MmfR, MtrR, NonG, OpaR, Orf2, orfL6, PaaR, PhlF, PqrA, PsbI, PsrA, Q9ZF45, QacR, RmrR, ScbR, SmcR, SmeT, SprR, TarA, TcmR, Th1R, TtgR, TtgW, TylP, TylQ, UrdK, VanT, VarR, YdeS, YDH1 and YixD.

7. The delivery vehicle of claim 6, wherein the selected repressor protein is selected from the group consisting of PhlF, SrpR, LitR, PsrA, AmeR, McbR, QacR, TarA, ButR, Orf2 and ScbR.

8. The delivery vehicle of claim 1, wherein the nucleic acid sequence of interest encodes a protein of interest and/or an RNA molecule of interest.

9. The delivery vehicle of claim 8, wherein said protein of interest is a toxic protein.

10. The delivery vehicle of claim 9, wherein said toxic protein is selected from the group consisting of holins, endolysins, restriction enzymes and toxins affecting the survival and/or the growth of a target cell.

11. The delivery vehicle of claim 8, wherein said protein of interest is a nuclease.

12. The delivery vehicle of claim 11, wherein the nuclease performs cleavage of a target bacterial cell genome or of a target bacterial cell plasmid.

13. The delivery vehicle of claim 8, wherein said protein of interest is a therapeutic protein.

14. The delivery vehicle of claim 8, wherein said RNA molecule of interest is selected from the group consisting of mRNA, crRNA, tRNA, iRNA, asRNA, ribozyme RNA, guide RNA and RNA aptamers.

15. The delivery vehicle of claim 1, wherein the nucleic acid sequence of interest encodes a CRISPR nuclease and the genetic circuit further comprises a nucleic acid sequence encoding a guide RNA.

16. The delivery vehicle of claim 15, wherein the nucleic acid sequence encoding a guide RNA is under the transcriptional control of a constitutive promoter.

17. The delivery vehicle of claim 1, wherein the nucleic acid of interest is a nucleic acid selected from the group consisting of a nucleic acid encoding an RNA, a toxin, an enzyme, a recombinase, a bacterial receptor, a membrane protein, a structural protein, a secreted protein, a protein conferring resistance to a drug, a toxic protein, a toxic factor, a virulence protein, a virulence factor, and any combination thereof.

18. The delivery vehicle of claim 17, wherein said enzyme is a nuclease or a kinase.

19. The delivery vehicle of claim 18, wherein the nuclease is selected from the group consisting of a Cas nuclease, a Cas9 nuclease, a TALEN, a ZFN and a meganuclease.

20. A pharmaceutical or veterinary composition comprising the delivery vehicle of claim 1 and a pharmaceutically acceptable excipient.
